(19) United States
(12) Patent Application Publication
(10) Pub. No.: US 2007/0078088 A1
(43) Pub. Date: Apr. 5, 2007

(54) Methods and compositions for treating cancer using 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 molecules

(75) Inventors: John Joseph Hunter, Somerville, MA (US); Kyle J. MacBeth, Boston, MA (US); Fong-Ying Tsai, Newton, MA (US); Andrea Lesoon, Concord, MA (US); Eric S. Lightcap, Natick, MA (US); Mark J. Williamson, Saugus, MA (US); Laura A. Rudolph-Owen, Medford, MA (US)

Correspondence Address: MILLENNIUM PHARMACEUTICALS, INC., 40 Landsdowne Street, CAMBRIDGE, MA, 02139, US

(73) Assignee: Millennium Pharmaceuticals, Inc.

(21) Appl. No.: 11/603,696

(22) Filed: Nov. 22, 2006

Related U.S. Application Data
(63) Continuation of application No. 10/354,358, filed on Jan. 30, 2003, now abandoned.
(60) Provisional application No. 60/353,600, filed on Jan. 31, 2002.
(60) Provisional application No. 60/364,517, filed on Mar. 15, 2002.
(60) Provisional application No. 60/371,075, filed on Apr. 9, 2002.
(60) Provisional application No. 60/371,507, filed on Apr. 10, 2002.
(60) Provisional application No. 60/372,984, filed on Apr. 16, 2002.
(60) Provisional application No. 60/374,194, filed on Apr. 19, 2002.
(60) Provisional application No. 60/382,995, filed on May 24, 2002.
(60) Provisional application No. 60/385,023, filed on May 31, 2002.
(60) Provisional application No. 60/388,853, filed on Jun. 14, 2002.
(60) Provisional application No. 60/389,395, filed on Jun. 17, 2002.
(60) Provisional application No. 60/391,324, filed on Jun. 25, 2002.
(60) Provisional application No. 60/395,944, filed on Jul. 15, 2002.
(60) Provisional application No. 60/397,726, filed on Jul. 22, 2002.
(60) Provisional application No. 60/403,046, filed on Aug. 13, 2002.
(60) Provisional application No. 60/405,155, filed on Aug. 22, 2002.
(60) Provisional application No. 60/406,361, filed on Aug. 27, 2002.
(60) Provisional application No. 60/421,195, filed on Oct. 25, 2002.
(60) Provisional application No. 60/425,456, filed on Nov. 12, 2002.
(60) Provisional application No. 60/427,626, filed on Nov. 19, 2002.
(60) Provisional application No. 60/432,122, filed on Dec. 10, 2002.

(51) Int. Cl. A61K 38/17; A61K 48/00; C12Q 1/68; G01N 33/574

(52) U.S. Cl. 514/12; 514/44; 435/6; 435/7.23

(57) ABSTRACT

The present invention relates to methods for the diagnosis and treatment of a cancer or cancer. Specifically, the present invention identifies the differential expression of 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 and 66428 genes in tissues relating to cancer, relative to their expression in normal, or non-cancer disease states, and/or in response to manipulations relevant to a cancer. The present invention describes methods for the diagnostic evaluation and prognosis of various cancers, and for the identification of subjects exhibiting a predisposition to such conditions. The invention also provides methods for identifying a compound capable of modulating a cancer or cancer. The present invention also provides methods for the identification and therapeutic use of compounds as treatments of cancer.

### RELATED APPLICATIONS

The present application is a continuation of U.S. patent application Ser. No. 10/354,358, filed Jan. 30, 2003 (pending), which claims the benefit of U.S. Provisional Application Ser. No. 60/353,600, filed on Jan. 31, 2002 (abandoned). U.S. patent application Ser. No. 10/354,358 also claims the benefit of U.S. Provisional Application Ser. No. 60/364,517, filed on Mar. 15, 2002 (abandoned); of U.S. Provisional Application Ser. No. 60/371,075, filed on Apr. 9, 2002 (abandoned); of U.S. Provisional Application Ser. No. 60/371,507, filed on Apr. 10, 2002 (abandoned); of U.S. Provisional Application Ser. No. 60/372,984, filed on Apr. 16, 2002 (abandoned); of U.S. Provisional Application Ser. No. 60/374,194, filed on Apr. 19, 2002 (abandoned); of U.S. Provisional Application Ser. No. 60/382,995, filed on May 24, 2002 (abandoned); of U.S. Provisional Application Ser. No. 60/385,023, filed on May 31, 2002 (abandoned); of U.S. Provisional Application Ser. No. 60/388,853, filed on Jun. 14, 2002 (abandoned); of U.S. Provisional Application Ser. No. 60/389,395, filed on Jun. 17, 2002 (abandoned); of U.S. Provisional Application Ser. No. 60/391,324, filed on Jun. 25, 2002 (abandoned); of U.S. Provisional Application Ser. No. 60/395,944, filed on Jul. 15, 2002 (abandoned); of U.S. Provisional Application Ser. No. 60/397,726, filed on Jul. 22, 2002 (abandoned); of U.S. Provisional Application Ser. No. 60/403,046, filed on Aug. 13, 2002 (abandoned); of U.S. Provisional Application Ser. No. 60/405,155, filed on Aug. 22, 2002 (abandoned); of U.S. Provisional Application Ser. No. 60/406,361, filed on Aug. 27, 2002 (abandoned); of U.S. Provisional Application Ser. No. 60/421,195, filed on Oct. 25, 2002 (abandoned); of U.S. Provisional Application Ser. No. 60/425,456, filed on Nov. 12, 2002 (abandoned); of U.S. Provisional Application Ser. No. 60/427,626, filed on Nov. 19, 2002 (abandoned); and of U.S. Provisional Application Ser. No. 60/432,122, filed on Dec. 10, 2002 (abandoned). The entire contents of these provisional patent applications are hereby incorporated by this reference.

### BACKGROUND OF THE INVENTION

Cancers can be viewed as a breakdown in the communication between tumor cells and their environment, including their normal neighboring cells. Growth-stimulatory and growth-inhibitory signals are routinely exchanged between cells within a tissue. Normally, cells do not divide in the absence of stimulatory signals or in the presence of inhibitory signals. In a cancerous or neoplastic state, a cell acquires the ability to “override” these signals and to proliferate under conditions in which a normal cell would not.

In general, tumor cells must acquire a number of distinct aberrant traits in order to proliferate in an abnormal manner. Reflecting this requirement is the fact that the genomes of certain well-studied tumors carry several different independently altered genes, including activated oncogenes and inactivated tumor suppressor genes. In addition to abnormal cell proliferation, cells must acquire several other traits for tumor progression to occur. For example, early on in tumor progression, cells must evade the host immune system. Further, as tumor mass increases, the tumor must acquire vasculature (e.g. through neo-angiogenesis) to supply nourishment and remove metabolic waste. Additionally, cells must acquire an ability to invade adjacent tissue. In many cases cells ultimately acquire the capacity to metastasize to distant sites.

Angiogenesis is a fundamental process by which new blood vessels are formed, as reviewed, for example, by Folkman and Shing, J. Biol. Chem. 267:10931-10934 (1992). Capillary blood vessels consist of endothelial cells and pericytes. These two cell types carry all of the genetic information to form tubes, branches and whole capillary networks. Specific angiogenic molecules and growth factors can initiate this process. Specific inhibitory molecules can stop it. These molecules with opposing function appear to be continuously acting in concert to maintain a stable microvasculature in which endothelial cell turnover is thousands of days. However, the same endothelial cells can undergo rapid proliferation, i.e. less than five days, during burst of angiogenesis, for example, during wound healing.

Key components of the angiogenic process are the degradation of the basement membrane, the migration and proliferation of capillary endothelial cell (EC) and the formation of three dimensional capillary tubes. The normal vascular turnover is rather low: the doubling time for capillary endothelium is from 50-20,000 days, but it is 2-13 days for tumor capillary endothelium. The current understanding of the sequence of events leading to angiogenesis is that a cytokine capable of stimulating endothelial cell proliferation, such as fibroblast growth factor (FGF), causes release of collagenase or plasminogen activator which, in turn, degrade the basement membrane of the parent venule to facilitate the migration of the endothelial cells. These capillary cells, having sprouted from the parent vessel, proliferate in response to growth factors and angiogenic agents in the surrounding environment to form lumen and eventually new blood vessels.

The development of a vascular blood supply is essential in reproduction, development and wound repair (Folkman, et al., Science 43:1490-1493 (1989)). Under these conditions, angiogenesis is highly regulated, so that it is turned on only as necessary, usually for brief periods of days, then completely inhibited. However, a number of serious diseases are also dominated by persistent unregulated angiogenesis and/or abnormal neovascularization including solid tumor growth and metastasis, psoriasis, endometriosis, Grave's disease, ischemic disease (e.g., atherosclerosis), and chronic inflammatory diseases (e.g., rheumatoid arthritis), and some types of eye disorders, (reviewed by Auerbach, et al., J. Microvasc. Res. 29:401-411 (1985); Folkman, Advances in Cancer Research, eds. Klein and Weinhouse, pp. 175-203 (Academic Press, New York, 1985); Patz, Am. J. Opthalmol. 94:715-743 (1982); and Folkman, et al., Science 221:719-725 (1983)). For example, there are a number of eye diseases, many of which lead to blindness, in which ocular neovascularization occurs in response to the diseased state. These ocular disorders include diabetic retinopathy, macular degeneration, neovascular glaucoma, inflammatory diseases and ocular tumors (e.g., retinoblastoma). There are a number of other eye diseases which are also associated with neovascularization, including retrolental fibroplasia, uveitis, eye diseases associated with choroidal neovascularization and eye diseases which are associated with iris neovascularization.

It is apparent that the complex process of tumor development and growth must involve multiple gene products. It is therefore important to define the role of specific genes involved in tumor development and growth and identify those genes and gene products that can serve as targets for the diagnosis, prevention and treatment of cancers.

In the realm of cancer therapy it often happens that a therapeutic agent that is initially effective for a given patient becomes, overtime, ineffective or less effective for that patient. The very same therapeutic agent may continue to be effective over a long period of time for a different patient. Further, a therapeutic agent that is effective, at least initially, for some patients can be completely ineffective or even harmful for other patients. Accordingly, it would be useful to identify genes and/or gene products that represent prognostic markers with respect to a given therapeutic agent or class of therapeutic agents. It then may be possible to determine which patients will benefit from particular therapeutic regimen and, importantly, determine when, if ever, the therapeutic regime begins to lose its effectiveness for a given patient. The ability to make such predictions would make it possible to discontinue a therapeutic regime that has lost its effectiveness well before its loss of effectiveness becomes apparent by conventional measures

### DESCRIPTION OF THE INVENTION

The present invention provides methods and compositions for the diagnosis and treatment of cancer, including but not limited to cancers of the lung, ovary, prostate, breast or colon, or conditions characterized by an increase or decrease in angiogenesis. The polypeptides and nucleic acids of the invention can also be used to treat, prevent, and/or diagnose cancers and neoplastic conditions in addition to the ones described above. As used herein, the terms “cancer”, “hyperproliferative” and “neoplastic” refer to cells having the capacity for autonomous growth, i.e., an abnormal state or condition characterized by rapidly proliferating cell growth. Hyperproliferative and neoplastic disease states may be categorized as pathologic, i.e., characterizing or constituting a disease state, or may be categorized as non-pathologic, i.e., a deviation from normal but not associated with a disease state. The term is meant to include all types of cancerous growths or oncogenic processes, metastatic tissues or malignantly transformed cells, tissues, or organs, irrespective of histopathologic type or stage of invasiveness. “Pathologic hyperproliferative” cells occur in disease states characterized by malignant tumor growth. Examples of non-pathologic hyperproliferative cells include proliferation of cells associated with wound repair.

Examples of cellular proliferative and/or differentiative disorders include cancer, e.g., carcinoma, sarcoma, or metastatic disorders. The molecules of the present invention can act as novel diagnostic targets and therapeutic agents for controlling breast cancer, ovarian cancer, colon cancer, lung cancer, prostatic cancer, squamous carcinoma of the cervix, as well as metastasis of such cancers and the like. A metastatic tumor can arise from a multitude of primary tumor types, including but not limited to those of breast, lung, liver, colon, ovarian origin, and colom-liver. A cellular proliferative disorder can be an endothelial cell disorder. As used herein, an “endothelial cell disorder” includes a disorder characterized by aberrant, unregulated, or unwanted endothelial cell activity, e.g., proliferation, migration, angiogenesis, or vascularization; or aberrant expression of cell surface adhesion molecules or genes associated with angiogenesis, e.g., TIE-2, FLT and FLK. Endothelial cell disorders include tumorigenesis, tumor metastasis, psoriasis, diabetic retinopathy, endometriosis, Grave's disease, ischemic disease (e.g., atherosclerosis), and chronic inflammatory diseases (e.g., rheumatoid arthritis).

Examples of cancers or neoplastic conditions, in addition to the ones described above, include, but are not limited to, a fibrosarcoma, myosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, gastric cancer, esophageal cancer, rectal cancer, pancreatic cancer, ovarian cancer, prostate cancer, uterine cancer, cancer of the head and neck, skin cancer, brain cancer, squamous cell carcinoma, sebaceous gland carcinoma, papillary carcinoma, papillary adenocarcinoma, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular cancer, small cell lung carcinoma, non-small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma, leukemia, lymphoma, or Kaposi sarcoma.

Examples of cellular proliferative and/or differentiative disorders of the breast include, but are not limited to, proliferative breast disease including, e.g., epithelial hyperplasia, sclerosing adenosis, and small duct papillomas; tumors, e.g., stromal tumors such as fibroadenoma, phyllodes tumor, and sarcomas, and epithelial tumors such as large duct papilloma; carcinoma of the breast including in situ (noninvasive) carcinoma that includes ductal carcinoma in situ (including Paget's disease) and lobular carcinoma in situ, and invasive (infiltrating) carcinoma including, but not limited to, invasive ductal carcinoma, invasive lobular carcinoma, medullary carcinoma, colloid (mucinous) carcinoma, tubular carcinoma, and invasive papillary carcinoma, and miscellaneous malignant neoplasms. Disorders in the male breast include, but are not limited to, gynecomastia and carcinoma.

Examples of cellular proliferative and/or differentiative disorders of the lung include, but are not limited to, bronchogenic carcinoma, including paraneoplastic syndromes, bronchioloalveolar carcinoma, neuroendocrine tumors, such as bronchial carcinoid, miscellaneous tumors, and metastatic tumors; pathologies of the pleura, including inflammatory pleural effusions, noninflammatory pleural effusions, pneumothorax, and pleural tumors, including solitary fibrous tumors (pleural fibroma) and malignant mesothelioma. Preferred examples of lung tumors that can be treated include small cell carcinoma and poorly differentiated small cell carcinoma of the lung.

Examples of cellular proliferative and/or differentiative disorders of the colon include, but are not limited to, non-neoplastic polyps, adenomas, familial syndromes, colorectal carcinogenesis, colorectal carcinoma, and carcinoid tumors. Preferred examples of colon tumors include moderately differentiated tumors.

Examples of cellular proliferative and/or differentiative disorders of the ovary include, but are not limited to, ovarian tumors such as, tumors of coelomic epithelium, serous tumors, mucinous tumors, endometeriod tumors, clear cell adenocarcinoma, cystadenofibroma, brenner tumor, surface epithelial tumors; germ cell tumors such as mature (benign) teratomas, monodermal teratomas, immature malignant teratomas, dysgerminoma, endodermal sinus tumor, choriocarcinoma; sex cord-stomal tumors such as, granulosa-theca cell tumors, thecoma-fibromas, androblastomas, hill cell tumors, and gonadoblastoma; and metastatic tumors such as Krukenberg tumors.

Examples of prostatic cancerous disorders include adenocarcinoma or carcinoma, of the prostate and/or testicular tumors.

Examples of conditions characterized by an increase or decrease in angiogenesis include but are not limited to solid tumor growth and metastasis, psoriasis, endometriosis, Grave's disease, ischemic disease (e.g., atherosclerosis), and chronic inflammatory diseases (e.g., rheumatoid arthritis), and some types of eye disorders

“Treatment”, as used herein, is defined as the application or administration of a therapeutic agent to a patient, or application or administration of a therapeutic agent to an isolated tissue or cell line from a patient, who has a disease or disorder, a symptom of disease or disorder or a predisposition toward a disease or disorder, with the purpose of curing, healing, alleviating, relieving, altering, remedying, ameliorating, improving or affecting the disease or disorder, at least one symptom of disease or disorder or the predisposition toward a disease or disorder. A therapeutic agent includes, but is not limited to, small molecules, peptides, antibodies, ribozymes, gene therapy vectors and antisense oligonucleotides. Representative molecules are described herein.

The present invention is based, at least in part, on the discovery that nucleic acid and protein molecules, (described infra), are differentially expressed in disease states relative to their expression in normal, or non-disease states. The modulators of the molecules of the present invention, identified according to the methods of the invention, can be used to modulate (e.g., inhibit, treat, or prevent) or diagnose a disease, including, but not limited to, a cancer including but not limited to cancers of the lung, ovary, prostate, breast, colon or other disease state characterized by modulation of angiogenesis.

The modulators of the molecules of the present invention can include but are not limited to small organic molecules, peptides, ribozymes, nucleic acid antisense molecules, gene therapy vectors or antibodies.

“Differential expression”, as used herein, includes both quantitative as well as qualitative differences in the temporal and/or tissue expression pattern of a gene. Thus, a differentially expressed gene may have its expression activated or inactivated in normal versus disease conditions. The degree to which expression differs in normal versus disease or control versus experimental states need only be large enough to be visualized via standard characterization techniques, e.g., quantitative PCR, Northern analysis, subtractive hybridization. The expression pattern of a differentially expressed gene may be used as part of a prognostic or diagnostic of a disease, e.g., a cancer including but not limited to cancers of the lung, ovary, prostate, breast, colon or other disease state characterized by modulation of angiogenesis evaluation, or may be used in methods for identifying compounds useful for the treatment of a disease, e.g., a cancer including but not limited to cancers of the lung, ovary, prostate, breast or colon. In addition, a differentially expressed gene involved in a disease may represent a target gene such that modulation of the level of target gene expression or of target gene product activity will act to cure, heal, alleviate, relieve, alter, remedy, ameliorate, improve or affect a disease condition, e.g., a cancer including but not limited to cancers of the lung, ovary, prostate, breast, colon or other disease state characterized by modulation of angiogenesis. Compounds that modulate target gene expression or activity of the target gene product can be used in the treatment of a disease. Although the genes described herein may be differentially expressed with respect to a disease, and/or their products may interact with gene products important to a disease, the genes may also be involved in mechanisms important to additional disease cell processes.

Molecules of the Present Invention

The molecules of the present invention can be characterized as, or have structural features in common with, molecules of the following functional classes, including but not limited to:

Transferases:

MTAP/PNP family of phosphorylases

2-oxo acid dehydrogenases acyltransferase

adenylate-kinase

1-acyl-sn-glycerol-3-phosphate acyltransferase

AIR synthase and relatives

class II aldolase domain

Aminotransferases

AMP-binding enzymes

arginine N-methyltransferase

Arginosuccinate synthase

NAD:arginine ADP-ribosyltransferase

Asparagine synthase

Asp and Glu kinases

ATP:guanido phosphotransferases

ATP synthase

bile acid CoA:amino acid N-acyltransferase

Biopterin-dependent aromatic amino acid hydroxylase

biotin-requiring enzymes

Beta-ketoacyl synthase

biotin-protein ligase

Carbohydrate phosphorylases

carnitate acyltransferase

CDP-alcohol phosphatidyltransferase

choline transferases

CoA ligases

Coenzyme A transferase

Cys/Met metabolism PLP-dependent enzyme

diacylglycerol kinase

Delta-aminolevulinic acid dehydratase

Dihydrodipicolinate synthetase family

Enol-ase

FGGY carbohydrate kinase family

Formyl transferase

fucosyltransferases

Galactose-1-phosphate uridyl transferase

galactosyl-transferases

Phosphoribosylglycinamide synthetase (GARS)

Type I glutamine amidotransferases

Type II glutamine amidotransferases

gamma-glutamyltransferase

GHMP kinases

Glutamine synthetase

glycosyl tferases group 2

type 4 glycosyl transferases

Glycosyl transferases group 1

guanylate cyclases

Hexokinase

Hydroxymethylglutaryl-coenzyme A synthase

Lyase

vitamin-B12 dependent methionine synthase

mRNA capping enzyme

arylamine N-acetyltransferase

nucleoside diphosphate kinase

glucosaminyl N-deacetylase/N-sulphotransferase

Myristoyl-CoA:protein N-myristoyltransferase

NNMT/PNMT/TEMT methyltransferase family

Nucleotidyl transferase

6-O-methylguanine DNA methyltransferase

Orotidine phosphate decarboxylases

O-methyltransferase

OTCase/ATCase

phenylalanine and histidine ammonia-lyases

poly(ADP-ribose) polymerase

Phosphatidate cytidylyltransferase

phosphoenolpyruvate carboxykinase

pfkB family carbohydrate kinase

Phosphofructokinase

Phosphoglycerate kinases

phosphoinositol-3-kinases

phosphatidylinositol-4-phosphate 5-kinase

eukaryotic protein kinases

polyprenyl synthetases

protein prenyltransferases

Purine/pyrimidine phosphoribosyl transferases

Phosphoribosyl pyrophosphate synthetase

6-pyruvoyl tetrahydropterin synthase

Pyridoxal-dependent decarboxylase

Pyridoxal-dependent decarboxylase conserved domain

pyridoxine kinases

pyruvate-kinase

Rhodanese

Ribosomal RNA adenine dimethylases

S-adenosylmethionine synthetase

SAICAR synthetase

Serine hydroxymethyltransferase

sialyltransferases

sterol O-acyltransferases

SpoU rRNA Methylase family

Squalene and phytoene synthases

serine/threonine dehydratases

sulfotransferases

Transaldolase

Trehalose-6-phosphate synthase domain

Tetrapyrrole (Corrin/Porphyrin) Methylases.

thymidine kinase

thiopurine methyltransferase

Thiamine Pyrophosphate requiring enzymes

Transglutaminase family

Transketolase

thymidylate synthase

ubiE/COQ5 methyltransferase family

UDP-glycosyltransferase

vitamin-K dependent gamma carboxylase

Oxidoreductases:

D-isomer specific 2-hydroxyacid dehydrogenase

3-beta hydroxysteroid dehydrogenase/isomerase

3-hydroxyacyl-CoA dehydrogenase

Acyl-CoA dehydrogenases

Zinc-containing alcohol dehydrogenases

adrenodoxin oxidoreductase

AhpC/TSA antioxidant enzyme family

aldehyde dehydrogenases

aldo/keto reductases

billiverdin reductase family

C-4 methyl sterol oxidase

C-5 cytosine-specific DNA methylase

cyclooxygenases

copper amine oxidases

FAD/NAD-binding Cytochrome reductase

D-amino acid oxidases

Molybdopterin binding domain in dehydrogenase

fatty acid desaturases

Dihydrofolate reductase

E1 dehydrogenases

Glutamate/Leucine/Phenylalanine/aline dehydrogena

FAD-dependent glycerol-3-phosphate dehydrogenase

FMN-dependent dehydrogenase

Flavin-binding monooxygenase-like

Glucose-6-phosphate dehydrogenase

glutathione peroxidases

GMC oxidoreductases

IMP dehydrogenase/GMP reductase

Isocitrate and isopropylmalate dehydrogenases

lactate/malate dehydrogenase

lipoxygenase

NAD dependent epimerase/dehydratase family

NAD-dependent glycerol-3-phosphate dehydrogenase

NADH dehydrogenases

NADH-ubiquinone/plastoquinone oxidoreductase chain

Nitroreductase family

NO Synthase

Oxidoreductase FAD/NAD-binding domain

Delta 1-pyrroline-5-carboxylate reductase

6-phosphogluconate dehydrogenases

Alanine dehydrogenase/pyridine nucleotide transhyd

Oxidoreductase molybdopterin binding domain

ribonuclease reductases

steroid 5-alpha reductases

short-chain dehydrogenase/reductases

Succinate dehydrogenase cytochrome b subunit

Tetrahydrofolate dehydrogenase/cyclohydrolase

UDP-glucose/GDP-mannose dehydrogenases

Hydrolases:

alpha/beta hydrolases

acid ceramidase

acylphosphatase

acyl-transferase

adenosine deaminase

S-adenosyl-L-homocysteine hydrolase

AdoMet decarboxylase

amidases

arginases

Asparaginase

aspartyl proteases

astacin/m12a metalloproteases

Prenyl protease 2

Eukaryotic carbonic anhydrases

carboxylesterase

Clp family of ATP-dependent proteases

2′,3′ cyclic nucleotide 3′ phosphodiesterase

cytidine deaminases

disintegrin

dUTPase

esterases

Fructose-1-6-bisphosphatase

Alpha-L-fucosidase

metalloprotease family

Glycosyl hydrolase family 1

hyaluronidases

GTP cyclohydrolase I

haloacid dehalogenase-like hydrolases

hemoglobinase

heparanase

histone deacetylases

insulinase

lipoprotein lipase et al

lysophospholipases

peptidase family m17

metalloprotease family M41

leishmanolysin family of metalloproteases

M24 proteases

matrix metalloproteases

mutT/8-OXO-dGTPase

neprilysin family of proteases

nucleotide pyrophosphatase (alkaline phosphodieste

procollagen N-proteinase

3′5′-cyclic nucleotide phosphodiesterase

ArgE/DapE/Acy1/Cpg2 family

Phosphorylase family

phospholipase A2

phospholipase C

phospholipase D

Porphobilinogen deaminase

pyrophosphatases

prolyl oligopeptidases

pyrimidine-nucleoside phosphorylases

GTPase-activators for Ras-like GTPases

renaldipeptidase

ADAM family of metalloprotease

serine carboxypeptidases

subtilase family of proteases

Sulfatase

Thioesterase domain

Thiolase

trehalase

trypsin-like serine proteases

Uracil-DNA glycosylase

Zinc carboxypeptidases

Zinc proteases

Isomerases:

enoyl-CoA hydratase/isomerase

sterol isomerase

Glucosamine-6-phosphate isomerase

Glyoxalase

Mannose-6-phosphate isomerase (fam1)

methylacyl-CoA racemase

Macrophage migration inhibitory factor (MIF)

Phosphoglucose isomerase

phosphoglucomutase/phosphomannomutase

Phosphoglycerate mutase family

Triosephosphate isomerase

tRNA pseudouridine synthase

Other Enzymes and Receptors:

phorbol ester/DAG binding domain

phospholipid scramblase

Nuclear hormone receptors

G-protein coupled receptors

Serine/threonine kinases

Tyrosine kinases

Dual specificity kinases

Gene ID 140

The human 140 sequence (SEQ ID NO:1), known also as N-formylpeptide receptor (fMLP-R26), is approximately 1281 nucleotides long including untranslated regions. The coding sequence, located at about nucleic acids 62 to 1114 of SEQ ID NO:1, encodes a 350 amino acid protein (SEQ ID NO:2).

As assessed by TaqMan expression analysis and in situ hybridization, 140 mRNA was found to be upregulated in a number of human tumors over normal control tissues, including but not limited to tumors of the breast, lung and colon. In addition, 140 mRNA was found to be upregulated in 3/3 glioblastomas as compared to normal brain samples. Due to its expression in a number of human tumors, modulators of 140 activity would be useful in treating human cancers, including but not limited to cancers of the breast, lung, colon and brain.

Further TaqMan studies show that 140 mRNA was expressed in a number of angiogenic tissues, including but not limited to mouse yolk sac, fetal liver, and angiogenic and tumorigenic islets cells from the mouse. Due to the increased expression of 140 mRNA in angiogenic tissues, modulators of 140 activity would be useful in treating conditions characterized by aberrant angiogenesis.

Gene ID 1470

The human 1470 sequence (SEQ ID NO:3), known also as protein kinase receptor (HEK2), is approximately 3805 nucleotides long including untranslated regions. The coding sequence, located at about nucleic acids 28 to 3024 of SEQ ID NO:3, encodes a 998 amino acid protein (SEQ ID NO:4).

As assessed by TaqMan analysis, 1470 mRNA was found to be upregulated in ovarian, prostate, colon (15x) and lung tumors when compared to normal human tissues. Further analysis showed increased expression in lung and colon tumors (4/4), liver metastases (1/1) and 16/16 colon to liver metastases. Phase II ACA (define): Upregulated expression in all stages of CRC (define) including 6/6 liver metastases. Gene 1470 or (EPB3/HEK2) belongs to the family of receptor tyrosine kinases (RTKs). In general, RTKs govern signal transduction signals that control cellular growth and differentiation. In particular, Ephrin type A+B receptor tyrosine kinases are known to be involved in axonal pathfinding, neurodevelopmental processes and the regulation of cellular proliferation. Defects in RTKs play a role in tumorigenesis.

The in situ hybridization (ISH) experiment for 1470 indicates that primary colon tumors and metastatic colon cancer to the liver are the primary tissues that express this gene. Normal colonic epithelium does not express 1470 mRNA. There is some expression in breast tumors and slight expression seen in one normal breast sample. Lung tumors do not appreciably express this gene.

1470 is expressed and upregulated in colon tumors. 1470 mRNA was upregulated in 100% of liver metastases and >75% primary colon tumors studied as assessed by TaqMan analysis. 1470 is involved in proliferation pathways, as it is upregulated in adenomas from the APC^{min} mouse model. The involvement of 1470 in intracellular pathways indicates that 1470 would be useful as a target for a cancer therapeutic. Due to its increased expression in colon tumors indicate that modulators of 1470 activity would be useful therapeutics in treating cancer, including but not limited to cancers of the colon.

Gene ID 1686

The human 1686 sequence (SEQ ID NO:5), known also as human receptor-type tyrosine kinase (rse), is approximately 3949 nucleotides long including untranslated regions. The coding sequence, located at about nucleic acids 225 to 2897 of SEQ ID NO:5, encodes an 890 amino acid protein (SEQ ID NO:6).

As assessed by TaqMan analysis, 1686 mRNA was found to be upregulated in fetal adrenal gland, fetal kidney and fetal heart. In addition, it was found to be upregulated in angiogenic tissues, including but not limited to Wilm's tumors, glioblastomas and neuroblastomas. Further studies indicated that 1686 mRNA was upregulated in breast and lung tumors, proliferating endothelial cells and endothelial cells undergoing tube formation. Further, 1686 mRNA was upregulated in HUVEC and HMVEC treated with complete medium. Due to its increased expression in a number of human tumors and angiogenic tissue, modulators of 1686 activity would be useful as therapeutics in treating cancer and those conditions characterized by aberrant angiogenesis.

Gene ID 2089

The human 2089 sequence (SEQ ID NO:7), known also as a eukaryotic protein kinase, is approximately 2470 nucleotides long including untranslated regions. The coding sequence, located at about nucleic acids 171 to 2126 of SEQ ID NO:7, encodes a 651 amino acid protein (SEQ ID NO:8).

Expression of 2089 mRNA was determined in the following human breast cell lines:

MCF10A.m25, MCF10AT.cl1, MCF10AT.cl3, MCF10AT1, MCF10AT3B and MCF10CA1.cl1. MCF10A cells are normal human breast epithelial cells, as they are near diploid, do not grow in soft agar and are nontumorigenic in nude mice. MCF10AT.cl1 and MCF10AT.cl3 cells are clones of the MCF10AT line, a T24 Hras-transfected MCF10A cell line. The MCF10AT cell line is tumorigenic as a pool, but these clones are not tumorigenic. MCF10AT1, MCF10AT3B and MCF10CA.cl1cells are cell lines derived from the in vitro expansion of cells taken from a MCF10AT(X)-derived xenograft tumors in nude mice. In comparison to MCF10AT1 cells, MCF10AT3B cells progress to invasive carcinomas more frequently. The MCF10CA1a.cl1 cell line is the most rapidly tumorigenic. 2089 mRNA was found to be upregulated in the tumorigenic cell lines as compared to the non-tumorigenic cell lines.

TaqMan analysis showed moderate expression in many tumors, with high expression seen in lung and colon tumors. There was no expression of 2089 mRNA in normal human tissues. Further TaqMan analysis on 2089 mRNA showed that 2089 mRNA was upregulated in pools of tumors of the breast, ovary, colon and lung vs. respective normals, and high expression in colon to liver metastases, with expression seen in all metastatic tissues tested.

2089 is a serine/kinase with increased expression in breast epithelial cells that have been transformed with activated H-ras. Additionally, expression was higher in primary solid tumors and metastases than in respective normal tissues. The increased expression of 2089 mRNA in tumors indicates that modulators of 2089 would be useful as therapeutics in treating cancer. 2089 would be useful as a target to identify cancer therapeutics.

Gene ID 2427

The human 2427 sequence (SEQ ID NO:9), known also as P2Y6 receptor, is approximately 1571 nucleotides long including untranslated regions. The coding sequence, located at about nucleic acids 277 to 1263 of SEQ ID NO:9, encodes a 328 amino acid protein (SEQ ID NO:10).

As assessed by TaqMan analysis 2427 mRNA was upregulated in tumor samples as compared to normal tissues. Due to the increased expression level of 2427 mRNA in tumor cells, modulators of 2427 activity have utility as cancer therapeutics.

Gene ID 3702

The human 3702 sequence (SEQ ID NO:11), known also as a tyrosine kinase is approximately 1467 nucleotides long including untranslated regions. The coding sequence, located at about nucleic acids 1 to 1467 of SEQ ID NO:I 1, encodes a 488 amino acid protein (SEQ ID NO:12).

As assessed by TaqMan analysis, 3702 mRNA was upregulated in lung, colon, breast and ovarian tumors as compared to the normal human tissue. In situ hybridization confirmed expression in the epithelial component of the tumor samples of 3702 mRNA.

3702 is a src-family kinase. Members of this family are known oncogenes that play a role in cellular proliferation. Due to the increased expression of 3702, it may be acting to promote or maintain unregulated proliferation in tumor cells. Therefore, modulators of 3702 activity are useful as cancer therapeutics and 3702 polypeptides are useful to screen for small molecule modulators of 3702 activity.

Gene ID 5891

The human 5891 sequence (SEQ ID NO:13), known also as a human prolyl oligopeptidase, is approximately 2562 nucleotides long including untranslated regions. The coding sequence, located at about nucleic acids 1 to 2133 of SEQ ID NO:13, encodes a 710 amino acid protein (SEQ ID NO:14).

As assessed by TaqMan analysis, 5891 mRNA was upregulated in 4/4 colon tumors and 2/2 liver metastases as compared to the normal human tissue. Further TaqMan studies showed that 5891 mRNA was upregulated in 15/16 liver metastases over normal liver. In addition, 5891 mRNA was down regulated in HCT116 k-ras deficient cell lines.

Prolyl oligopeptidases are highly specific endopeptidases. These enzymes function to degrade a variety of proline-containing peptides such as Substance P. Activities of these enzymes cleave peptide bonds at the carboxy side of proline residues. Proline is the only cyclic amino acid, and it has a rigid structure. These factors prevent the classical serine endopeptidases to cleave peptide bonds containing proline. Prolyl oligopeptidases are the only proline specific endopeptidases currently known in mammals.

Prolyl oligopeptidases have been shown to have activity in a wide distribution of tissues. Literature has shown that prolyl oligopeptidase activity was significantly higher in human prostate, lung and sigmoid tumors when-compared to healthy normal tissues. It has also been indicated that PE plays a role in cell cycle control.

5891 is upregulated in colon carcinomas and colon to liver metastases. TaqMan analyses show that 5891 has elevated expression levels in >90% of all stages of colon carcinomas tested. Due to the high level of expression of 5891 in colon tumors, modulators of 5891 activity are useful as cancer therapeutics, including but not limited to colon cancer. 5891 polypeptides are useful in discovering small molecule modulators of 5891 activity.

Gene ID 6428

The human 6428 sequence (SEQ ID NO:15), known also as alpha enolase, is approximately 1755 nucleotides long including untranslated regions. The coding sequence, located at about nucleic acids 95 to 1399 of SEQ ID NO:15, encodes a 434 amino acid protein (SEQ ID NO:16).

As assessed by TaqMan analysis, 6428 mRNA was upregulated in colon, lung, ovarian and breast tumors. In addition, further TaqMan studies indicated that 6428 mRNA was increased in angiogenic tumors including but not limited to Wilms' tumors and glioblastomas, and fetal liver, heart and kidney. 6428 mRNA was upregulated at day 10.5 yolk sac and in proliferating endothelial cells and endothelial cells undergoing tube formation. Also, 6428 was upregulated in cells that were cultured under conditions of hypoxia.

Due to its expression pattern in a number of human tumor tissues and angiogenic tissues, modulators of 6428 activity would be useful as cancer therapeutics as therapeutics in conditions characterized by aberrant angiogenesis. 6428 polypeptides would be useful in screening therapeutic compounds.

Gene ID 7181

The human 7181 sequence (SEQ ID NO:17), known also as HU-K4, is approximately 2131 nucleotides long including untranslated regions. The coding sequence, located at about nucleic acids 488 to 1801 of SEQ ID NO:17, encodes a 437 amino acid protein (SEQ ID NO:18).

Gene ID 7660

The human 7660 sequence (SEQ ID NO:19), known as chemokine receptor D6, is approximately 1181 nucleotides long including untranslated regions. The coding sequence, located at about nucleic acids 13 to 1167 of SEQ ID NO:19, encodes a 384 amino acid protein (SEQ ID NO:20).

As assessed by TaqMan analyses, 7660 mRNA was upregulated in a number of ovarian tumors as compared to normal ovarian tissue. Therefor, due to this increased expression in ovarian tumors, modulators of 7660 activity are useful as cancer therapeutics. 7660 polypeptides are useful to screen for therapeutic modulators.

Gene ID 25641

The human 25641 sequence (SEQ ID NO:21), known also as histone deacetylase (HDAC9), is approximately 3036 nucleotides long including untranslated regions. The coding sequence, located at about nucleic acids 1 to 3036 of SEQ ID NO:21, encodes a 1011 amino acid protein (SEQ ID NO:22).

As assessed by TaqMan analyses, there was a significant increase in 25641 mRNA expression in breast and ovarian tumors vs. respective normals, with high levels of expression seen in brain, spinal cord, and cardiovascular tissues.

25641 expression was increased in primary solid tumors and metastases in comparison to respective normal tissues. General histone deacetylase (HDAC) inhibitors are known to inhibit cell cycle and inhibit tumor progression in xenograft models. Therefore, due to the expression of 25641 in a number of human tumors, modulators of 25641 activity are useful as cancer therapeutic. 25641 polypeptides are useful in identifying modulators of 25641 activity.

Gene ID 69583

The human 69583 sequence (SEQ ID NO:23), is approximately 5549 nucleotides long including untranslated regions. The coding sequence, located at about nucleic acids 1 to 3111 of SEQ ID NO:23, encodes a 1036 amino acid protein (SEQ ID NO:24).

As assessed by TaqMan analysis, 69583 mRNA was upregulated in colon, lung and ovarian tumors. Expression was also seen in the kidney and pancreas. In situ hybridization confirmed the TaqMan results indicating that there was high level of 69583 mRNA in primary breast and ovarian tumors.

69583 mRNA was upregulated in a number of tumors. Therefore, modulators of 69583 activity would be useful as cancer therapeutics. 69583 polypeptides would be useful to identify modulators of 69583 activity.

Gene ID 49863

The human 49863 sequence (SEQ ID NO:25), known also sphingosine kinase 1 (SPK-1), is approximately 1799 nucleotides long including untranslated regions. The coding sequence, located at about nucleic acids 359 to 1513 of SEQ ID NO:25, encodes a 384 amino acid protein (SEQ ID NO:26).

As assessed by TaqMan analysis, 49863 mRNA was upregulated in tumors of the lung, colon and ovary. In addition 49863 mRNA was upregulated in angiogenic tumors and fetal tissue. It was highly expressed in human microvascular endothelial cells. As assessed by ISH expression analysis, 49863 mRNA was upregulated in tumor epithelium and stroma of breast, lung, colon and angiogenic tissues. Due to its expression pattern and functional role, modulators of 49863 activity would be useful in treating cancer and conditions characterized by aberrant angiogenesis. 49863 polypeptides of the present invention would be useful in screening for modulators of 49863 activity.

Gene ID 8897

The human 8897 sequence (SEQ ID NO:27), known also as Acyl-Co A Dehydrogenase, is approximately 2682 nucleotides long including untranslated regions. The coding sequence, located at about nucleic acids 16 to 1314 of SEQ ID NO:27, encodes a 432 amino acid protein (SEQ ID NO:28).

As assessed by TaqMan expression analysis, 8897 mRNA was found to be upregulated in a number of human tumors over normal control tissues, including but not limited to tumors of the lung and colon. Due to its expression in a number of human tumors, modulators of 8897 activity would be useful in treating human cancers, including but not limited to cancers of the lung and colon. 8897 polypeptides of the present invention would be useful in screening for modulators of 8897 activity.

Gene ID 1682

The human 1682 sequence (SEQ ID NO:29), known also as Tau-Tubulin Kinase (TTK), is approximately 3866 nucleotides long including untranslated regions. The coding sequence, located at about nucleic acids 978 to 3551 of SEQ ID NO:29, encodes a 857 amino acid protein (SEQ ID NO:30).

As assessed by TaqMan analysis, 1682 mRNA was found to be upregulated expression in blood cells, lung, ovary, colon and breast tumors when compared to normal human tissues. Additional TaqMan analysis in rodent and human breast cancer panels showed that 1682 was expressed in a subset of breast, ovary, lung and colon tumors. 1682 or (TTK) expression is associated with cell proliferation belonging to the family of serine/threonine family of protein kinases. Members of this gene family are involved in cell cycle progression.

As assessed by TaqMan analysis, 1682 mRNA expression was upregulated in transformed and tumorigenic breast epithelial cells as compared to normal breast epithelial cells.

In situ hybridization experiments for 1682 indicate that primary breast and ovarian carcinomas express this gene preferentially. Modest expression is seen in a subset of primary colon and metastatic colon carcinomas to the liver.

Published data showed that 1682 expression was associated with rapidly proliferating tissues, including malignant tumors. These data indicate that 1682 is a target for discovering novel therapeutics useful in treating cancer. The increased expression of 1682 mRNA in breast epithelial cells indicate that modulators of 1682 activity would be useful therapeutics in treating cancer, including but not limited to cancers of the breast. 1682 polypeptides of the present invention would be useful in screening for modulators of 1682 activity.

Gene ID 17667

The human 17667 sequence (SEQ ID NO:31), known also as Carboxypeptidase D (CPD), is approximately 5801 nucleotides long including untranslated regions. The coding sequence, located at about nucleic acids 36 to 4169 of SEQ ID NO:31, encodes a 1377 amino acid protein (SEQ ID NO:32).

As assessed by TaqMan analysis, 17667 mRNA was ubiquitously expressed with significant upregulation in tumors of the breast, ovarian, lung, and colon. Additional TaqMan analysis in human breast cancer panels and oncology cell panels showed that 17667 was expressed in high to extremely high levels when compared to that of normal tissues. In situ hybridization experiments (ISH) demonstrated that 17667 is expressed in a subset of lung, breast, and colon tumors and in an ovarian metastasis.

17667 (CPD) is a type 1 transmembrane protein with three tandem carboxypeptidase homology domains (Rehlil, T. F., et al, 1997; Ishikawa, T., et al, 1998). Two of these domains possess all of the catalytically important residues and a third domain is predicted to be catalytically inactive (Rehlil, T. F., et al, 1997; Ishikawa, T., et al, 1998). Human CPD is a homolog of duck gp180 and is a cellular receptor for avian hepatitis B virus entry (Breiner, K. M., et al, 1998). CPD is predominantly localized in the trans Golgi network (TGN) and cycles between the Golgi and the cell surface (Varlamov, O., et al, 1999). CPD is also found in immature, but not mature, secretory granules (Varlamov, O., et al, 1999), however, an isoform of CPD has been identified that is nuclear localized and inducible by prolactin in MCF-7 cells (Too, C. K., et al, 2001). CPD is believed to be involved in processing of proteins that transit the secretory pathway.

The high levels of 17667 mRNA seen in areas involved in tumors of the breast, ovary, lung and colon suggests that 17667 is involved in the processing of peptide hormones affecting tumor growth and/or survival. Therefore, inhibition of this 17667 would inhibit tumor progression. Modulators of 17667 activity would be useful therapeutics in treating cancer. 17667 polypeptides of the present invention would be useful in screening for modulators of 17667 activity.

Gene ID 9235

The human 9235 sequence (SEQ ID NO:33), known also as Glutamine Fructose-6-Phosphate Amidotransferase 1 (GFAT1), is approximately 3082 nucleotides long including untranslated regions. The coding sequence, located at about nucleic acids 123 to 2168 of SEQ ID NO:33, encodes a 681 amino acid protein (SEQ ID NO:34).

As assessed by TaqMan analysis, 9235 mRNA showed a higher expression in breast, lung, and colon tumor relative to normal colon tissues. Further experiments in an expanded colon and metastases panel showed generally higher expression in adenomas, stage B and C relative to normal colon tissues and higher expression in liver metastases relative to normal liver tissues.

The restricted pattern of 9235 mRNA expression in areas involved in tumors of the breast, lung and colon suggests that 9235 may be the rate-limiting enzyme in the hexosamine pathway determining the levels of UDP-GlcNAc in the cell. Published data has shown that UDP-GlcNAc is a key modifier of proteins influencing tumor metastatic potential and oncogene transcriptional activity. Therefore, inhibition of 9235 would result in loss of metastatic potential and effect a decrease in signaling through proliferation, growth factor, and antiapoptosis pathways. Therefore, modulators of 9235 activity would be useful therapeutics in treating cancer. 9235 polypeptides of the present invention would be useful in screening for modulators of 9235 activity.

Gene ID 3703

The human 3703 sequence (SEQ ID NO:35), known also as a serine/threonine protein kinase, is approximately 3224 nucleotides long including untranslated regions. The coding sequence, located at about nucleic acids 65 to 1309 of SEQ ID NO:35, encodes a 414 amino acid protein (SEQ ID NO:36).

As assessed by TaqMan analysis 3703 mRNA was upregulated in colon and lung tumor samples as compared to normal tissues. Due to the increased expression level of 3703 mRNA in tumor cells, modulators of 3703 activity have utility as cancer therapeutics. 3703 polypeptides of the present invention would be useful in screening for modulators of 3703 activity.

Gene ID 14171

The human 14171 sequence (SEQ ID NO:37), known also as Protein Kinase C Delta-Interactin Kinase (DIK), is approximately 3860 nucleotides long including untranslated regions. The coding sequence, located at about nucleic acids 17 to 2371 of SEQ ID NO:37, encodes a 784 amino acid protein (SEQ ID NO:38).

As assessed by TaqMan expression analysis, 14171 mRNA expression was increased in ovary, colon and lung tumor samples as compared to their respective normal tissues. Kidney, pancreas and liver also expressed 14171 mRNA. Ovarian and breast cancer cell model panels showed that 14171 mRNA was expressed in many serous ovarian tumors and clear cell tumors as wells as primary breast tumors. A lung cancer cell model panel showed 14171 mRNA expression is reduced in H125 cells induced to express p53 vs. noninduced cells or induced vector control cells. The ISH experiment for 14171 indicates that lung and ovarian primary carcinomas show the greatest expression of this gene. The gene is expressed slightly less in colon primaries and colon metastasis to the liver. Normal epithelial cells are negative.

14171 is a p53-repressed kinase that is expressed in tumors and interacts with PKCdelta. 14171 is involved in the negative regulations of proapoptotic functions of PKCdelta, thereby enhancing survival of tumor cells. Published data suggests that inhibition of this enzyme promotes apoptosis in tumor cells. Due to 14171 mRNA expression in a number of human tumors and its functional role, modulators of 14171 activity would be useful in treating human cancers, including but not limited to cancers of the ovary, colon, lung and breast. 14171 polypeptides of the present invention would be useful in screening for modulators of 14171 activity.

Gene ID 10359

The human 10359 sequence (SEQ ID NO:39), known also as Glutamine Phophoribosylpyrophosphate Amidotransferase (GPAT) is approximately 2153 nucleotides long including untranslated regions. The coding sequence, located at about nucleic acids 122 to 1675 of SEQ ID NO:39, encodes a 517 amino acid protein (SEQ ID NO:40).

As assessed by TaqMan analysis, 10359 mRNA was expressed in breast, ovary, prostate, colon, lung, brain and uterus tumor tissues as compared to their normal counterparts. Breast cancer cell model panels showed that 10359 mRNA expression was higher in estrogen receptor (ER) positive when compared to ER-negative breast tumor cell lines.

10359 is a type-2 glutamine amidotransferase that catalyzes the first step in de novo purine biosynthesis. The activity of purine metabolizing enzymes, including amidophosphoribosyltransferase, has previously been shown to be increased in colon carcinomas versus normal mucosa. Rapidly proliferating cancer cells have increased nucleotide requirements. Increased expression of 10359 mRNA supports the increased nucleotide requirements of tumor cells. Therefore, inhibition of 10359 can inhibit tumor progression. Due to its increased expression in breast, ovary, prostate, colon, lung, brain and uterine tumors, and its functional role, modulators of 10359 activity would be useful in treating cancer. 10359 polypeptides of the present invention would be useful in screening for modulators of 10359 activity.

Gene ID 1660

The human 1660 sequence (SEQ ID NO:41), known also as a human receptor tyrosine kinase (RON), is approximately 4541 nucleotides long including untranslated regions. The coding sequence, located at about nucleic acids 29 to 4231 of SEQ ID NO:41, encodes a 1400 amino acid protein (SEQ ID NO:42).

As assessed by TaqMan analysis, 1660 mRNA was expressed the breast, ovary, lung, colon and small intestine tumors. Oncology tissue panels showed that 1660 was expressed in a subset of breast and colon tumors at higher levels than the respective normal tissue samples. Breast cancer tissue panels showed that 1660 was expressed in primary breast tumors at higher levels than normal breast samples. Colon cancer tissue panel showed that 1660 was expressed in stage C primary colon tumors and colon liver metastases in comparison to stage B tumors and normal colon tissues.

1660 or (RON) functions as an oncogene by activating the beta-catenin pathway, as cells transfected with 1660 have increased levels of phosphorylated beta-catenin, c-myc and cyclin D1 (Dnilkovitch-Miagkova et al., 2001). Published data has shown that 1660 is a receptor tyrosine kinase that is expressed at high levels in 50% of primary breast carcinomas (Maggiora et al., 1998). 1660 expression has also been shown in colon epithelial cells also protected against apoptotic death (Chen et al., 2000). Due to the increased expression of 1660 mRNA in a number of human tumors and its functional role, modulators of 1660 activity would be useful in treating cancer. 1660 polypeptides of the present invention would be useful in screening for modulators of 1660 activity.

Gene ID 1450

The human 1450 sequence (SEQ ID NO:43), known also as Fibroblast Growth Factor Receptor 4 Precursor (FGFR-4), is approximately 2915 nucleotides long including untranslated regions. The coding sequence, located at about nucleic acids 56 to 2464 of SEQ ID NO:43, encodes a 802 amino acid protein (SEQ ID NO:44).

TaqMan analysis showed 1450 mRNA expression in colon, lung, pancreas, liver, kidney was upregulated in the tumorigenic cell lines as compared to the non-tumorigenic cell lines. Oncology tissue panels showed 1450 mRNA expression in a subset of breast, colon and lung tumors. The colon liver metastasis samples also expressed much higher levels of 1450 mRNA than normal liver tissues. In ISH experiments, breast primary tumors, a subset of breast metastases, colon primary tumors and colon metastases to the liver were positive for 1450. One lung adenocarcinoma expressed 1450. No expression was observed in normal tissue for each tumor type examined.

1450 or fibroblast growth factor receptor 4 (FGFR4) is a transmembrane tyrosine kinase receptor that binds acidic FGF (Ron et al.,1993). FGFs are also involved in cell proliferation, migration and differentiation in embryonic development, and tissue repair /injury response in adults (Ornitz et al., 2001). Inappropriate expression of some FGFs contributes to cancer pathogenesis (Id.). 1450 signaling can contribute to the proliferation and increase invasive properties of tumor cells and the inhibition of 1450 will inhibit tumor progression. The increased expression of 1450 mRNA in tumors indicates that modulators of 1450 would be useful as in treating cancer. 1450 polypeptides of the present invention would be useful in screening for modulators of 1450 activity.

Gene ID 18894

The human 18894 sequence (SEQ ID NO:45), known also as an Endothelial Lipase, is approximately 3927 nucleotides long including untranslated regions. The coding sequence, located at about nucleic acids 253 to 1755 of SEQ ID NO:45, encodes a 500 amino acid protein (SEQ ID NO:46).

As assessed by TaqMan expression analysis, 18894 mRNA expression was increased in ovary, kidney, liver, and colon tumor samples as compared to their respective normal tissues. Ovarian and angiogenesis cancer cell model panels showed that 18894 was expressed in many serous ovarian tumors and clear cell tumors as wells as cervical tumors.

Endothelial lipase is an extracellular enzyme attached to the endothelial surface involved in binding and hydrolysis of lipoproteins and internalization of their products.

Binding of endothelial lipase increases the proliferation of endothelial cells, partly mediated by binding of lipoproteins inhibition of endothelial lipase would be expected to be antiangiogenic. Due to 18894 mRNA expression in angiogenic tissues, including clear cell carcinoma of the ovary, discovering modulators of 18894 activity would be useful in treating human cancers, including but not limited to cancers of the ovary, colon, kidney and liver, along with diseases characterized by aberrant angiogenesis. 18894 polypeptides of the present invention would be useful in screening for modulators of 18894 activity.

Gene ID 2088

The human 2088 sequence (SEQ ID NO:47), known also as Tousled-Like Kinase or PKU-beta, is approximately 4299 nucleotides long including untranslated regions. The coding sequence, located at about nucleic acids 213 to 2576 of SEQ ID NO:47, encodes a 787 amino acid protein (SEQ ID NO:48).

As assessed by TaqMan analysis, 2088 mRNA expression was increased in brain tumor samples as compared to the respective normal tissues. 2088 mRNA expression was also upregulated in endothelia cultures and during EC tube formation. In Oncology panels, 2088 mRNA is expressed at a relatively high level in HUVEC culture and up-regulated in prostate, colon, and lung tumors. The expression of 2088 mRNA is upregulated in 1/6 breast tumors, 6/6 lung tumors and 1/5 colon tumors as compared to normal tissues. In an Angiogenesis panel, the expression of 2088 mRNA is upregulated in human fetal tissues, 4/7 hemangiomas, 3/5 Wilm's tumors, 3/3 lung tumors, 3/3 colon tumors, and 1/5 cervix tumors as compared to adult normal tissues. 2088 mRNA expression levels are also high in glioblastomas.

ISH experiments showed upregulation of 2088 mRNA in Wilm's, cervical and lung tumor cells and associated stroma. Colon cancer cells had significantly upregulated 2088 mRNA in tumor epithelium.

2088 is a tousled-like kinase with an Asf1 homolog as a substrate. 2088 activity promotes assembly of the nucleosome on newly synthesized DNA. 2088 activity also promotes S-phase progression and inhibits the DNA replication checkpoint. Inhibition of 2088 would inhibit S-phase progression and promote the DNA replication checkpoint, preventing endothelial cell proliferation and enhancing DNA damage sensitivity of cancer cells. Due to 2088 expression in a number of human tumors, modulators of 2088 activity would be useful in treating human cancers, as well as diseases characterized by aberrant angiogenesis. 2088 polypeptides of the present invention would be useful in screening for modulators of 2088 activity.

Gene ID 32427

The human 32427 sequence (SEQ ID NO:49) known also as long chain fatty acid CoA ligase 5 (LACS5), is approximately 3371 nucleotides long including untranslated regions. The coding sequence, located at about nucleic acids 114 to 2333 of SEQ ID NO:49, encodes a 739 amino acid protein (SEQ ID NO:50).

As assessed by TaqMan analysis, 32427 mRNA was found to be upregulated in lung and colon tumors when compared to normal control tissues. As assessed by ISH, low levels of expression of 32427 mRNA are seen in the normal epithelium of ovary and lung tissues, with markedly increased expression in the epithelium of tumors derived from these tissues. 32427 mRNA showed high levels of expression in both normal and tumor epithelium in the colon.

32427 activates fatty acids for both synthesis of cellular lipids and degradation via β-oxidation. 32427 protein localizes to mitochondria and is increased during fasting, suggesting that 32427's primary role is in β-oxidation of lipids for energy production. 32427 is upregulated by kras in tumor cells under conditions in which nutrients are limiting ( i.e. unvascularized or poorly vascularized tumors). Due to 32427 mRNA expression in lung and colon tumors, along with its functional role, modulators of 32427 activity would be useful in treating human cancers, including but not limited to cancers of the lung and colon. 32427 polypeptides of the present invention are useful in screening for modulators of 32427 activity.

Gene ID 2160

The human 2160 sequence (SEQ ID NO:51), known also as MAPK-activated protein kinase (Mnk1), is approximately 2617 nucleotides long including untranslated regions. The coding sequence, located at about nucleic acids 188 to 1462 of SEQ ID NO:51, encodes a 424 amino acid protein (SEQ ID NO:52).

As assessed by TaqMan analysis, 2160 mRNA was found to be upregulated in colon and lung tumors relative to respective normal tissues. Oncology model panels showed a high level of expression of 2160 in lung tumors when compared to normal lung tissue. Colon model panels showed an increased expression of 2160 in colon metastases when compared to normal colon tissue.

2160 or Mnk1 is a serine/threonine kinase which is phosphorylated and activated by ERK1, ERK2, and p38 MAP kinases. 2160 or Mnk1 is involved in the regulation of the phosphorylation of the cap-binding protein, eIF4E. 2160 or Mnk1 is recruited by eIF4G to activate eIF4E for the translation of proteins involved in mitosis. Therefore, an increased expression of 2160 in tumors contributes to higher rates of proliferation by increasing the translation of mitotic proteins. Due to 2160 expression in a colon and lung tumors, along with its functional role, modulators of 2160 activity would be useful in treating human cancers, including but not limited to cancers of the lung and colon. 2160 polypeptides of the present invention are useful in screening for modulators of 2160 activity.

Gene ID 9252

The human 9252 sequence (SEQ ID NO:53), known also as a serine hydroxymethyl transferase (cSHMT), is approximately 1599 nucleotides long including untranslated regions. The coding sequence, located at about nucleic acids 13 to 1464 of SEQ ID NO:53, encodes a 483 amino acid protein (SEQ ID NO:54).

As assessed by TaqMan analysis, 9252 mRNA expression was shown to be increased in lung and colon tumors when compared to respective normal lung and colon tissues. Cell model panels showed transient decreased expression upon p53 activation and increased expression in ½ kras transformed cell lines. As assessed by ISH analysis, 9252 mRNA had low to moderate levels of expression in epithelium of lung and colon tumors with no expression evident in normal epithelium of the bronchiole or colon.

9252 or cSHMT is a key regulator of folate-activated one-carbon units that are required for purine, thymidine, and methionine biosynthesis. Enhanced 9252 or cSHMT expression increases the available pools of nucleotides that are required for DNA synthesis in rapidly proliferating cells. Due to 9252 expression in a lung and colon tumors, along with its functional role, modulators of 9252 activity would be useful in treating human cancers, including but not limited to cancers of the lung and colon. 9252 polypeptides of the present invention are useful in screening for modulators of 9252 activity.

Gene ID 9389

The human 9389 sequence (SEQ ID NO:55), known also as hydroxymethylglutaryl-COA synthase (HMG-COA), is approximately 1650 nucleotides long including untranslated regions. The coding sequence, located at about nucleic acids 22 to 1584 of SEQ ID NO:55, encodes a 520 amino acid protein (SEQ ID NO:56).

As assessed by TaqMan analysis, 9389 mRNA was found to be upregulated in lung and breast tumors when compared to normal control tissues. Further analysis also showed that 9389 was downregulated by p53 and upregulated by mutant kras. In addition, Her2/neu overexpression and EGF or IGF treatment both increased 9389 levels in breast cancer cells.

9389 or HMG-CoA synthase functions in the cholesterol biosynthesis pathway, converting acetyl-CoA to 3-hydroxy-3-methyl-glutaryl-CoA (HMG-CoA). Downstream products of this pathway are required for the farnesylation or geranylation of the kras protein, which in turn is strictly required for its membrane association and signaling activity. Increased expression of 9389 or HMG-CoA synthase would potentially increase the levels of active, membrane associated kras in the cell. In addition, drugs that inhibit this pathway have also shown to have anti-tumorigenic effects. Due to 9389 expression in a lung and breast tumors, along with its functional role, modulators of 9389 activity would be useful in treating human cancers, including but not limited to cancers of the lung and breast. 9389 polypeptides of the present invention are useful in screening for modulators of 9389.

Gene ID 1642

The human 1642 sequence (SEQ ID NO:57), known also as a NIMA-related protein kinase 2 (NEK2), is approximately 2051 nucleotides long including untranslated regions. The coding sequence, located at about nucleic acids 83 to 1420 of SEQ ID NO:57, encodes a 445 amino acid protein (SEQ ID NO:58).

As assessed by TaqMan analysis, 1642 mRNA was found to be upregulated in breast, ovary, lung and colon tumors compared to normal control tissues. Further breast cancer model panels showed that 1642 was upregulated in breast cancer tumors when compared to respective normal breast tissues. Further TaqMan analysis showed 1642 mRNA is expressed at increased levels in 7/7 primary ovarian tumors and 7/7 ovarian omentum metastases vs. 6/6 normal ovary tissue samples.

1642 is a human homologue of NIMA (47% identity in catalytic domains) and is also known as NIMA-like kinase1 (NLK1) or is NIMA-related kinase (NEK2). Published literature indicates that NIMA kinase activity is cell cycle regulated, with high activity at mitosis (G2/M) and low activity in S-phase (Osmani et al., 1991). 1642 or Nek2 resembles NIMA in its primary structure, cell cycle regulation and substrate specificity, suggesting that Nek2 would potentially be required for mitosis. Due to the increased expression of 1642 in transformed and tumorigenic breast epithelial cells suggests that Nek2 would be a potential therapeutic target for cancer cells. Therefore, discovering modulators of 1642 activity would be useful in treating human cancers, including but not limited to cancers of the breast, ovary, lung and colon. 1642 polypeptides of the present invention are also useful in screening for modulators of 1642 activity.

Gene ID 85269

The human 85269 sequence (SEQ ID NO:59) is approximately 1548 nucleotides long including untranslated regions. The coding sequence, located at about nucleic acids 115 to 1203 of SEQ ID NO:59, encodes a 362 amino acid protein (SEQ ID NO:60).

As assessed by TaqMan analysis, 85269 mRNA was found to be upregulated in a subset of ovarian, colon and breast tumors when compared to respective normal tissues. Oncology model panels showed and increase level of 85269 mRNA in breast and ovary tumors in comparison to respective normal tissues.

Activation or overexpression of cellular growth factors and their receptors, including EGFR and EGF/TGFalpha are implicated in the development and progression of breast cancer. Many mouse experiments demonstrate the ability of EGF and TGFalpha to promote mammary tumorigenesis, and members of this family are commonly overexpressed in human breast cancers. 85269 expression in human ovarian, colon and breast cancers suggests that 85269 plays an important role in tumor cell survival and growth. Therefore, discovering modulators of 85269 activity would be useful in identifying potential targets for therapeutic intervention in cancers, including but not limited to cancers of the ovary, colon and breast. In addition, 85269 polypeptides of the present invention are useful in screening for modulators of 85269 activity.

Gene ID 10297

The human 10297 sequence (SEQ ID NO:61) known also as purine nucleoside phosphorylase (PNP), is approximately 1418 nucleotides long including untranslated regions. The coding sequence, located at about nucleic acids 110 to 979 of SEQ ID NO:61, encodes a 289 amino acid protein (SEQ ID NO:62).

As assessed by TaqMan analysis, 10297 was shown to be upregulated in breast, ovary, lung and colon tumors when compared to normal control tissues. Further analysis showed that 10297 was upregulated in fetal tissue and angiogenic tumors. 10297 expression in a breast, ovary, lung and colon tumors suggest a role of 10297 in angiogenesis and tumorigenesis. Therefore, discovering modulators of 10297 activity would be useful in treating human cancers, including but not limited to cancers of the breast, ovary, lung and colon, and conditions characterized by aberrant angiogenesis. In addition, 10297 polypeptides of the present invention would also be useful in screening for modulators of 10297 activity.

Gene ID 1584

The human 1584 sequence (SEQ ID NO:63) known also as a serine/threonine kinase PCTAIRE-1, is approximately 1745 nucleotides long including untranslated regions. The coding sequence, located at about nucleic acids 124 to 1614 of SEQ ID NO:63, encodes a 496 amino acid protein (SEQ ID NO:64).

As assessed by TaqMan analysis, 1584 mRNA was found to be upregulated in breast, ovary, lung and colon tumors. Further analysis also showed 1584 mRNA to be upregulated in proliferating EC and EC tube formation, fetal tissue, hemangiomas and angiogenic tumors. 1584 expression in a breast, ovary, lung and colon tumors suggest a role of 1584 in angiogenesis and tumorigenesis. Therefore, discovering modulators of 1584 activity would be useful in treating human cancers, including but not limited to cancers of the breast, ovary, lung and colon, and in conditions characterized by aberrant angiogenesis. In addition, 1584 polypeptides of the present invention would also be useful in screening for modulators of 1584 activity.

Gene ID 9525

The human 9525 sequence (SEQ ID NO:65), known also as a diacylglycerol kinase, is approximately 6207 nucleotides long including untranslated regions. The coding sequence, located at about nucleic acids 81 to 3590 of SEQ ID NO:65, encodes a 1169 amino acid protein (SEQ ID NO:66).

As assessed by TaqMan analysis, 9525 mRNA was upregulated in fetal tissue, hemangiomas and angiogenic tumors when compared to normal control tissues. Further TaqMan analysis indicated that 9525 mRNA was up regulated in endothelial tube (EC) formation.

9525 is a diacylglycerol kinase (DGK). DGKs regulate the levels of two important signaling molecules, diacylglycerol and phosphatidic acid. Phosphatidic acid is a mitogenic phospholipid that interacts directly with Raf-1, recruiting it to the membrane and allowing it to interact with Ras. Tumor cells upregulate DGKd to maximize the mitogenic signal through the Ras pathway. Inhibition of DGKd will downregulate c-myc, c-fos, c-raf, and cyclin D3, resulting in inhibition of proliferation of tumor cells. Due to 9525 mRNA expression in fetal tissue, hemangiomas and angiogenic tumors, along with its functional role, modulators of 9525 activity would be useful in treating human cancers. 9525 polypeptides of the present invention are useful in screening for modulators of 9525 activity.

Gene ID 14124

The human 14124 sequence (SEQ ID NO:67), known also as Cyclin G-associated kinase (HsGAK), is approximately 4331 nucleotides long including untranslated regions. The coding sequence, located at about nucleic acids 1 to 3936 of SEQ ID NO:67, encodes a 1311 amino acid protein (SEQ ID NO:68).

As assessed by TaqMan analysis, 14124 mRNA was upregulated in breast, lung and colon tumors when compared to normal control tissues. Further TaqMan analysis showed that 14124 mRNA was upregulated in fetal tissue, hemangiomas and angiogenic tumors. 14124 mRNA was also upregulated in proliferating endothelial cell (EC) and EC tube formation. ISH expression analysis confirms the TaqMan results in lung, colon, fetal and angiogenic tissues. The expression patterns of 14124 indicate a potential role of 14124 in tumorigenesis and angiogenesis. Due to 14124 mRNA expression in breast, lung and colon tumors, along with its functional role, modulators of 14124 activity would be useful in treating human cancers, including but not limited to cancers of the breast, lung and colon. 14124 polypeptides of the present invention are useful in screening for modulators of 14124 activity.

Gene ID 4469

The human 4469 sequence (SEQ ID NO:69), known also as HSNFRK, is approximately 5519 nucleotides long including untranslated regions. The coding sequence, located at about nucleic acids 642 to 2939 of SEQ ID NO:69, encodes a 765 amino acid protein (SEQ ID NO:70).

As assessed by TaqMan analysis, 4469 mRNA was upregulated breast, lung, and prostate tumors when compared to normal control tissues. Further TaqMan analysis showed that 4469 mRNA was upregulated in fetal tissue, hemangiomas and angiogenic tumors. 4469 mRNA was upregulated in proliferating endothelial cells (EC), EC tube formation, growth factor-treated ECs and endothelial culture. 4469 mRNA was also upregulated in hypoxia treated human microvascular endothelial cells (HMVEC). As assessed by ISH analysis, 4469 mRNA was upregulated in colon, lung and ovary tumor samples. It also showed that 4469 mRNA is expressed in endothelial cells of hemangiomas, Wilm's tumors, renal cell carcinoma, and fetal adrenal. In the Wilm's tumors and fetal adrenal samples, the cells expressing 4469 mRNA are epithelial cells and cells in vessels.

The expression patterns of 4469 indicate a potential role of 4469 in tumorigenesis and angiogenesis. Due to 4469 mRNA expression in breast, lung, and prostate tumors, along with its functional role, modulators of 4469 activity would be useful in treating human cancers, including but not limited to cancers of the breast, lung, and prostate. 4469 polypeptides of the present invention are useful in screening for modulators of 4469 activity.

Gene ID 8990

The human 8990 sequence (SEQ ID NO:71), known also as Non-hepatic arginase (Kidney-type arginase) or arginase type II, is approximately 1354 nucleotides long including untranslated regions. The coding sequence, located at about nucleic acids 21 to 1085 of SEQ ID NO:71, encodes a 354 amino acid protein (SEQ ID NO:72).

As assessed by TaqMan analysis, 8990 mRNA was upregulated in lung tumors when compared to normal control tissues.

The pro-apoptotic action of nitric oxide synthase inhibitors (like NG-monomethyl-L-arginine) is manifested through inhibition of the arginase pathway. These inhibitors cause apoptosis in cell cultures and inhibit the growth of various transplantable tumors (Szende B et al 2001 Cancer Cell Int.Dec 17; 1(1):3). N-hydroxy-L-argenine (NOHA), an intermediate in the nitric oxide synthetic pathway and an inhibitor of arginase, significantly reduces intracellular polyamines, activates caspase-s, and induces apoptosis in the breast cancer cell line (MDA-MB-468) (Singh R, et al 2001 Carcinogenesis November; 22(11): 1986-9). 8990 or arginase is the first step in arginine degradation in the urea cycle. Therefore, 8990 or arginase plays a potential role in the regulation of extra-urea cycle, arginine metabolism, and down-regulation of nitric oxide synthesis. Due to 8990 mRNA expression in lung tumors, along with its functional role, modulators of 8990 activity would be useful in treating human cancers including but not limited to cancers of the lung. 8990 polypeptides of the present invention are useful in screening for modulators of 8990 activity.

Gene ID 2100

The human 2100 sequence (SEQ ID NO:73), known also as a protein kinase C-like 1 (PKL1), is approximately 2988 nucleotides long including untranslated regions. The coding sequence, located at about nucleic acids 85 to 2913 of SEQ ID NO:73, encodes a 942 amino acid protein (SEQ ID NO:74).

As assessed by TaqMan analysis, 2100 mRNA was upregulated in breast, colon and liver tumors when compared to normal control tissues. Further TaqMan analysis indicated that 2100 mRNA had regulated expression in ovary and colon tumors, as well as, colon, omentum and fallopian tube metastases when compared to normal control tissues. Further TaqMan analysis also indicated that substantially higher expression of 2100 mRNA was detected in colon metastases compared to normal colon and liver. There was increased expression of 2100 mRNA detected in poorly differentiated non-small cell lung carcinomas compared to normal and adenocarcinoma lung tissue and decreased expression of 2100 mRNA was detected in the p16 induction model and the p53 induction model, relative to uninduced controls and vector-transfected controls. As assessed by ISH analysis, 2100 mRNA is expressed in primary tumors of the colon, breast, lung, and ovary. Lung tumor expression is most abundant in small cell lung carcinoma. Colon metastases to the liver also show expression, but to a lesser degree than primary colon tumors. Normal tissues show some expression in ovarian tissue, but normal lung, colon, and breast are negative for 2100 mRNA expression.

2100 is a protein kinase C-like 1 (PKL1). 2100 mediates GTPase Rho-dependent signaling. 2100 is upregulated in colon tumor samples and higher expression may favor growth and survival. Selective inhibition of 2100 results in reduced survival of tumor cells. Due to 2100 mRNA expression in breast, colon and liver tumors, along with its functional role, modulators of 2100 activity would be useful in treating human cancers, including but not limited to cancers of the breast, colon and liver. 2100 polypeptides of the present invention are useful in screening for modulators of 2100 activity.

Gene ID 9288

The human 9288 sequence (SEQ ID NO:75), known also as fumarylacetoacetate hydrolase (FAH), is approximately 1447 nucleotides long including untranslated regions. The coding sequence, located at about nucleic acids 57 to 1316 of SEQ ID NO:75, encodes a 419 amino acid protein (SEQ ID NO:76).

As assessed by TaqMan analysis, 9288 mRNA was expressed at highest levels in erythroid cells, adipose and liver tissues. Further TaqMan analysis showed increased expression in a colon tumor pool relative to the normal colon tissue pool. Oncology tissue panels indicated that 9288 mRNA was expressed at high levels in three breast tumor samples (IDC-MD/PD, IDC-PD and ILC), all of which expressed the highest levels of the oncogenic receptor tyrosine kinase (Her-2). Breast cancer tissue panels indicated that 9288 was overexpressed in more than half of the primary breast tumor samples when compared to normal control tissues. In addition, 9288 mRNA was expressed at high levels in ZR-75 cells in breast cancer cell model panels. Her-2 cell model panels, indicated that 9288 was up regulated in MCF10A single clones which overexpress the wild type oncogenic receptor tyrosine kinase (Her-2).

9288 or Fumarylacetoacetate hydrolase (FAH) is the enzyme deficient in hereditary tyrosimenia type I patients (HT1) (Tanguay, R. M., et al., 1996, Tyrosine and its catabolites: from disease to cancer, Acta Biochim Pol 43(1):209-216; Jorquera, R. and R. M. Tanguay, 2001, Rumarylacetoacetate, the metabolite accumulating in hereditary tyrosinemia, activates the ERK pathway and induces mitotic abnormalities and genomic instability, Hum Mol Genet 10(17): 1741-1752). 9288 catalyzes the reaction 4-fumarylacetoacetate (FAA)+ water to acetoacetate+ fumarate. 9288 is involved in the last step in the degradation of the amino acid tyrosine. In the absence of 9288, the mutagenic metabolite fumarylacetoacetate (FAA) accumulates. Fumarylacetoacetate (FAA) is responsible for the increased incidence of hepatocellular carcinoma associated with HT1 patients (Tanguay, R. M., et al., 1996, Tyrosine and its catabolites: from disease to cancer, Acta Biochim Pol 43(1):209-216). In addition, fumarylacetoacetate causes organelle/mitotic spindle disturbances that contributes to genetic instability (Jorquera, R. and R. M. Tanguay, 2001, Rumarylacetoacetate, the metabolite accumulating in hereditary tyrosinemia, activates the ERK pathway and induces mitotic abnormalities and genomic instability, Hum Mol Genet 10(17): 1741-1752). The increase in cancer due to 9288 or fumarylacetoacetate hydrolase (FAH) loss is attributed to cumulative mutagenic effects on FAH deficient individuals through time.

Another enzyme that is involved in regulating intracellular fumarate levels is fumarate hydratase (FH), a mitochondrial enzyme catalyzing the seventh step of citric acid cycle. Fumarate hydratase (FH) catalyzes L-malate formation from fumarate hydration. Published literature describes the mapping of fumarate hydratase (FH) as the gene with germline mutations that predisposes individuals to uterine fibroids, skin leiomyomata and papillary renal cell cancer (Tomlinson, I. P., et al, 2002, Germline mutations in FH predispose to dominantly inherited uterine fibroids, skin leiomyomata and papillary renal cell cancer, Nat Genetics 30(4): 402-410). Fumarate hydratase (FH) mutations in these individuals results in low or absent FH activity in their tumors. Loss of fumarate hydratase (FH) function causes an accumulation of fumarate leading to tumor formation (Rustin, P., 2002, Mitochondria, from cell death to proliferation, Nat Genetics 30(4): 352-353). Therefore, increased 9288 or fumarylacetoacetate hydrolase (FAH) activity leads to an accumulation of fumarate and subsequent tumor formation. Inhibition of 9288 or fumarylacetoacetate hydrolase (FAH) activity decreases fumarate levels and its tumor promoting effects. Due to 9288 mRNA expression in erythroid cells, adipose tissue and liver, along with its functional role, modulators of 9288 activity would be useful in treating human cancers. 9288 polypeptides of the present invention are useful in screening for modulators of 9288 activity.

Gene ID 64698

The human 64698 sequence (SEQ ID NO:77), known also as a sphingosine kinase 2 (SPK2), is approximately 2380 nucleotides long including untranslated regions. The coding sequence, located at about nucleic acids 7 to 1863 of SEQ ID NO:77, encodes a 618 amino acid protein (SEQ ID NO:78).

As assessed by TaqMan analysis, 64698 mRNA was upregulated in brain, erythroid cells, breast and lung tumors. Further TaqMan analysis indicated that 64698 mRNA was increased two-fold in the colon tumor pool when compared to normal colon tissues. Expanded lung cancer tissue panels, indicated that 64698 mRNA was expressed at moderate to low levels in many lung tumors. Expanded ovarian cancer xenograft panels, indicated that 64698 mRNA was expressed at highest levels in plastic-grown SKOV3 cells. Ovarian cell model panels, indicated that 64698 mRNA was increased in SKOV3 variant cells when compared with SKOV3WT cells. In addition, Oncology xenograft cell line panels indicated that 64698 mRNA was expressed at high levels in most tumor cell lines.

The conversion of ceramide into sphingosine and sphingosine 1-phosphate (SIP) determines cell fate. Published data indicates ceramide induces apoptosis (caspase activation), whereas SIP induces cell survival. 64698 or Sphingosine kinases (SPK2), catalyzes the phosphorylation of sphingosine to sphingosine 1-phosphate. Overexpression of sphingosine kinase 1 (SPK2), in NIH3T3 cells and HEK293 cells promotes cell growth in low serum medium, the G1/S transition, DNA synthesis, and increased cell number (Pyne, S. and N. J. Pyne, 2000, Sphingosine 1-phosphate signaling in mammalian cells, Biochem J. 349: 385-402). Overexpression of 64698, also protects against apoptosis induced by serum deprivation, ceramide, or anti-Fas. Importantly, the 64698 inhibitor, N N-dimethylsphingosine, blocks the protective effect of overexpression. These effects are a result of the extracellular and intracellular roles of S1P or by preventing the accumulation of proapoptotic ceramide (Pyne, S. and N. J. Pyne, 2000, Sphingosine 1-phosphate signaling in mammalian cells, Biochem J. 349: 385-402.). In addition, overexpression of 64698, can transform cells in vitro causing them to become tumorigenic in immunocompromised mice (Xia, P., et. al., 2000, An oncogenic role of sphingosine kinase, Curr. Biol. 10(23): 1527-1530).

Tumor cells upregulate 64698 or sphingosine kinase 2 to reduce the cellular pool of sphingosine available as a substrate for ceramidase. Increased production of sphingosine I-phosphate promotes cell survival via its role as a ligand to EDG receptors and some possible intracellular roles. Inhibition of sphingosine kinase 2 allows for inherent apoptotic signals to increase ceramide levels, Due to 64698 mRNA expression in brain, erythroid cells, breast and lung tumors, along with its functional role, modulators of 64698 activity would be useful in treating human cancers, including but not limited to cancers of the brain, breast, lung and colon. 64698 polypeptides of the present invention are useful in screening for modulators of 64698 activity.

Gene ID 10480

The human 10480 sequence (SEQ ID NO:79), known also as Succinyl CoA:3-oxoacid CoA-transferase, is approximately 3337 nucleotides long including untranslated regions. The coding sequence, located at about nucleic acids 99 to 1661 of SEQ ID NO:79, encodes a 520 amino acid protein (SEQ ID NO:80).

As assessed by TaqMan analysis, 10480 mRNA showed broad expression in normal tissues. 10480 mRNA showed increased expression in 6/6 lung tumors when compared to normal tissues with no increase seen in other tumor types. 10480 mRNA also showed late downregulation in cells expressing active p53. 10480 mRNA was also identified in immortalized bronchial epithelial cells transformed with mutant kras.

10480 or Succinyl-CoA:3-ketoacid-CoA transferase (SCOT) functions as the first step in catabolism of ketone bodies that are formed by β-oxidation of fatty acids. A number of lines of evidence from profiling experiments point to increased use of fatty acids as an energy source by tumor cells, which presumably results in higher levels of ketone bodies within these cells. SCOT may be increased in tumor cells as a mechanism for metabolizing ketone bodies, which can be detrimental to cells at high concentrations.

Due to 10480 mRNA expression in the lung, along with its functional role, modulators of 10480 activity would be useful in treating human cancers, including but not limited to cancers of the lung. 10480 polypeptides of the present invention are useful in screening for modulators of 10480 activity.

Gene ID 20893

The human 20893 sequence (SEQ ID NO:81) known also as a serine/threonine-protein kinase, is approximately 6828 nucleotides long including untranslated regions. The coding sequence, located at about nucleic acids 1381 to 3366 of SEQ ID NO:81, encodes a 661 amino acid protein (SEQ ID NO:82).

As assessed by TaqMan analysis, 20893 mRNA was upregulated in lung tumors when compared to normal control tissues. Further TaqMan analysis indicated that 20893 mRNA expression increased in cell expressing activated kras. 20893 mRNA was also identified in immortalized bronchial epithelial cells transformed with mutant kras.

20893 is an uncharacterized kinase that shows elevated expression in lung tumors and whose expression is increased by activation of the kras oncogene. Given the above expression characteristics, 20893, plays a potential role in cellular proliferation. Due to 20893 mRNA expression in lung tumors, along with its functional role, modulators of 20893 activity would be useful in treating human cancers, including but not limited to cancers of the lung. 20893 polypeptides of the present invention are useful in screening for modulators of 20893 activity.

Gene ID 33230

The human 33230 sequence (SEQ ID NO:83) known also as Acetyl CoA synthetase (ACS), is approximately 3608 nucleotides long including untranslated regions. The coding sequence, located at about nucleic acids 1 to 2070 of SEQ ID NO:83, encodes a 689 amino acid protein (SEQ ID NO:84).

As assessed by TaqMan analysis, 33230 mRNA was expressed in lung and colon tumors when compared to normal control tissues. Further TaqMan experiments in expanded lung and colon panels indicated that 33230 mRNA was expressed in non-small cell carcinomas.

33230 or ACS catalyzes the activation of acetate, a reaction that is essential for lipid synthesis and energy generation. A pattern of regulation of 33230 by sterol regulatory element-binding proteins (SREBPs) resembles that of enzymes of fatty acid synthesis. Published literature indicates that 33230 expression is substantially elevated in tumor metastases in comparison to normal tissues. Therefore, selective inhibition of 33230 prevents metastatic growth of cancer cells. Due to 33230 mRNA expression in lung and colon tumors, along with its functional role, modulators of 33230 activity would be useful in treating human cancers, including but not limited to cancers of the lung and colon. 33230 polypeptides of the present invention are useful in screening for modulators of 33230 activity.

Gene ID 1586

The human 1586 sequence (SEQ ID NO:85), known also as PCTAIRE 2, is approximately 1738 nucleotides long including untranslated regions. The coding sequence, located at about nucleic acids 70 to 1641 of SEQ ID NO:85, encodes a 523 amino acid protein (SEQ ID NO:86).

As assessed by TaqMan analysis, 1586 mRNA was upregulated in lung tumors when compared to normal lung tissues. Further TaqMan analysis indicated that 1586 mRNA was also expressed in Beas-2B cells expressing mutant kras.

1586 or PCTAIRE 2 is a member of a subfamily of Cdc2-related kinases. Cdc2-related kinases have been implicated in both cell cycle control (Charrasse S. et al. Cell Growth Differ Septemer 1999; 10(9):611-20) and cellular differentiation (Hirose, T. et al. Eur J Biochem Oct. 15, 1997;249(2):481-8). The striking increase in 1586 expression levels in human tumors coupled with its upregulation by mutant kras in bronchial epithelial cell lines make it a potential player in lung tumor progression. Due to 1586 mRNA expression in the lung, along with its functional role, modulators of 1586 activity would be useful in treating human cancers including but not limited to cancers of the lung. 1586 polypeptides of the present invention are useful in screening for modulators of 1586 activity.

Gene ID 9943

The human 9943 sequence (SEQ ID NO:87), known also as a calcium/calmodulin-dependent protein kinase type II delta chain (CaMK-II), is approximately 1500 nucleotides long including untranslated regions. The coding sequence, located at about nucleic acids 1 to 1500 of SEQ ID NO:87, encodes a 499 amino acid protein (SEQ ID NO:88).

As assessed by TaqMan analysis, 9943 mRNA was upregulated in clinical lung tumors when compared to normal lung tissue. Further TaqMan analysis indicated that 9943 mRNA was upregulated in cells expressing an inducible form of active kras. 9943 mRNA was also downregulated by p53.

Calcium/calmodulin dependent kinases are major effectors of Ca²⁺ signaling pathways. The phenotypic effect of 9943 or CaMK-II signaling has been implicated in both cell cycle progression (Morris, T A. Exp Cell Res May 1, 1998;240(2):218-27) and cellular differentiation. Increased 9943 or CaMK-II activity in the context of a tumor cells enables ready transition through the G1 checkpoint and increase cellular proliferation. Due to 9943 mRNA expression in lung tumors, along with its functional role, modulators of 9943 activity would be useful in treating human cancers, including but not limited to cancers of the lung. 9943 polypeptides of the present invention are useful in screening for modulators of 9943 activity.

Gene ID 16334

The human 16334 sequence (SEQ ID NO:89), known also as intestinal cell kinase, is approximately 6014 nucleotides long including untranslated regions. The coding sequence, located at about nucleic acids 267 to 2165 of SEQ ID NO:89, encodes a 632 amino acid protein (SEQ ID NO:90).

As assessed by TaqMan analysis, 16334 mRNA was upregulated in breast, lung and colon tumors. Further TaqMan analysis indicated that 16334 mRNA was upregulated in endothelial cell (EC) tube formation, endothelial culture, fetal and angiogenic tissues.

The expression patterns of 16334 indicate a role for 16334 in tumorigenesis and/or angiogenesis. Due to 16334 mRNA expression in breast, lung, and colon tumors, along with its functional role, modulators of 16334 activity would be useful in treating human cancers, including but not limited to cancers of the breast, lung, and colon. 16334 polypeptides of the present invention are useful in screening for modulators of 16334 activity.

Gene ID 68862

The human 68862 sequence (SEQ ID NO:91), known also as a pkinase, is approximately 2980 nucleotides long including untranslated regions. The coding sequence, located at about nucleic acids 86 to 2422 of SEQ ID NO:91, encodes a 778 amino acid protein (SEQ ID NO:92).

As assessed by TaqMan analysis, 68862 mRNA was upregulated in breast and lung tumors. Further TaqMan experiments indicate that 68862 mRNA was upregulated in fetal tissues and angiogenic tumors. As assessed by ISH, 68862 mRNA was upregulated in lung tumors as well as angiogenic tumors and fetal tissues.

The expression patterns of 68862 indicate a role of 68862 in tumorigenesis and/or angiogenesis. Due to 68862 mRNA expression in breast and lung tumors, along with its functional role, modulators of 68862 activity would be useful in treating human cancers, including but not limited to cancers of the breast and lung. 68862 polypeptides of the present invention are useful in screening for modulators of 68862 activity.

Gene ID 9011

The human 9011 sequence (SEQ ID NO:93) known also as an glycosylasparaginase, is approximately 1048 nucleotides long including untranslated regions. The coding sequence, located at about nucleic acids 4 to 1044 of SEQ ID NO:93, encodes a 346 amino acid protein (SEQ ID NO:94).

As assessed by TaqMan analysis, 9011 mRNA was expressed in lung and colon cancer samples when compared normal tissue samples. Further TaqMan analysis indicated that 9011 mRNA was also upregulated in liver and colon metastasis. 9011 mRNA was also downregulated by transient overexpression of p53 both mutant and wild-type p53 cell backgrounds.

Asparagine-linked oligosaccharides are required for tumor cell invasion and metastasis (Cancer Cells 1989, 1, 87-92; Cancer Surv 1988, 7, 573-95). Aspartylglycosaminuria is an autosomal recessive lysosomal storage disease caused by the defective degradation of asparagine-linked glycoproteins by glycosylasparaginase (Biochim Biophys Acta 1999, 1455, 139-54). Prolonged inhibition of lysosomal function results in apoptosis in neuroblastoma cells (Int J Cancer 2002, 97, 775-9). Tumor cells upregulate glycosylasparaginase in order to process the greater flux of asparagine-linked glycoproteins. Inhibition of glycosylasparaginase results in reduction of lysosomal function in tumor cells due to the accumulation of Asn-GlcNAc, resulting in apoptosis. Due to 9011 mRNA expression in lung and colon tumors, along with its functional role, modulators of 9011 activity would be useful in treating human cancers, including but not limited to cancers of the lung and colon. 9011 polypeptides of the present invention are useful in screening for modulators of 9011 activity.

Gene ID 14031

The human 14031 sequence (SEQ ID NO:95) known also as serine palmitoyltransferase 1, is approximately 1621 nucleotides long including untranslated regions. The coding sequence, located at about nucleic acids 1 to 1422 of SEQ ID NO:95, encodes a 473 amino acid protein (SEQ ID NO:96).

As assessed by TaqMan analysis, 14031 mRNA was expressed in lung, colon and liver metastases when compared with normal control tissues. Further TaqMan analysis on colon oncogenes and tumor suppressor panels indicated that 14031 mRNA expression decreased in response to transient expression of p53 in DLD1 and NCM425 cell lines.

TaqMan analysis on lung cell panels indicated that 14031 mRNA expression decreased in response to stable ERp53 expression, and decreasing even further with tamoxifen activation over 3 days. TaqMan experiments on xenograft friendly cell lines indicated that 14031 mRNA was expressed in all cancer cell lines tested, especially in DLD1 colon.

14031 encodes serine palmitoyltransferase 1, the rate-limiting and committed step in de novo sphingolipid biosynthesis, including ceramide, sphingomyelins, and glycosphingolipids. Although several of the sphingolipids are growth inhibitory and induce apoptosis, several are known to be mitogenic and inhibit apoptosis, particularly the phosphosphingosines sphinganine 1-phosphate, ceramide 1-phosphate, and sphingosine 1-phosphate (J Biol Chem 2002 277, 25843-6; Biochemistry 2001 40, 4893-903). Transformed human fibroblasts have high levels of sphingomyelin synthase (SMS). High ceramide formation is fed into SMS, resulting in the formation of diacylglycerol (DAG) and activation of NF-kB (J Biol Chem 2000 275, 14760-6). Tumor cells upregulate 14031 to provide increased production of sphingolipids, which provide mitogenic and antiapoptotic signals. Therefore, inhibition of 14031 would interfere with these signals, reducing tumor growth and possibly promoting apoptosis. Due to 14031 mRNA expression in lung, colon and liver tumors, along with its functional role, modulators of 14031 activity would be useful in treating human cancers, including but not limited to cancers of the lung and colon. 14031 polypeptides of the present invention are useful in screening for modulators of 14031 activity.

Gene ID 6178

The human 6178 sequence (SEQ ID NO:97) known also as UDP-N-acetylhexosamine pyrophosphorylase (AGX) or UDPGlcNAc pyrophosphorylase (UAP), is approximately 2279 nucleotides long including untranslated regions. The coding sequence, located at about nucleic acids 233 to 1750 of SEQ ID NO:97, encodes a 505 amino acid protein (SEQ ID NO:98).

As assessed by TaqMan analysis, 6178 mRNA expression was upregulated in lung and breast tumor samples when compared normal control tissue samples. Further TaqMan analysis indicated that 6178 mRNA was upregulated upon induction of a mutant kras gene in Beas-2B cells.

6178 or UDPGlcNAc pyrophosphorylase (UAP) catalyzes the production of UDPGlcNAc in the cell cytoplasm. UDPGlcNAc is a key precursor of glycosylphosphatidylinositol (GPI), and is required for the N— and O-linked glycosylations involved in the synthesis of cell surface glycoproteins. 6178 shows consistent increased expression in cells transformed with the kirsten ras oncogene, indicating that 6178 plays a role in tumorigenesis. Therefore, increasing 6178 expression potentially augments the differential expression of cell surface glycoproteins (ex. CD44, mucin, CEA) that is seen in many tumor types. In addition, differential glycosylation of cell surface proteins in tumors is implicated in invasion, metastasis, and evasion of immune response. Due to 6178 mRNA expression in lung and breast tumors, along with its functional role, modulators of 6178 activity would be useful in treating human cancers, including but not limited to cancers of the lung and breast. 6178 polypeptides of the present invention are useful in screening for modulators of 6178 activity.

Gene ID 21225

The human 21225 sequence (SEQ ID NO:99), a probable FAD synthetase, is approximately 1740 nucleotides long including untranslated regions. The coding sequence, located at about nucleic acids 177 to 1649 of SEQ ID NO:99, encodes a 490 amino acid protein (SEQ ID NO:100).

As assessed by TaqMan analysis, 21225 mRNA was upregulated in the breast, ovary, colon and lung tumors when compared with normal control tissues. Further TaqMan analysis indicated that 21225 mRNA was upregulated in human glioblastomas, angiogenic tumor islets in RIP-Tag mouse model and E10.5 mouse yolk sac. 21225 mRNA was also up-regulated in fetal tissues, angiogenic tumors, proliferating epithelial cells (EC) and matrigel EC tubes. In situ Hybridization experiments indicated that 21225 mRNA was expressed in angiogenic tissues and tumors.

21225 mRNA encodes a probable FAD synthetase. FAD is a key cofactor is several survival and growth promoting pathways. Therefore, inhibiting 21225 will hamper FAD-dependent reactions, reducing tumor growth and increasing tumor apoptosis. Due to 21225 mRNA expression in breast, ovary, colon and lung tumors, angiogenic tissues, proliferating EC and EC tubes, along with its functional role, modulators of 21225 activity would be useful in treating human cancers, including but not limited to cancers of the breast, ovary, colon and lung. 21225 polypeptides of the present invention are useful in screening for modulators of 21225 activity.

Gene ID 1420

The human 1420 sequence (SEQ ID NO:101) known also as a Ephrin type-A receptor 1 precursor (EPHA1), is approximately 3370 nucleotides long including untranslated regions. The coding sequence, located at about nucleic acids 94 to 3048 of SEQ ID NO:101, encodes a 984 amino acid protein (SEQ ID NO:102).

As assessed by TaqMan analysis, 1420 mRNA was expressed in the ovary, lung and breast tumors (single sample pools of three tumors each), while respective normal tissues showed no 1420 mRNA expression. In addition, a single sample pool of three colon tumors each expressed the highest levels of 1420 mRNA, while normal colon expressed very low amounts of 1420 mRNA. A liver fibrosis sample also showed some expression of 1420 mRNA. Further TaqMan analysis on oncology tissue panels indicated that 1420 mRNA was expressed at increased levels in 2/5 breast tumors vs. 3/3 normal breast samples; 6/6 ovarian tumors vs. 3/3 normal ovary samples; 5/5 lung tumors vs. 4/4 normal lung samples; 3/4 colon tumors vs. 3/3 normal colon samples. TaqMan analysis on the ExBCa panel indicated that 1420 mRNA was expressed in all breast tumor samples, however two of the three normal breast samples on this panel also expressed equivalent levels of 1420 mRNA. TaqMan experiments completed on the ExCCa panel indicated that 1420 mRNA was expressed at an increased level in 5/12 colon tumors vs. 6/6 normal colon samples. In addition, 1420 mRNA was not uniformly expressed at an increased level in liver metastasis; 4/4 colon metastasis to liver had increased expression when compared to 3/6 normal liver samples. TaqMan experiments completed on the ExLCa panel indicated that 1420 mRNA was sporadically expressed in tumors (5/20 lung tumors) with infrequent overexpression when compared to normal lung tissues.

Eph-ephrin complexes control the directional movement of cells and neuronal growth cones and the establishment of the embryonic body plan. Eph receptor function is important in neurons, neural crest cells, and endothelial cells and angiogenesis. 1420 or Ephrin A1 is induced in HUVECs by TNFa. 1420 or Ephrin A1 does not cause endothelial cell proliferation but acts as chemoattractant for endothelial cells. Published literature indicates that overexpressing 1420 or EphA1 in NIH3T3 cells results in soft agar foci and tumor growth in nude mice.

Generally, overexpression of Eph receptors favor decreased cell adhesion, increased cell motility, and a higher degree of tissue invasiveness and metastasis. 1420 or EphA1 in particular is transforming and/or tumorigenic. Alternatively, secreted ephrins potentially function as diffusible chemoattractants for endothelial cells and tumor expressed Eph receptors potentially functions as contact-dependent organizing molecules to appropriately guide the incoming vessels. Due to 1420 mRNA expression in breast, ovary, colon and lung tumors, along with its functional role, modulators of 1420 activity would be useful in treating human cancers, including but not limited to cancers of the breast, ovary, colon and lung. 1420 polypeptides of the present invention are useful in screening for modulators of 1420 activity.

Gene ID 32236

The human 32236 sequence (SEQ ID NO:103) known also as long-chain acyl-CoA synthetase 6 (LACS6), is approximately 2555 nucleotides long including untranslated regions. The coding sequence, located at about nucleic acids 115 to 2208 of SEQ ID NO:103, encodes a 697 amino acid protein (SEQ ID NO:104).

As assessed by TaqMan analysis, 32236 mRNA was expressed in colon tumors (single sample pools of three tumors each), while respective normal tissues showed no 32236 mRNA expression. Brain and erythroid samples showed high expression of 32236 mRNA. Further TaqMan analysis on oncology tissue panels indicated that 32236 mRNA was expressed in 2/3 colon tumors while 3/3 normal colon samples showed no 32236 mRNA expression. TaqMan experiments completed on the ExCCa panel indicated that 32236 mRNA was expressed at an increased level in 7/12 primary colon tumors vs. 6/6 normal colon samples. In addition, 3/5 colon metastasis to liver had increased expression when compared to 5/5 normal liver samples, which had little or no expression. Further TaqMan experiments on the colon metastases to liver panel indicated that 32236 mRNA was expressed in 3/4 primary colon tumors and 9/16 colon metastases to liver with no expression normal colon and liver tissues. Furthermore, there was expression in 5/14 colon metastases to other tissues. Expression of 32236 mRNA is reduced in DLD1 cells with the transient expression of p53 or with a knocked out activated kras allele.

Due to 32236 mRNA expression in colon tumors, modulators of 32236 activity would be useful in treating human cancers, including but not limited to colon cancer. 32236 polypeptides of the present invention are useful in screening for modulators of 32236 activity.

Gene ID 2099

The human 2099 sequence (SEQ ID NO:105) known also as protein kinase C-like kinase 2 (PKL2), is approximately 3255 nucleotides long including untranslated regions. The coding sequence, located at about nucleic acids 10 to 2964 of SEQ ID NO:105, encodes a 984 amino acid protein (SEQ ID NO:106).

As assessed by TaqMan analysis, 2099 mRNA was expressed at a nine-fold greater level in colon tumors (single sample pools of three tumors each) vs. normal colon tissue pool. HUVEC cells, pituitary gland, brain and erythroid samples showed high expression of 2099 mRNA. Further TaqMan analysis on oncology tissue panels indicated that 2099 mRNA was expressed at greatly increased levels in 6/6 primary lung tumors vs. 3/3 normal lung samples. 2099 mRNA was also expressed at five-fold higher levels in proliferating HMVEC cells vs. arrested HMVECs. TaqMan experiments completed on the expanded lung cancer panel indicated that 2099 mRNA was expressed at an increased level in 7/11 moderately or poorly differentiated non-small cell lung carcinoma samples vs. 4/4 normal lung samples. In the lung cancer cell model panel, 2099 mRNA expression is reduced upon p53 expression and increased upon expression of activated kras. 2099 mRNA expression is reduced 3-fold in H460 cells with transiently expressed wild-type p53 at 24 hrs post infection. In H125 cells expressing a fusion protein of p53 with the hormone binding domain of the estrogen receptor, 4-hydroxytamoxifen induces activity of p53 and also reduced expression of 2099 mRNA. In the K10 clone of activated kras-transformed BEAS 2B cells, 2099 mRNA is expressed at slightly increased levels in comparison to the nontransformed, parental BEAS 2B lung cells. ISH analysis of 2099 mRNA shows similar expression patterns in lung and breast primary carcinomas as in the TaqMan results.

2099 is an effector of Rho-family GTPases (Rho and Rac) and interacts with the adapter protein NCK. NCK binds EGFR, PDGFR, VEGFR and IRS-1 (SH2) and has been shown to induce cellular transformation. Therefore, NCK may couple receptor tyrosine kinase (RTK) activation with Rho family signaling through PKL2. Phosphoinositide-dependent kinase 1 (PDK1) phosphorylates and activates PKL2.

Due to 2099 function and expression, modulators of 2099 activity would mediate RTK growth, survival and/or motility signals and would inhibit tumor progression. Modulators of 2099 activity would be useful in treating human cancers, including but not limited to lung cancer. 2099 polypeptides of the present invention are useful in screening for modulators of 2099 activity.

Gene ID 2150

The human 2150 sequence (SEQ ID NO:107) known also pim-2 protooncogene (PIM2), is approximately 2088 nucleotides long including untranslated regions. The coding sequence, located at about nucleic acids 186 to 1190 of SEQ ID NO:107, encodes a 334 amino acid protein (SEQ ID NO:108).

As assessed by TaqMan analysis, 2150 mRNA was expressed at seven-fold increased levels in the primary colon tumor, primary lung tumor and primary breast tumor pools (single sample pools of three tumors each), vs. the normal colon, lung and breast tissues pools, respectively. Erythroid, tonsil and lymph node samples also showed high expression of 2150 mRNA. Further TaqMan analysis on oncology tissue panels indicated that 2150 mRNA was expressed at increased levels in 6/6 primary lung tumors vs. 2/3 normal lung samples. In TaqMan experiments completed on the colon cancer cell model panel, 2150 mRNA expression is reduced (9-17 fold) in variants of the parental HCT116 cell line that have the activated kras allele knocked out vs. HCT116 cells with an activated kras allele intact. e3HAMRAS9 cells with re-expressed activated kras have a 7-fold increase in 2150 mRNA expression vs. parental HKE3 cells with a knocked out activated kras allele. 2150 mRNA was also expressed in HK26 cells that have an intact kras allele.

Pim-2 expression correlates with activated kras expression in colon cancer cell lines. Experiments have shown that mouse pim-2 collaborates potently with c-myc, even when modestly overexpressed, to cause lymphomas.

Due to 2150 mRNA expression, modulators of 2150 activity would be useful in treating human cancers, including but not limited to lung and colon cancer. 2150 polypeptides of the present invention are useful in screening for modulators of 2150 activity.

Gene ID 26583

The human 26583 sequence (SEQ ID NO:109) known also as pyruvate dehydrogenase phosphate 1 (PDP1), is approximately 2838 nucleotides long including untranslated regions. The coding sequence, located at about nucleic acids 462 to 2075 of SEQ ID NO:109, encodes a 537 amino acid protein (SEQ ID NO:110).

As assessed by TaqMan analysis, 26583 mRNA was expressed at seven-fold increased levels in colon tumor pool (single sample pools of three tumors each) vs. the normal colon tissue pool. Brain, heart and skeletal muscle samples showed high expression of 26583 mRNA. Further TaqMan analysis on oncology tissue panels indicated that 26583 mRNA was expressed at higher levels in 2/5 primary breast tumors vs. 3/3 normal breast tissue samples, 2/6 primary ovarian tumors vs. 3/3 normal ovary tissue samples, 3/6 primary lung tumors vs. 3/3 normal lung tissue samples, and 3/4 primary colon tumors vs. 3/3 normal colon tissue samples. It is expressed at two-fold increased levels in proliferating HMVEC cells vs. arrested HMVECs. TaqMan experiments completed on the ExCCa panel indicated that 26583 mRNA was expressed at an increased level in 6/13 primary colon tumors vs. 4/6 normal colon tissue samples. In addition, 4/5 colon metastasis to liver had increased expression when compared to 6/6 normal liver tissue samples. Further TaqMan experiments on the colon metastases to liver panel indicated that 26583 mRNA was expressed in 11/16 colon metastases to liver vs. 3/3 normal liver tissue samples. In the panel of matched ovarian metastases to primary ovarian tumors, 26583 mRNA is expressed at increased levels in 6/7 pairs. TaqMan experiments on the oncology xenograft cell line panel indicated that 26583 mRNA is expressed in all tumor cell lines, with very high levels of expression in colon cancer cell lines, including DLD1, HCT116 and SW480. In the colon cancer cell model panel, 26583 mRNA expression is reduced in variants of the parental HCT116 cell that have the activated kras allele knocked out. In the breast cancer cell model panel, 26583 mRNA expression increases four-fold in the normal breast epithelial cell line, MCF10A, after 1 hr treatment with 10 ng.ml EGF.

26583 is a mitochondrial enzyme associated with the pyruvate dehydrogenase complex (PDC). It is involved in the regulation of utilization of carbohydrate fuels, and catalyzes dephosphorylation and concomitant reactivation of the E1 component of the PDC. This activity is prominent in tissues with high energy demands, such as brain, skeletal muscle and heart. Acetyl-CoA is formed from pyruvate through oxidative decarboxylation by the multienzyme complex pyruvate dehydrogenase. The pyruvate dehydrogenase complex contains three catalytic activities (E1, E2 and E3). The pyruvate dehydrogenase complex regulates the entrance of acetyl units derived from carbohydrate sources into the citric acid cycle. The decarboxylation of pyruvate by E1 is irreversible and, since there are no other pathways in mammals for the synthesis of acetyl-CoA from pyruvate, it is crucial that the reaction be carefully controlled. Two regulatory systems are employed: 1) Product inhibition by NADH and acetyl Co-A and 2) Covalent modification by phosphorylation/dephosphorylation of the pyruvate dehydrogenase E1 subunit by pyruvate dehydrogenase kinase and pyruvate dehydrogenase phosphatase (26583). These enzymes are bound to the E2 core. The kinase phosphorylates and inactivates E1; the phosphatase (26583) removes this phosphate, thus reactivating the complex.

The pyruvate dehydrogenase complex (PDC) regulates the use of glucose-linked substrates as sources of oxidative energy or as precursors in the biosynthesis of fatty acids. Activity of PDC is regulated by the competing activities of PDK and PDP reactions. Tissue-specific and metabolic state-specific control is achieved by the selective expression and distinct regulatory properties of at least four PDK isozymes and two PDP isozymes.

Elevated PDP1 activity in tumors will increase PDC production of acetyl-CoA and/or fatty acids, supporting increased tumor cell metabolism and proliferation. Due to 26583 mRNA function and expression, modulators of 26583 activity would affect tumor growth. Modulators of 26583 activity would be useful in treating human cancers, including but not limited to colon and ovarian cancer. 26583 polypeptides of the present invention are useful in screening for modulators of 26583 activity.

Gene ID 2784

The human 2784 sequence (SEQ ID NO:111), also known as placental leucine aminopeptidase (P-LAP) or leucyl-cystinyl aminopeptidase, is approximately 4053 nucleotides long including untranslated regions. The coding sequence, located at about nucleic acids 472 to 3549 of SEQ ID NO:111, encodes a 1025 amino acid protein (SEQ ID NO:112).

As assessed by TaqMan analysis, 2784 mRNA showed increased expression in 6/6 lung tumor samples vs. normal lung tissue. 2784 mRNA was expressed at increased levels in the primary colon tumor pool (single sample pool of three tumors each), vs. the normal colon pool. Further TaqMan analysis on oncology tissue panels indicated that 2784 mRNA was expressed at increased levels in 3/5 colon tumors vs. 2/3 normal colon samples. TaqMan experiments also indicated that 2784 mRNA is overexpressed in cells expressing mutant kras. In transcriptional profiling experiments, 2784 mRNA showed increased expression in Beas-2B cells that express mutated kras.

The predicted activity of 2784 is degradation of hormone peptides, particularly oxytocin and vasopressin. Vasopressin is thought to increase proliferation in small cell lung cancer. Increased 2784 expression may be required to maintain optimal peptide hormone homeostasis in lung tumor growth.

Due to 2784 mRNA expression and function, modulators of 2784 activity would be useful in treating human cancers, including but not limited to lung cancer. 2784 polypeptides of the present invention are useful in screening for modulators of 2784 activity.

Gene ID 8941

The human 8941 sequence (SEQ ID NO:113), also known as aldehyde reductase or alcohol dehydrogenase [NADP+], is approximately 1537 nucleotides long including untranslated regions. The coding sequence, located at about nucleic acids 446 to 1423 of SEQ ID NO:113, encodes a 325 amino acid protein (SEQ ID NO:114).

As assessed by TaqMan analysis, 8941 mRNA was expressed at higher levels in colon and lung tumor pools (single sample pool of 3 tumors) vs. normal colon and lung pools (single sample pool of 3 tissue samples). 8941 mRNA was also highly expressed in human umbilical vein endothelial cells (HUVEC). Further TaqMan analysis on oncology tissue panels indicated that 8941 mRNA was expressed at increased levels in 4/6 breast, 6/6 lung, and 2/5 colon tumors when compared to their respective normal tissue samples (3/3 breast, 3/3 lung, 3/3 colon). 8941 mRNA was expressed at increased levels in proliferating human microvascular endothelial cells (HMVEC) vs. arrested HMVECs. TaqMan analysis of the angiogenesis 1 panel (fetal vs. adult tissues) indicated that 8941 mRNA was expressed at increased levels in fetal kidney (2/2) and heart (1/1) vs. adult angiogenic tissue samples (35/35), including adult heart (7/7). TaqMan analysis of the angiogenesis 2 panel (hemangiomas and necrotic and angiogenic tumors) indicated that 8941 mRNA was expressed at higher levels in 6/8 hemangiomas vs. 1/1 normal skin tissue samples. 8941 was also expressed at higher levels in necrotic tumors (1/3 breast, 2/3 colon, and 3/4 lung tumors). In the expanded lung tumor panel, TaqMan analysis indicated that 8941 mRNA was increased in 16/33 lung tumors vs. 6/6 normal lung tissue samples or 27/33 lung tumors vs. 5/6 normal lung tissue samples.

Alkenyl aldehyde reductases are critical components of the oxidative stress response. Tumors substantially suppress the accumulation of lipid peroxidation byproducts to prevent apoptosis. Inhibition of alkenyl aldehyde reductases will result in accumulation of these lipid peroxidation byproducts, resulting in apoptosis in those cells dependent on its upregulated activity, in particular tumor cells.

Due to 8941 mRNA function and expression, modulators of 8941 activity would be useful in treating human cancers, including but not limited to lung cancer and tumor angiogenesis. 8941 polypeptides of the present invention are useful in screening for modulators of 8941 activity.

Gene ID 9811

The human 9811 sequence (SEQ ID NO:115) known also as guanylate cyclase soluble, beta-1 chain (GCS-beta-1 or GUCY1B1), is approximately 2443 nucleotides long including untranslated regions. The coding sequence, located at about nucleic acids 89 to 1948 of SEQ ID NO:115, encodes a 619 amino acid protein (SEQ ID NO:116).

As assessed by TaqMan analysis, 9811 mRNA was highly expressed in central nervous system tissues, megakaryocytes and hemangioma (single sample pools of 3), with lower expression in cardiovascular tissues and substantially lower expression in other tissues, including breast, ovary, colon, and lung tumors. Further TaqMan analysis on oncology tissue panels indicated that 9811 mRNA was expressed at increased levels in 3/6 ovarian tumors and 6/6 lung tumors vs. their respective normal tissues (3/3 ovary and 2/2 lung samples), and was highly expressed in hemangioma. 9811 mRNA was also increased in proliferating HMVEC tissue culture vs. arrested HMVEC tissue culture. TaqMan analysis on angiogenesis I panel (fetal vs. adult tissues), indicated that expression of 9811 was higher in fetal kidney (2/2) and umbilical cord (2/2) vs. angiogenic adult tissues on the panel. TaqMan analysis on angiogenesis II panel (hemangiomas, necrotic and angiogenic tumor tissues) indicated higher expression of 9811 mRNA in 8/8 hemangioma tissues vs. 1/1 normal skin. There was higher expression in 1/4 breast, 3/3 colon, 2/4 ovary, and 3/3 lung tumors vs. their respective normal tissues (1/1 breast, 1/1 colon, 1/1 ovary, and 1/1 lung). In addition, 9811 mRNA was expressed at higher levels in 1/1 Wilm's tumor sample vs. 1/1 normal kidney sample. In the expanded lung tumor panel, TaqMan analysis indicated higher expression of 9811 mRNA in 7/7 non-small cell lung cancer (NSCLC) tumors and 1/1 small cell lung cancer (SCLC) tumors vs. 3/4 normal lung tissue samples. TaqMan experiments completed on in vitro endothelial cell proliferation, arrest, and tube formation (EC/PAT) panels indicated that 9811 mRNA was expressed at very low levels (19/19 samples) except in HMVEC tube formation supported on matrigel (3/3 samples) where it was much more highly expressed. TaqMan experiments completed on hemangiomas indicated that 9811 mRNA was expressed at very low levels in tissue culture (20/20 samples), but at much higher levels in clinical hemangioma tissue samples (3/3).

Tumor cells rely on nitric oxide (NO) signaling through soluble guanylate cyclase to promote proliferation, survival, angiogenesis, and migration. 9811 is a key mediator of NO signaling and shows restricted expression consistent with its involvement in angiogenesis.

Due to 9811 mRNA function and expression, modulators of 9811 activity would affect tumor growth. Modulators of 9811 activity would be useful in treating human cancers, including but not limited to tumor angiogenesis. 9811 polypeptides of the present invention are useful in screening for modulators of 9811 activity.

Gene ID 27444

The human 27444 sequence (SEQ ID NO:117) known also as guanylate cyclase soluble, beta-2 chain (GUCY1B2), is approximately 2749 nucleotides long including untranslated regions. The coding sequence, located at about nucleic acids 281 to 2134 of SEQ ID NO:117, encodes a 617 amino acid protein (SEQ ID NO:118).

As assessed by TaqMan analysis, 27444 mRNA is expressed at a 16-fold increased level in the primary breast tumor pool vs. the normal breast tissue pool and at a 7-fold increased level in the primary lung tumor pool vs. the normal lung tissue pool. In the oncology tissue panel, 27444 mRNA is not expressed in any normal tissue sample and is expressed in only 1/5 primary breast tumors, 2/6 primary ovary tumors, 1/6 primary lung tumors and 2/4 primary colon tumors. In further TaqMan analysis on the expanded lung cancer tissue panel, 27444 mRNA is expressed at increased levels (2-11 fold) in 7/20 primary lung tumor samples vs. 4/4 normal lung tumor samples. In the expanded breast cancer tissue panel, 27444 mRNA is not expressed in any normal tissue samples and is expressed at low levels in 1/4 invasive lobular carcinomas and 4/11 ductal carcinomas. In the expanded colon cancer tissue panel, 27444 mRNA showed no expression in 5/6 normal colon samples with little expression in the remaining sample. 27444 mRNA is expressed, at low levels, in 8/13 primary colon tumors and expressed in 3/3 colon metastases to liver, with higher expression levels in 2/3 liver metastases. Comparatively, 27444 mRNA is not expressed in 5/6 normal liver samples and is expressed at a low level in the remaining normal liver sample. TaqMan analysis on the angiogenesis 1 panel showed 27444 mRNA expression in uterine tumors, placenta and fetal kidney. In the angiogenesis 2 panel, 27444 mRNA showed expression in many highly angiogenic tumor types, including hemangiomas, endometrium tumors and hypoxic breast, colon, ovary and lung tumors. A Wilm's tumor sample showed 9-fold greater expression vs. a normal kidney sample. In the oncology xenograft cell line panel, 27444 mRNA is expressed in a subset of tumor cell lines, with significant expression in NCI-H69, MCF-7 and NCCCI-H322.

Tumor cells rely on nitric oxide signaling through soluble guanylate cyclase to promote proliferation, survival, angiogenesis, and migration. 27444 is a key mediator of nitric oxide signaling and shows restricted expression in a subset of tumors.

Due to 27444 mRNA function and expression, modulators of 27444 activity would modulate tumor growth, vascularization, and metastasis. Modulators of 27444 activity would be useful in treating human cancers, including but not limited to lung cancer. 27444 polypeptides of the present invention are useful in screening for modulators of 27444 activity.

Gene ID 50566

The human 50566 sequence (SEQ ID NO:119) is approximately 1154 nucleotides long including untranslated regions. The coding sequence, located at about nucleic acids 22 to 870 of SEQ ID NO:119, encodes a 282 amino acid protein (SEQ ID NO:120).

As assessed by TaqMan analysis, 50566 mRNA is expressed at a 9-fold increased level in the primary breast tumor pool vs. the normal breast tissue pool, at a 5-fold increased level in the primary ovary tumor pool vs. the normal ovary tissue pool, and at an 11-fold increased level in the primary colon tumor pool vs. the normal colon tissue pool. Brain and erythroid cell samples also showed high expression of 50566 mRNA. Further TaqMan analysis on oncology tissue panels indicated that 50566 mRNA was expressed at increased levels (3-9 fold) in 3/5 primary breast tumor samples vs. 3/3 normal breast tumor samples. 50566 mRNA is expressed at increased levels (3-48 fold) in 4/6 primary ovary tumor samples vs. 2/3 normal ovary tumor samples. 50566 mRNA is also expressed at increased levels (5-159 fold) in 4/4 primary colon tumor samples vs. 3/3 normal colon tumor sample. TaqMan analysis on the expanded breast cancer tissue panel showed 50566 mRNA is expressed at increased levels (2-61 fold) in 10/15 primary breast tumor samples vs. 3/4 normal breast tumor samples. In the expanded colon cancer tissue panel, 50566 mRNA is expressed at increased levels (2-43 fold) in 9/15 primary colon tumor samples vs. 6/6 normal colon tumor samples and is highly expressed in 4/4 colon metastases to the liver with little expression in 6/6 normal liver samples. In the expanded ovary cancer tissue panel, 50566 mRNA is expressed at increased levels (2-44 fold) in 8/17 primary ovary tumor samples vs. 5/5 normal ovary tumor samples. Further TaqMan analysis on the colon cancer cell model panel showed 50566 mRNA expression is reduced in HKH2 and HKE3 cells vs. HCT116 and HK26 cells (wild-type kras vs. activated kras). 50566 mRNA expression is also reduced in DLD1 cells with transiently expressed p53. In the oncology xenograft cell line panel, 50566 mRNA is expressed in all tumor cell lines, with very high expression in MCF-7 cells followed by ZR-75 and NCI-H69 cells. The ISH experiment confirmed the TaqMan data for colon and ovarian tumors.

50566 is an unnamed human protein with 48% identity to hydroxyacylglutathione hydrolase (Glyoxalase II). 50566 may play a role as a detoxifying enzyme in tumors, perhaps in the glyoxalase system, by detoxifying cytotoxic methylglyoxal and modulating S-D-lactoylglutathione levels. Inhibition of 50566 is hypothesized to increase intracellular concentrations of the toxic methylglyoxal and/or the growth inhibitory intermediate S-D-lactoylglutathione, thereby inhibiting tumor growth.

Due to 50566 mRNA expression, modulators of 50566 activity would be useful in treating human cancers, including but not limited to ovarian and colon cancer. 50566 polypeptides of the present invention are useful in screening for modulators of 50566 activity.

Gene ID 66428

The human 66428 sequence (SEQ ID NO:121), a putative aldo-keto reductase, is approximately 1680 nucleotides long including untranslated regions. The coding sequence, located at about nucleic acids 101 to 1024 of SEQ ID NO:121, encodes a 307 amino acid protein (SEQ ID NO:122).

As assessed by TaqMan analysis, 66428 mRNA was expressed at increased levels in 1/1 ovary, 1/1 colon, and 1/1 lung tumor pools (single sample pools of 3 tumors each) vs. their respective normal tissue pools. Further TaqMan analysis on oncology tissue panels indicated that 66428 mRNA was expressed in 3/6 lung and 1/5 colon tumors vs. normal tissue samples (3/3 lung and 3/3 colon). TaqMan analysis on the angiogenesis 1 panel (fetal vs. adult tissues) indicated that 9/12 fetal tissues had higher 66428 mRNA expression vs. 31/35 adult tissues. In particular, 66428 mRNA levels were higher in fetal heart (1/1) relative to adult heart (7/7). TaqMan analysis on the angiogenesis 2 panel (hemangiomas and necrotic and angiogenic tumors) indicated that 66428 mRNA was elevated in hemangiomas (5/8) vs. normal skin tissue (1/1) and in necrotic tumors (3/4 breast, 3/3 colon, 2/4 ovary, and 2/3 lung) vs. normal tissues (1/1 breast, 1/1 colon, 1/1 ovary, and 1/1 lung) and in Wilm's tumor (1/1) vs. normal kidney (1/1). On the TaqMan panel evaluating in vitro endothelial cell proliferation, arrest and tube formation (EC/PAT), 66428 mRNA was expressed at increased levels in proliferating lung HMVEC (1/1) and HUVEC (2/2) samples vs. arrested lung HMVEC (1/1) and HUVEC (2/2) samples. Also, 66428 mRNA was elevated in lung HMVEC (5/6) and HUVEC (3/3) tube formation samples on matrigel vs. arrested lung HMVEC (1/1) and HUVEC (1/1) samples. TaqMan analysis of colon adenocarcinoma panel indicated that 66428 mRNA was increased in 7/13 colon adenocarcinomas vs. 5/6 normal colon tissue samples. In addition, 66428 mRNA was increased in 4/4 colon metastases to the liver vs. both normal liver (5/5) and normal colon (6/6). On the colon metastasis panel, TaqMan analysis indicated that 66428 mRNA was increased in 4/4 primary colon tumors vs. 3/3 normal colon tissue. In addition, 66428 mRNA was higher in 10/15 colon metastases vs. normal colon (3/3), normal liver (2/2) or primary colon tumors (4/4). In addition, 66428 mRNA levels were increased in 12/14 other sites of metastasis vs. normal colon (3/3) or primary colon tumors (4/4). The ISH experiment showed 66428 mRNA expression in primary colon tumors and metastatic colon carcinoma to the liver. In addition, selected angiogenic tissues were also positive.

Alkenyl aldehyde reductases are critical components of oxidative stress response. Tumors substantially suppress the accumulation of lipid peroxidation byproducts to prevent apoptosis. Inhibition of alkenyl aldehyde reductases will result in accumulation of these lipid peroxidation byproducts, resulting in apoptosis in those cells dependent on its upregulated activity, in particular tumor cells.

Due to 66428 mRNA expression, modulators of 66428 activity would be useful in treating human cancers, including but not limited to colon cancer. 66428 polypeptides of the present invention are useful in screening for modulators of 66428 activity.

Various aspects of the invention are described in further detail in the following subsections:

Screening Assays:

The invention provides a method (also referred to herein as a “screening assay”) for identifying modulators, i.e., candidate or test compounds or agents (e.g., peptides, peptidomimetics, small molecules (organic or inorganic) or other drugs) which bind to 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 proteins, have a stimulatory or inhibitory effect on, for example, 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 expression or 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 activity, or have a stimulatory or inhibitory effect on, for example, the expression or activity of a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 substrate. Compounds identified using the assays described herein may be useful for treating a cancer.

These assays are designed to identify compounds that bind to a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein, bind to other intracellular or extracellular proteins that interact with a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein, and interfere with the interaction of the 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein with other intercellular or extracellular proteins. For example, in the case of the 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein, which is a transmembrane receptor-type protein, such techniques can identify ligands for such a receptor. A 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein ligand or substrate can, for example, be used to ameliorate at least one symptom of a cancer. Such compounds may include, but are not limited small molecules, peptides, antibodies, ribozymes, gene therapy vectors and antisense oligonucleotides. Such compounds may also include other cellular proteins.

Compounds identified via assays such as those described herein may be useful, for example, for treating a cancer. In instances whereby a cancer condition results from an overall lower level of 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 gene expression and/or 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein in a cell or tissue, compounds that interact with the 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein may include compounds which accentuate or amplify the activity of the bound 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236,2099,2150, 26583, 2784, 8941, 9811, 27444, 50566 or 66428 protein. Such compounds would bring about an effective increase in the level of 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein activity, thus ameliorating symptoms.

In other instances, mutations within the 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 gene may cause aberrant types or excessive amounts of 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 proteins to be made which have a deleterious effect that leads to a cancer. Similarly, physiological conditions may cause an excessive increase in 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 gene expression leading to a cancer. In such cases, compounds that bind to a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein may be identified that inhibit the activity of the 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642,;85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein. Assays for testing the effectiveness of compounds identified by techniques such as those described in this section are discussed herein.

In one embodiment, the invention provides assays for screening candidate or test compounds which are substrates of a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein or polypeptide or biologically active portion thereof. In another embodiment, the invention provides assays for screening candidate or test compounds which bind to or modulate the activity of a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein or polypeptide or biologically active portion thereof. The test compounds of the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner U.S. Pat. No. '409), plasmids (Cull et al. (1992) Proc Natl Acad Sci USA 89:1865-1869) or on phage (Scott and Smith (1990) Science 249:386-390); (Devlin (1990) Science 249:404-406); (Cwirla et al. (1990) Proc. Natl. Acad. Sci. 87:6378-6382); (Felici (1991) J. Mol. Biol. 222:301-310); (Ladner supra.).

In one embodiment, an assay is a cell-based assay in which a cell which expresses a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein or biologically active portion thereof is contacted with a test compound and the ability of the test compound to modulate 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 activity is determined. Determining the ability of the test compound to modulate 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784,8941, 9811, 27444, 50566 or 66428 activity can be accomplished by monitoring, for example, intracellular calcium, IP₃, cAMP, or diacylglycerol concentration, the phosphorylation profile of intracellular proteins, cell proliferation and/or migration, gene expression of, for example, cell surface adhesion molecules or genes associated with cancer, or the activity of a 140, 1470, 1686, 2089, 2427, 3702,5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893,33230, 1586, 9943, 16334, 68862, 9011, 14031, 6178, 21225, 1420, 32236, 2099, 2150, 26583, 2784, 8941, 9811, 27444, 50566 or 66428-regulated transcription factor. The cell can be of mammalian origin, e.g., a cancer cell. In one embodiment, compounds that interact with a receptor domain can be screened for their ability to function as ligands, i.e., to bind to the receptor and modulate a signal transduction pathway. Identification of ligands, and measuring the activity of the ligand-receptor complex, leads to the identification of modulators (e.g., antagonists) of this interaction. Such modulators may be useful in the treatment of a cancer.

The ability of the test compound to modulate 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 binding to a substrate or to bind to 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 can also be determined. Determining the ability of the test compound to modulate 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990,-2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 binding to a substrate can be accomplished, for example, by coupling the 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 substrate with a radioisotope or enzymatic label such that binding of the 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 substrate to 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 can be determined by detecting the labeled 140, 1470, 1686, 2089, 2427, 3702,5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 substrate in a complex. 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 could also be coupled with a radioisotope or enzymatic label to monitor the ability of a test compound to modulate 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 binding to a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 substrate in a complex. Determining the ability of the test compound to bind 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 can be accomplished, for example, by coupling the compound with a radioisotope or enzymatic label such that binding of the compound to 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893,33230, 1586, 9943, 16334, 68862, 9011, 14031, 6178, 21225, 1420, 32236, 2099, 2150, 26583, 2784, 8941, 9811, 27444, 50566 or 66428 can be determined by detecting the labeled 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 compound in a complex. For example, compounds (e.g., 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088,32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 ligands or substrates) can be labeled with ¹²⁵I, ³⁵S, ¹⁴C, or ³H, either directly or indirectly, and the radioisotope detected by direct counting of radioemmission or by scintillation counting. Compounds can further be enzymatically labeled with, for example, horseradish peroxidase, alkaline phosphatase, or luciferase, and the enzymatic label detected by determination of conversion of an appropriate substrate to product.

It is also within the scope of this invention to determine the ability of a compound (e.g., a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 ligand or substrate) to interact with 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 without the labeling of any of the interactants. For example, a microphysiometer can be used to detect the interaction of a compound with 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 without the labeling of either the compound or the 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 (McConnell, H. M. et al. (1992) Science 257:1906-1912. As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between a compound and 140, 1470, 1686, 2089, 2427, 3702,5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893,33230, 1586, 9943, 16334, 68862, 9011, 14031, 6178, 21225, 1420, 32236, 2099, 2150, 26583, 2784, 8941, 9811, 27444, 50566 or 66428.

In another embodiment, an assay is a cell-based assay comprising contacting a cell expressing a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 target molecule (e.g., a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 substrate) with a test compound and determining the ability of the test compound to modulate (e.g., stimulate or inhibit) the activity of the 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 target molecule. Determining the ability of the test compound to modulate the activity of a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 target molecule can be accomplished, for example, by determining the ability of the 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein to bind to or interact with the 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 target molecule.

Determining the ability of the 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784,8941, 9811, 27444, 50566 or 66428 protein or a biologically active fragment thereof, to bind to or interact with a 140, 1470, 1686, 2089, 2427, 3702,5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 target molecule can be accomplished by one of the methods described above for determining direct binding. In a preferred embodiment, determining the ability of the 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein to bind to or interact with a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 target molecule can be accomplished by determining the activity of the target molecule. For example, the activity of the target molecule can be determined by detecting induction of a cellular second messenger of the target (i.e., intracellular Ca²+, diacylglycerol, IP₃, cAMP), detecting catalytic/enzymatic activity of the target on an appropriate substrate, detecting the induction of a reporter gene (comprising a target-responsive regulatory element operatively linked to a nucleic acid encoding a detectable marker, e.g., luciferase), or detecting a target-regulated cellular response (e.g., gene expression).

In yet another embodiment, an assay of the present invention is a cell-free assay in which a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein or biologically active portion thereof, is contacted with a test compound and the ability of the test compound to bind to the 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein or biologically active portion thereof is determined. Preferred biologically active portions of the 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 proteins to be used in assays of the present invention include fragments which participate in interactions with non-140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124,4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 molecules, e.g., fragments with high surface probability scores. Binding of the test compound to the 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444,50566 or 66428 protein can be determined either directly or indirectly as described above. In a preferred embodiment, the assay includes contacting the 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein or biologically active portion thereof with a known compound which binds 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 to form an assay mixture, contacting the assay mixture with a test compound, and determining the ability of the test compound to interact with a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein, wherein determining the ability of the test compound to interact with a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein comprises determining the ability of the test compound to preferentially bind to 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 or biologically active portion thereof as compared to the known compound. Compounds that modulate the interaction of 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444,50566 or 66428 with a known target protein may be useful in regulating the activity of a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein, especially a mutant 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein.

In another embodiment, the assay is a cell-free assay in which a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein or biologically active portion thereof is contacted with a test compound and the ability of the test compound to modulate (e.g., stimulate or inhibit) the activity of the 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein or biologically active portion thereof is determined. Determining the ability of the test compound to modulate the activity of a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784,8941, 9811, 27444, 50566 or 66428 protein can be accomplished, for example, by determining the ability of the 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893,33230, 1586, 9943, 16334, 68862, 9011, 14031, 6178, 21225, 1420, 32236, 2099, 2150, 26583, 2784, 8941, 9811, 27444, 50566 or 66428 protein to bind to a 140, 1470, 1686, 2089, 2427, 3702,5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 target molecule by one of the methods described above for determining direct binding. Determining the ability of the 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein to bind to a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 target molecule can also be accomplished using a technology such as real-time Biomolecular Interaction Analysis (BIA) (Sjolander, S. and Urbaniczky, C. (1991) Anal. Chem. 63:2338-2345 and Szabo et al. (1995) Curr. Opin. Struct. Biol. 5:699-705). As used herein, “BIA” is a technology for studying biospecific interactions in real time, without labeling any of the interactants (e.g., BIAcore). Changes in the optical phenomenon of surface plasmon resonance (SPR) can be used as an indication of real-time reactions between biological molecules.

In another embodiment, determining the ability of the test compound to modulate the activity of a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein can be accomplished by determining the ability of the 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein to further modulate the activity of a downstream effector of a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 target molecule. For example, the activity of the effector molecule on an appropriate target can be determined or the binding of the effector to an appropriate target can be determined as previously described.

In yet another embodiment, the cell-free assay involves contacting a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein or biologically active portion thereof with a known compound which binds the 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein to form an assay mixture, contacting the assay mixture with a test compound, and determining the ability of the test compound to interact with the 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein, wherein determining the ability of the test compound to interact with the 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein comprises determining the ability of the 140, 1470, 1686, 2089, 2427, 3702, 5891, 6428, 7181,7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein to preferentially bind to or modulate the activity of a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 target molecule.

In more than one embodiment of the above assay methods of the present invention, it may be desirable to immobilize either 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 or its target molecule to facilitate separation of complexed from uncomplexed forms of one or both of the proteins, as well as to accommodate automation of the assay. Binding of a test compound to a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein, or interaction of a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein with a target molecule in the presence and absence of a candidate compound, can be accomplished in any vessel suitable for containing the reactants. Examples of such vessels include microtitre plates, test tubes, and micro-centrifuge tubes. In one embodiment, a fusion protein can be provided which adds a domain that allows one or both of the proteins to be bound to a matrix. For example, glutathione-S-transferase/140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 fusion proteins or glutathione-S-transferase/target fusion proteins can be adsorbed onto glutathione sepharose beads (Sigma Chemical, St. Louis, Mo.) or glutathione derivatized microtitre plates, which are then combined with the test compound or the test compound and either the non-adsorbed target protein or 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein, and the mixture incubated under conditions conducive to complex formation (e.g., at physiological conditions for salt and pH). Following incubation, the beads or microtitre plate wells are washed to remove any unbound components, the matrix immobilized in the case of beads, complex determined either directly or indirectly, for example, as described above. Alternatively, the complexes can be dissociated from the matrix, and the level of 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784,8941, 9811, 27444, 50566 or 66428 binding or activity determined using standard techniques.

Other techniques for immobilizing proteins on matrices can also be used in the screening assays of the invention. For example, either a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein or a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 target molecule can be immobilized utilizing conjugation of biotin and streptavidin. Biotinylated 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein or target molecules can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical). Alternatively, antibodies reactive with 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein or target molecules but which do not interfere with binding of the 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein to its target molecule can be derivatized to the wells of the plate, and unbound target or 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein trapped in the wells by antibody conjugation. Methods for detecting such complexes, in addition to those described above for the GST-immobilized complexes, include immunodetection of complexes using antibodies reactive with the 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein or target molecule, as well as enzyme-linked assays which rely on detecting an enzymatic activity associated with the 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein or target molecule.

In another embodiment, modulators of 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 expression are identified in a method wherein a cell is contacted with a candidate compound and the expression of 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 98,11, 27444, 50566 or 66428 mRNA or protein in the cell is determined. The level of expression of 140, 1470, 1686, 2089, 2427, 3702,5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 mRNA or protein in the presence of the candidate compound is compared to the level of expression of 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 mRNA or protein in the absence of the candidate compound. The candidate compound can then be identified as a modulator of 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 expression based on this comparison. For example, when expression of 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178,21225, 1420, 32236, 2099, 2150, 26583, 2784, 8941, 9811, 27444, 50566 or 66428 mRNA or protein is greater (statistically significantly greater) in the presence of the candidate compound than in its absence, the candidate compound is identified as a stimulator of 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 mRNA or protein expression. Alternatively, when expression of 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 mRNA or protein is less (statistically significantly less) in the presence of the candidate compound than in its absence, the candidate compound is identified as an inhibitor of 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 mRNA or protein expression. The level of 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 mRNA or protein expression in the cells can be determined by methods described herein for detecting 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 mRNA or protein.

In yet another aspect of the invention, the 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784,8941, 9811, 27444, 50566 or 66428 proteins can be used as “bait proteins” in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO94/10300), to identify other proteins, which bind to or interact with 140, 1470, 1686, 2089, 2427, 3702,5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 (“140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428-binding proteins” or “140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428-bp”) and are involved in 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 activity. Such 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428-binding proteins are also likely to be involved in the propagation of signals by the 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 proteins or 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 targets as, for example, downstream elements of a 140, 1470, 1686, 2089, 2427, 3702, 5891, 6428,7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428-mediated signaling pathway. Alternatively, such 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428-binding proteins are likely to be 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 inhibitors.

The two-hybrid system is based on the modular nature of most transcription factors, which consist of separable DNA-binding and activation domains. Briefly, the assay utilizes two different DNA constructs. In one construct, the gene that codes for a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428-dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to obtain the cloned gene which encodes the protein which interacts with the 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein.

In another aspect, the invention pertains to a combination of two or more of the assays described herein. For example, a modulating agent can be identified using a cell-based or a cell free assay, and the ability of the agent to modulate the activity of a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein can be confirmed in vivo, e.g., in an animal such as an animal model for a cancer, as described herein.

This invention further pertains to novel agents identified by the above-described screening assays. Accordingly, it is within the scope of this invention to further use an agent identified as described herein in an appropriate animal model. For example, an agent identified as described herein (e.g., a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 modulating agent, an antisense 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 nucleic acid molecule, a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428-specific antibody, or a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428-binding partner) can be used in an animal model to determine the efficacy, toxicity, or side effects of treatment with such an agent. Alternatively, an agent identified as described herein can be used in an animal model to determine the mechanism of action of such an agent. Furthermore, this invention pertains to uses of novel agents identified by the above-described screening assays for treatments as described herein.

Any of the compounds, including but not limited to compounds such as those identified in the foregoing assay systems, may be tested for the ability to ameliorate at least one symptom of a cancer. Cell-based and animal model-based assays for the identification of compounds exhibiting such an ability to ameliorate at least one symptom of a cancer are described herein.

In addition, animal-based models of a cancer, such as those described herein, may be used to identify compounds capable of treating a cancer. Such animal models may be used as test substrates for the identification of drugs, pharmaceuticals, therapies, and interventions which may be effective in treating a cancer. For example, animal models may be exposed to a compound, suspected of exhibiting an ability to treat a cancer, at a sufficient concentration and for a time sufficient to elicit such an amelioration of at least one symptom of a cancer in the exposed animals. The response of the animals to the exposure may be monitored by assessing the reversal of the symptoms of a cancer before and after treatment.

With regard to intervention, any treatments which reverse any aspect of acancer (i.e. have an effect on a cancer including but not limited to cancers of the lung, ovary, prostate, breast, colon or other disease state characterized by modulation of angiogenesis) should be considered as candidates for a human cancer therapeutic intervention. Dosages of test agents may be determined by deriving dose-response curves.

Additionally, gene expression patterns may be utilized to assess the ability of a compound to ameliorate at least one symptom of a cancer. For example, the expression pattern of one or more genes may form part of a “gene expression profile” or “transcriptional profile” which may be then be used in such an assessment. “Gene expression profile” or “transcriptional profile”, as used herein, includes the pattern of mRNA expression obtained for a given tissue or cell type under a given set of conditions. Gene expression profiles may be generated, for example, by utilizing a differential display procedure, Northern analysis and/or RT-PCR. In one embodiment, 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 gene sequences may be used as probes and/or PCR primers for the generation and corroboration of such gene expression profiles.

Gene expression profiles may be characterized for known states, either cancer or normal, within the cell- and/or animal-based model systems. Subsequently, these known gene expression profiles may be compared to ascertain the effect a test compound has to modify such gene expression profiles, and to cause the profile to more closely resemble that of a more desirable profile.

For example, administration of a compound may cause the gene expression profile of a cancer disease model system to more closely resemble the control system. Administration of a compound may, alternatively, cause the gene expression profile of a control system to begin to mimic a cancer or a cancer disease state. Such a compound may, for example, be used in further characterizing the compound of interest, or may be used in the generation of additional animal models.

Cell- and Animal-Based Model Systems

Described herein are cell- and animal-based systems which act as models for cancer. These systems may be used in a variety of applications. For example, the cell- and animal-based model systems may be used to further characterize differentially expressed genes associated with a cancer, e.g., 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428. In addition, animal- and cell-based assays may be used as part of screening strategies designed to identify compounds which are capable of ameliorating at least one symptom of a cancer, as described, below. Thus, the animal- and cell-based models may be used to identify drugs, pharmaceuticals, therapies and interventions which may be effective in treating a cancer. Furthermore, such animal models may be used to determine the LD50 and the ED50 in animal subjects, and such data can be used to determine the in vivo efficacy of potential cancer treatments.

Animal-Based Systems

Animal-based model systems of cancer may include, but are not limited to, non-recombinant and engineered transgenic animals.

Non-recombinant animal models for cancer may include, for example, genetic models.

Models for studying angiogenesis in vivo include tumor cell-induced angiogenesis and tumor metastasis (Hoffman, R M (1998-99) Cancer Metastasis Rev. 17:271-277; Holash, J et al. (1999) Oncogene 18:5356-5362; Li, C Y et al. (2000) J. Natl Cancer Inst. 92:143-147), matrix induced angiogenesis (U.S. Pat. No.5,382,514), the disc angiogenesis system (Kowalski, J. et al. (1992) Exp. Mol. Pathol. 56:1-19), the rodent mesenteric-window angiogenesis assay (Norrby, K (1992) EXS 61:282-286), experimental choroidal neovascularization in the rat (Shen, W Y et al. (1998) Br. J. Ophthalmol. 82:1063-1071), and the chick embryo development (Brooks, P C et al. Methods Mol. Biol. (1999) 129:257-269) and chick embryo chorioallantoic membrane (CAM) models (McNatt L G et al. (1999) J. Ocul. Pharmacol. Ther. 15:413-423; Ribatti, D et al. (1996) Int. J. Dev. Biol. 40:1189-1197), and are reviewed in Ribatti, D and Vacca, A (1999) Int. J. Biol. Markers 14:207-213.

Animal based models for studying tumorigenesis in vivo are well known in the art (reviewed in Animal Models of Cancer Predisposition Syndromes, Hiai, H and Hino, O (eds.) 1999, Progress in Experimental Tumor Research, Vol. 35; Clarke A R Carcinogenesis (2000) 21:435-41) and include, for example, carcinogen-induced tumors (Rithidech, K et al. Mutat Res (1999) 428:33-39; Miller, M L et al. Environ Mol Mutagen (2000) 35:319-327), injection and/or transplantation of tumor cells into an animal, as well as animals bearing mutations in growth regulatory genes, for example, oncogenes (e.g., ras) (Arbeit, J M et al. Am J Pathol (1993) 142:1187-1197; Sinn, E et al. Cell (1987) 49:465-475; Thorgeirsson, S S et al. Toxicol Lett (2000) 112-113:553-555) and tumor suppressor genes (e.g., p53) (Vooijs, M et al. Oncogene (1999) 18:5293-5303; Clark A R Cancer Metast Rev (1995) 14:125-148; Kumar, T R et al. J Intern Med (1995) 238:233-238; Donehower, L A et al. (1992) Nature 356215-221). Furthermore, experimental model systems are available for the study of, for example, ovarian cancer (Hamilton, T C et al. Semin Oncol (1984) 11:285-298; Rahman, N A et al. Mol Cell Endocrinol (1998) 145:167-174; Beamer, W G et al. Toxicol Pathol (1998) 26:704-710), gastric cancer (Thompson, J et al. Int J Cancer (2000) 86:863-869; Fodde, R et al. Cytogenet Cell Genet (1999) 86:105-111), breast cancer (Li, M et al. Oncogene (2000) 19:1010-1019; Green, J E et al. Oncogene (2000) 19:1020-1027), melanoma (Satyamoorthy, K et al. Cancer Metast Rev (1999) 18:401-405), and prostate cancer (Shirai, T et al. Mutat Res (2000) 462:219-226; Bostwick, D G et al. Prostate (2000) 43:286-294).

Additionally, animal models exhibiting a cancer may be engineered by using, for example, 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 gene sequences described above, in conjunction with techniques for producing transgenic animals that are well known to those of skill in the art. For example, 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 gene sequences may be introduced into, and overexpressed in, the genome of the animal of interest, or, if endogenous 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 gene sequences are present, they may either be overexpressed or, alternatively, be disrupted in order to underexpress or inactivate 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 gene expression.

The host cells of the invention can also be used to produce non-human transgenic animals. For example, in one embodiment, a host cell of the invention is a fertilized oocyte or an embryonic stem cell into which 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088,32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428-coding sequences have been introduced. Such host cells can then be used to create non-human transgenic animals in which exogenous 140, 1470, 1686, 2089, 2427, 3702, 5891, 6428, 7181,7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 sequences have been introduced into their genome or homologous recombinant animals in which endogenous 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 sequences have been altered. Such animals are useful for studying the function and/or activity of a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 and for identifying and/or evaluating modulators of 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, and the like. A transgene is exogenous DNA which is integrated into the genome of a cell from which a transgenic animal develops and which remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 gene has been altered by homologous recombination between the endogenous gene and an exogenous DNA molecule introduced into a cell of the animal, e.g., an embryonic cell of the animal, prior to development of the animal.

A transgenic animal used in the methods of the invention can be created by introducing a 140, 1470, 1686, 2089, 2427,.3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428-encoding nucleic acid into the male pronuclei of a fertilized oocyte, e.g., by microinjection, retroviral infection, and allowing the oocyte to develop in a pseudopregnant female foster animal. The 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 cDNA sequence can be introduced as a transgene into the genome of a non-human animal. Alternatively, a nonhuman homologue of a human 140, 1470, 1686, 2089, 2427, 3702,5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 gene, such as a mouse or rat 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 gene, can be used as a transgene. Alternatively, a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 gene homologue, such as another 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 family member, can be isolated based on hybridization to the 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 cDNA sequences and used as a transgene. Intronic sequences and polyadenylation signals can also be included in the transgene to increase the efficiency of expression of the transgene. A tissue-specific regulatory sequence(s) can be operably linked to a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 transgene to direct expression of a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein to particular cells. Methods for generating transgenic animals via embryo manipulation and microinjection, particularly animals such as mice, have become conventional in the art and are described, for example, in U.S. Pat. Nos. 4,736,866 and 4,870,009, both by Leder et al., U.S. Pat. No. 4,873,191 by Wagner et al. and in Hogan, B., Manipulating the Mouse Embryo, (Cold Spring Harbor Laboratory Press, Cold Spring Harbor, N.Y., 1986). Similar methods are used for production of other transgenic animals. A transgenic founder animal can be identified based upon the presence of a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 transgene in its genome and/or expression of 140, 1470, 1686, 2089, 2427, 3702, 5891, 6428, 7181, 7660, 25641, 69583,49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 mRNA in tissues or cells of the animals. A transgenic founder animal can then be used to breed additional animals carrying the transgene. Moreover, transgenic animals carrying a transgene encoding a 140, 1470, 1686, 2089, 2427, 3702, 5891, 6428, 7181, 7660, 25641, 69583, 49863, 8897, 1682, 17667, 9235,3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein can further be bred to other transgenic animals carrying other transgenes.

To create a homologous recombinant animal, a vector is prepared which contains at least a portion of a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 gene into which a deletion, addition or substitution has been introduced to thereby alter, e.g., functionally disrupt, the 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 gene. The 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 gene can be a human gene but more preferably, is a non-human homologue of a human 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 gene. For example, a rat 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 gene can be used to construct a homologous recombination nucleic acid molecule, e.g., a vector, suitable for altering an endogenous 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 gene in the mouse genome. In a preferred embodiment, the homologous recombination nucleic acid molecule is designed such that, upon homologous recombination, the endogenous 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 gene is functionally disrupted (i.e., no longer encodes a functional protein; also referred to as a “knock out” vector). Alternatively, the homologous recombination nucleic acid molecule can be designed such that, upon homologous recombination, the endogenous 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 gene is mutated or otherwise altered but still encodes functional protein (e.g., the upstream regulatory region can be altered to thereby alter the expression of the endogenous 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein). In the homologous recombination nucleic acid molecule, the altered portion of the 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 gene is flanked at its 5′ and 3′ ends by additional nucleic acid sequence of the 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 gene to allow for homologous recombination to occur between the exogenous 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 gene carried by the homologous recombination nucleic acid molecule and an endogenous 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 gene in a cell, e.g., an embryonic stem cell. The additional flanking 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 nucleic acid sequence is of sufficient length for successful homologous recombination with the endogenous gene. Typically, several kilobases of flanking DNA (both at the 5′ and 3′ ends) are included in the homologous recombination nucleic acid molecule (see, e.g., Thomas, K. R. and Capecchi, M. R. (1987) Cell 51:503 for a description of homologous recombination vectors). The homologous recombination nucleic acid molecule is introduced into a cell, e.g., an embryonic stem cell line (e.g., by electroporation) and cells in which the introduced 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 gene has homologously recombined with the endogenous 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 gene are selected (see e.g., Li, E. et al. (1992) Cell 69:915). The selected cells can then injected into a blastocyst of an animal (e.g., a mouse) to form aggregation chimeras (see e.g., Bradley, A. in Teratocarcinomas and Embryonic Stem Cells: A Practical Approach, E. J. Robertson, ed. (IRL, Oxford, 1987) pp. 113-152). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination nucleic acid molecules, e.g., vectors, or homologous recombinant animals are described further in Bradley, A. (1991) Current Opinion in Biotechnology 2:823-829 and in PCT International Publication Nos.: WO 90/11354 by Le Mouellec et al.; WO 91/01140 by Smithies et al.; WO 92/0968 by Zijlstra et al.; and WO 93/04169 by Berns et al.

In another embodiment, transgenic non-human animals for use in the methods of the invention can be produced which contain selected systems which allow for regulated expression of the transgene. One example of such a system is the cre/loxP recombinase system of bacteriophage P1. For a description of the cre/loxP recombinase system, see, e.g., Lakso et al. (1992) Proc. Natl. Acad. Sci. USA 89:6232-6236. Another example of a recombinase system is the FLP recombinase system of Saccharomyces cerevisiae (O'Gorman et al. (1991) Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, I. et al. (1997) Nature 385:810-813 and PCT International Publication Nos. WO 97/07668 and WO 97/07669. In brief, a cell, e.g., a somatic cell, from the transgenic animal can be isolated and induced to exit the growth cycle and enter Gₒ phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell, e.g., the somatic cell, is isolated.

The 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 transgenic animals that express 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 mRNA or a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 peptide (detected immunocytochemically, using antibodies directed against 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 epitopes) at easily detectable levels should then be further evaluated to identify those animals which display a characteristic cancer.

Cell-Based Systems

Cells that contain and express 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 gene sequences which encode a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein, and, further, exhibit cellular phenotypes associated with a cancer, may be used to identify compounds that exhibit an effect on a cancer. Such cells may include non-recombinant monocyte cell lines, such as U937 (ATCC# CRL-1593), THP-1 (ATCC#TIB-202), and P388D1 (ATCC# TIB-63); endothelial cells such as human umbilical vein endothelial cells (HUVECs), human microvascular endothelial cells (HMVEC), and bovine aortic endothelial cells (BAECs); as well as generic mammalian cell lines such as HeLa cells and COS cells, e.g., COS-7 (ATCC# CRL-1651), lung, colon, breast, prostate or ovarian cancer cell lines. Further, such cells may include recombinant, transgenic cell lines. For example, the cancer animal models of the invention, discussed above, may be used to generate cell lines, containing one or more cell types involved in cancer, that can be used as cell culture models for this disorder. While primary cultures derived from the cancer model transgenic animals of the invention may be utilized, the generation of continuous cell lines is preferred. For examples of techniques which may be used to derive a continuous cell line from the transgenic animals, see Small et al., (1985) Mol. Cell Biol. 5:642-648.

Alternatively, cells of a cell type known to be involved in cancer may be transfected with sequences capable of increasing or decreasing the amount of 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 gene expression within the cell. For example, 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 gene sequences may be introduced into, and overexpressed in, the genome of the cell of interest, or, if endogenous 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 gene sequences are present, they may be either overexpressed or, alternatively disrupted in order to underexpress or inactivate 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 gene expression.

In order to overexpress a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 gene, the coding portion of the 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 gene may be ligated to a regulatory sequence which is capable of driving gene expression in the cell type of interest, e.g., an endothelial cell. Such regulatory regions will be well known to those of skill in the art, and may be utilized in the absence of undue experimentation. Recombinant methods for expressing target genes are described above.

For underexpression of an endogenous 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 gene sequence, such a sequence may be isolated and engineered such that when reintroduced into the genome of the cell type of interest, the endogenous 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 alleles will be inactivated. Preferably, the engineered 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 sequence is introduced via gene targeting such that the endogenous 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 sequence is disrupted upon integration of the engineered 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252,9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 sequence into the cell's genome. Transfection of host cells with 140, 1470, 1686, 2089, 2427, 3702, 5891, 6428, 7181, 7660, 2:5641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 genes is discussed, above.

Cells treated with compounds or transfected with 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784,8941, 9811, 27444, 50566 or 66428 genes can be examined for phenotypes associated with cancer.

Transfection of 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 nucleic acid may be accomplished by using standard techniques (described in, for example, Ausubel (1989) supra). Transfected cells should be evaluated for the presence of the recombinant 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 gene sequences, for expression and accumulation of 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 mRNA, and for the presence of recombinant 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein production. In instances wherein a decrease in 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 gene expression is desired, standard techniques may be used to demonstrate whether a decrease in endogenous 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 gene expression and/or in 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893,33230, 1586, 9943, 16334, 68862, 9011, 14031, 6178, 21225, 1420, 32236, 2099, 2150, 26583, 2784, 8941, 9811, 27444, 50566 or 66428 protein production is achieved.

Cellular models for the study of angiogenesis include models of endothelial cell differentiation on Matrigel (Baatout, S. et al. (1996) Rom. J. Intern. Med. 34:263-269; Benelli, R et al. (1999) Int. J. Biol. Markers 14:243-246), embryonic stem cell models of vascular morphogenesis (Doetschman, T. et al. (1993) Hypertension 22:618-629), the culture of microvessel fragments in physiological gels (Hoying, J B et al. (1996) In Vitro Cell Dev. Biol. Anim. 32: 409-419; U.S. Pat. No.5,976,782), and the treatment of endothelial cells and smooth muscle cells with atherogenic and angiogenic factors including growth factors and cytokines (e.g., IL-1β, PDGF, TNFα, VEGF), homocysteine, and LDL. In vitro angiogenesis models are described in, for example, Black, A F et al. (1999) Cell Biol. Toxicol. 15:81-90.

Cellular models for the study of tumorigenesis are known in the art, and include cell lines derived from clinical tumors, cells exposed to chemotherapeutic agents, cells exposed to carcinogenic agents, and cell lines with genetic alterations in growth regulatory genes, for example, oncogenes (e.g., ras) and tumor suppressor genes (e.g., p 53).

Predictive Medicine:

The present invention also pertains to the field of predictive medicine in which diagnostic assays, prognostic assays, and monitoring clinical trials are used for prognostic (predictive) purposes to thereby treat an individual prophylactically. Accordingly, one aspect of the present invention relates to diagnostic assays for determining 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein and/or nucleic acid expression as well as 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 activity, in the context of a biological sample (e.g., blood, serum, cells, e.g., endothelial cells, or tissue, e.g., vascular tissue, bladder tissue or prostate tissue) to thereby determine whether an individual is afflicted with a predisposition or is experiencing a cancer. The invention also provides for prognostic (or predictive) assays for determining whether an individual is at risk of developing a cancer. For example, mutations in a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 gene can be assayed for in a biological sample. Such assays can be used for prognostic or predictive purpose to thereby phophylactically treat an individual prior to the onset of a cancer.

Another aspect of the invention pertains to monitoring the influence of 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 modulators (e.g., anti-140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 antibodies or 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 ribozymes) on the expression or activity of 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 in clinical trials.

These and other agents are described in further detail in the following sections.

Diagnostic Assays

To determine whether a subject is afflicted with a disease, a biological sample may be obtained from a subject and the biological sample may be contacted with a compound or an agent capable of detecting a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein or nucleic acid (e.g., mRNA or genomic DNA) that encodes a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein, in the biological sample. A preferred agent for detecting 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 mRNA or genomic DNA is a labeled nucleic acid probe capable of hybridizing to 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 mRNA or genomic DNA. The nucleic acid probe can be, for example, the 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 nucleic acid set forth in SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119 or 121, or a portion thereof, such as an oligonucleotide of at least 15, 20, 25, 30, 25, 40, 45, 50, 100, 250 or 500 nucleotides in length and sufficient to specifically hybridize under stringent conditions to 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893,33230, 1586, 9943, 16334, 68862, 9011, 14031, 6178, 21225, 1420, 32236, 2099, 2150, 26583, 2784, 8941, 9811, 27444, 50566 or 66428 mRNA or genomic DNA. Other suitable probes for use in the diagnostic assays of the invention are described herein.

A preferred agent for detecting 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein in a sample is an antibody capable of binding to 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein, preferably an antibody with a detectable label. Antibodies can be polyclonal, or more preferably, monoclonal. An intact antibody, or a fragment thereof (e.g., Fab or F(ab′)2) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin.

The term “biological sample” is intended to include tissues, cells, and biological fluids isolated from a subject, as well as tissues, cells, and fluids present within a subject. That is, the detection method of the invention can be used to detect 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 mRNA, protein, or genomic DNA in a biological sample in vitro as well as in vivo. For example, in vitro techniques for detection of 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 mRNA include Northern hybridizations and in situ hybridizations. In vitro techniques for detection of 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein include enzyme linked immunosorbent assays (ELISAs), Western blots, immunoprecipitations and immunofluorescence. In vitro techniques for detection of 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 genomic DNA include Southern hybridizations. Furthermore, in vivo techniques for detection of 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein include introducing into a subject a labeled anti-140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 antibody. For example, the antibody can be labeled with a radioactive marker whose presence and location in a subject can be detected by standard imaging techniques.

In another embodiment, the methods further involve obtaining a control biological sample from a control subject, contacting the control sample with a compound or agent capable of detecting 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein, mRNA, or genomic DNA, such that the presence of 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein, mRNA or genomic DNA is detected in the biological sample, and comparing the presence of 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein, mRNA or genomic DNA in the control sample with the presence of 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein, mRNA or genomic DNA in the test sample.

Prognostic Assays

The present invention further pertains to methods for identifying subjects having or at risk of developing a disease associated with aberrant 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 expression or activity.

As used herein, the term “aberrant” includes a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 expression or activity which deviates from the wild type 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 expression or activity. Aberrant expression or activity includes increased or decreased expression or activity, as well as expression or activity which does not follow the wild type developmental pattern of expression or the subcellular pattern of expression. For example, aberrant 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 expression or activity is intended to include the cases in which a mutation in the 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 gene causes the 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 gene to be under-expressed or over-expressed and situations in which such mutations result in a non-functional 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein or a protein which does not function in a wild-type fashion, e.g., a protein which does not interact with a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 substrate, or one which interacts with a non-140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 substrate.

The assays described herein, such as the preceding diagnostic assays or the following assays, can be used to identify a subject having or at risk of developing a disease. A biological sample may be obtained from a subject and tested for the presence or absence of a genetic alteration. For example, such genetic alterations can be detected by ascertaining the existence of at least one of 1) a deletion of one or more nucleotides from a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 gene, 2) an addition of one or more nucleotides to a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 gene, 3) a substitution of one or more nucleotides of a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 gene, 4) a chromosomal rearrangement of a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 gene, 5) an alteration in the level of a messenger RNA transcript of a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 gene, 6) aberrant modification of a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 gene, such as of the methylation pattern of the genomic DNA, 7) the presence of a non-wild type splicing pattern of a messenger RNA transcript of a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444,50566 or 66428 gene, 8) a non-wild type level of a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230,1586, 9943, 16334, 68862, 9011, 14031, 6178, 21225, 1420, 32236, 2099, 2150, 26583, 2784, 8941, 9811, 27444, 50566 or 66428-protein, 9) allelic loss of a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 gene, and 10) inappropriate post-translational modification of a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428-protein.

As described herein, there are a large number of assays known in the art which can be used for detecting genetic alterations in a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 gene. For example, a genetic alteration in a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 gene may be detected using a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) Proc. Natl. Acad. Sci. USA 91:360-364), the latter of which can be-particularly useful for detecting point mutations in a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 gene (see Abravaya et al. (1995) Nucleic Acids Res. 23:675-682). This method includes collecting a biological sample from a subject, isolating nucleic acid (e.g., genomic DNA, mRNA or both) from the sample, contacting the nucleic acid sample with one or more primers which specifically hybridize to a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 gene under conditions such that hybridization and amplification of the 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (Guatelli, J. C. et al. (1990) Proc. Natl. Acad. Sci. USA 87:1874-1878), transcriptional amplification system (Kwoh, D. Y. et al. (1989) Proc. Natl. Acad. Sci. USA 86:1173-1177), Q-Beta Replicase (Lizardi, P. M. et al. (1988) Bio-Technology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 gene from a biological sample can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, for example, U.S. Pat. No. 5,498,531) can be used to score for the presence of specific mutations by development or loss of a ribozyme cleavage site.

In other embodiments, genetic mutations in 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 can be identified by hybridizing biological sample derived and control nucleic acids, e.g., DNA or RNA, to high density arrays containing hundreds or thousands of oligonucleotide probes (Cronin, M. T. et al. (1996) Human Mutation 7:244-255; Kozal, M. J. et al. (1996) Nature Medicine 2:753-759). For example, genetic mutations in 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893,33230, 1586, 9943, 16334, 68862, 9011, 14031, 6178, 21225, 1420, 32236, 2099, 2150, 26583, 2784, 8941, 9811, 27444, 50566 or 66428 can be identified in two dimensional arrays containing light-generated DNA probes as described in Cronin, M. T. et al. (1996) supra. Briefly, a first hybridization array of probes can be used to scan through long stretches of DNA in a sample and control to identify base changes between the sequences by making linear arrays of sequential, overlapping probes. This step allows for the identification of point mutations. This step is followed by a second hybridization array that allows for the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 gene in a biological sample and detect mutations by comparing the sequence of the 140, 1470, 1686, 2089, 2427, 3702, 5891, 6428, 7181, 7660, 25641, 69583, 49863, 8897, 1682, 17667, 9235, 3703, 14171, 10359, 1660,1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 in the biological sample with the corresponding wild-type (control) sequence. Examples of sequencing reactions include those based on techniques developed by Maxam and Gilbert (1977) Proc. Natl. Acad. Sci. USA 74:560) or Sanger (1977) Proc. Natl. Acad. Sci. USA 74:5463). It is also contemplated that any of a variety of automated sequencing procedures can be utilized when performing the diagnostic assays (Naeve, C. W. (1995) Biotechniques 19:448-53), including sequencing by mass spectrometry (see, e.g., PCT International Publication No. WO 94/16101; Cohen et al. (1996) Adv. Chromatogr. 36:127-162; and Griffin et al. (1993) Appl. Biochem. Biotechnol. 38:147-159).

Other methods for detecting mutations in the 140, 1470, 1686, 2089, 2427, 3702,5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 gene include methods in which protection from cleavage agents is used to detect mismatched bases in RNA/RNA or RNA/DNA heteroduplexes (Myers et al. (1985) Science 230:1242). In general, the art technique of “mismatch cleavage” starts by providing heteroduplexes formed by hybridizing (labeled) RNA or DNA containing the wild-type 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 sequence with potentially mutant RNA or DNA obtained from a tissue sample. The double-stranded duplexes are treated with an agent which cleaves single-stranded regions of the duplex such as which will exist due to basepair mismatches between the control and sample strands. For instance, RNA/DNA duplexes can be treated with RNase and DNA/DNA hybrids treated with S1 nuclease to enzymatically digest the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, for example, Cotton et al. (1988) Proc. Natl Acad Sci USA 85:4397 and Saleeba et al. (1992) Methods Enzymol. 217:286-295. In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 cDNAs obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662). According to an exemplary embodiment, a probe based on a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 sequence, e.g., a wild-type 140, 1470, 1686, 2089, 2427, 3702,5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 sequence, is hybridized to a cDNA or other DNA product from a test cell(s). The duplex is treated with a DNA mismatch repair enzyme, and the cleavage products, if any, can be detected from electrophoresis protocols or the like. See, for example, U.S. Pat. No. 5,459,039.

In other embodiments, alterations in electrophoretic mobility will be used to identify mutations in 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 genes. For example, single strand conformation polymorphism (SSCP) may be used to detect differences in electrophoretic mobility between mutant and wild type nucleic acids (Orita et al. (1989) Proc Natl. Acad. Sci USA: 86:2766; see also Cotton (1993) Mutat. Res. 285:125-144 and Hayashi (1992) Genet. Anal. Tech. Appl. 9:73-79). Single-stranded DNA fragments of sample and control 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet 7:5).

In yet another embodiment the movement of mutant or wild-type fragments in polyacrylamide gels containing a gradient of denaturant is assayed using denaturing gradient gel electrophoresis (DGGE) (Myers et al. (1985) Nature 313:495). When DGGE is used as the method of analysis, DNA will be modified to ensure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys Chem 265:12753).

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions which permit hybridization only if a perfect match is found (Saiki et al. (1986) Nature 324:163); Saiki et al. (1989) Proc. Natl Acad. Sci USA 86:6230). Such allele specific oligonucleotides are hybridized to PCR amplified target DNA or a number of different mutations when the oligonucleotides are attached to the hybridizing membrane and hybridized with labeled target DNA.

Alternatively, allele specific amplification technology which depends on selective PCR amplification may be used in conjunction with the instant invention. Oligonucleotides used as primers for specific amplification may carry the mutation of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238). In addition it may be desirable to introduce a novel restriction site in the region of the mutation to create cleavage-based detection (Gasparini et al. (1992) Mol. Cell Probes 6: 1). It is anticipated that in certain embodiments amplification may also be performed using Taq ligase for amplification (Barany (1991) Proc. Natl. Acad. Sci USA 88:189). In such cases, ligation will occur only if there is a perfect match at the 3′ end of the 5′ sequence making it possible to detect the presence of a known mutation at a specific site by looking for the presence or absence of amplification.

Furthermore, the prognostic assays described herein can be used to determine whether a subject can be administered a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 modulator (e.g., an agonist, antagonist, peptidomimetic, protein, peptide, nucleic acid, or small molecule) to effectively treat a disease.

Monitoring of Effects During Clinical Trials

The present invention further provides methods for determining the effectiveness of a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 modulator (e.g., a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 modulator identified herein) in treating a disease. For example, the effectiveness of a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444,50566 or 66428 modulator in increasing 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 gene expression, protein levels, or in upregulating 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 activity, can be monitored in clinical trials of subjects exhibiting decreased 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 gene expression, protein levels, or downregulated 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 activity. Alternatively, the effectiveness of a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 modulator in decreasing 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 gene expression, protein levels, or in downregulating 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178,21225, 1420, 32236, 2099, 2150, 26583, 2784, 8941, 9811, 27444, 50566 or 66428 activity, can be monitored in clinical trials of subjects exhibiting increased 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 gene expression, protein levels, or 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 activity. In such clinical trials, the expression or activity of a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 gene, and preferably, other genes that have been implicated in nociception can be used as a “read out” or marker of the phenotype of a particular cell.

For example, and not by way of limitation, genes, including 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428, that are modulated in cells by treatment with an agent which modulates 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 activity (e.g., identified in a screening assay as described herein) can be identified. Thus, to study the effect of agents which modulate 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 activity on subjects suffering from a cancer in, for example, a clinical trial, cells can be isolated and RNA prepared and analyzed for the levels of expression of 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 and other genes implicated in the cancer. The levels of gene expression (e.g., a gene expression pattern) can be quantified by Northern blot analysis or RT-PCR, as described herein, or alternatively by measuring the amount of protein produced, by one of the methods described herein, or by measuring the levels of activity of 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893,33230, 1586, 9943, 16334, 68862, 9011, 14031, 6178, 21225, 1420, 32236, 2099, 2150, 26583, 2784, 8941, 9811, 27444, 50566 or 66428 or other genes. In this way, the gene expression pattern can serve as a marker, indicative of the physiological response of the cells to the agent which modulates 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 activity. This response state may be determined before, and at various points during treatment of the individual with the agent which modulates 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 activity.

In a preferred embodiment, the present invention provides a method for monitoring the effectiveness of treatment of a subject with an agent which modulates 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 activity (e.g., an agonist, antagonist, peptidomimetic, protein, peptide, nucleic acid, or small molecule identified by the screening assays described herein) including the steps of (i) obtaining a pre-administration sample from a subject prior to administration of the agent; (ii) detecting the level of expression of a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein, mRNA, or genomic DNA in the pre-administration sample; (iii) obtaining one or more post-administration samples from the subject; (iv) detecting the level of expression or activity of the 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein, mRNA, or genomic DNA in the post-administration samples; (v) comparing the level of expression or activity of the 140, 1470, 1686, 2089, 2427, 3702, 5891, 6428, 7181,7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein, mRNA, or genomic DNA in the pre-administration sample with the 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein, mRNA, or genomic DNA in the post administration sample or samples; and (vi) altering the administration of the agent to the subject accordingly. For example, increased administration of the agent may be desirable to increase the expression or activity of 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 to higher levels than detected, i.e., to increase the effectiveness of the agent. Alternatively, decreased administration of the agent may be desirable to decrease expression or activity of 140, 1470, 1686, 2089, 2427, 3702, 5891, 6428, 7181,7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 to lower levels than detected, i.e. to decrease the effectiveness of the agent. According to such an embodiment, 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 expression or activity may be used as an indicator of the effectiveness of an agent, even in the absence of an observable phenotypic response.

Methods of Treatment:

The present invention provides for both prophylactic and therapeutic methods of treating a subject, e.g., a human, at risk of (or susceptible to) a disease. With regard to both prophylactic and therapeutic methods of treatment, such treatments may be specifically tailored or modified, based on knowledge obtained from the field of pharmacogenomics. “Pharmacogenomics,” as used herein, refers to the application of genomics technologies such as gene sequencing, statistical genetics, and gene expression analysis to drugs in clinical development and on the market. More specifically, the term refers to the study of how a patient's genes determine his or her response to a drug (e.g., a patient's “drug response phenotype”, or “drug response genotype”).

Thus, another aspect of the invention provides methods for tailoring an subject's prophylactic or therapeutic treatment with either the 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 molecules of the present invention or 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 modulators according to that individual's drug response genotype. Pharmacogenomics allows a clinician or physician to target prophylactic or therapeutic treatments to patients who will most benefit from the treatment and to avoid treatment of patients who will experience toxic drug-related side effects.

Prophylactic Methods

In one aspect, the invention provides a method for preventing in a subject, a disease by administering to the subject an agent which modulates 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 expression or 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297,1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 activity. Subjects at risk for a cancer, e.g., lung, colon, prostate, ovarian or breast cancer, can be identified by, for example, any or a combination of the diagnostic or prognostic assays described herein. Administration of a prophylactic agent can occur prior to the manifestation of symptoms characteristic of aberrant 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 expression or activity, such that a disease is prevented or, alternatively, delayed in its progression. Depending on the type of 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 aberrancy, for example, a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428, 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 agonist or 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 antagonist agent can be used for treating the subject. The appropriate agent can be determined based on screening assays described herein.

Therapeutic Methods

Described herein are methods and compositions whereby a cancer may be ameliorated. Certain cancers are brought about, at least in part, by an excessive level of a gene product, or by the presence of a gene product exhibiting an abnormal or excessive activity. As such, the reduction in the level and/or activity of such gene products would bring about the amelioration of at least one symptom of a cancer. Techniques for the reduction of gene expression levels or the activity of a protein are discussed below.

Alternatively, certain other cancer are brought about, at least in part, by the absence or reduction of the level of gene expression, or a reduction in the level of a protein's activity. As such, an increase in the level of gene expression and/or the activity of such proteins would bring about the amelioration of at least one symptom of a cancer.

In some cases, the up-regulation of a gene in a disease state reflects a protective role for that gene product in responding to the disease condition. Enhancement of such a gene's expression, or the activity of the gene product, will reinforce the protective effect it exerts. Some urological disease states may result from an abnormally low level of activity of such a protective gene. In these cases also, an increase in the level of gene expression and/or the activity of such gene products would bring about the amelioration of a least one symptom of a cancer. Techniques for increasing target gene expression levels or target gene product activity levels are discussed herein.

Accordingly, another aspect of the invention pertains to methods of modulating 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 expression or activity for therapeutic purposes. Accordingly, in an exemplary embodiment, the modulatory method of the invention involves contacting a cell with a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 or agent that modulates one or more of the activities of 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein activity associated with the cell (e.g., an endothelial cell, ovarian cell, bladder cell and prostate cell). An agent that modulates 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein activity can be an agent as described herein, such as a nucleic acid or a protein, a naturally-occurring target molecule of a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein (e.g., a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 ligand or substrate), a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584,9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 antibody, a 140, 1470, 1686, 2089, 2427, 3702, 5891, 6428, 7181,7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 agonist or antagonist, a peptidomimetic of a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 agonist or antagonist, or other small molecule. In one embodiment, the agent stimulates one or more 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 activities. Examples of such stimulatory agents include active 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein and a nucleic acid molecule encoding 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 that has been introduced into the cell. In another embodiment, the agent inhibits one or more 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 activities. Examples of such inhibitory agents include antisense 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 nucleic acid molecules, anti-140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 antibodies, and 140, 1470, 1686, 2089, 2427, 3702, 5891, 6428, 7181, 7660, 25641, 69583, 49863, 8897, 1682, 17667, 9235, 3703, 14171, 10359, 1660,1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 inhibitors. These modulatory methods can be performed in vitro (e.g., by culturing the cell with the agent) or, alternatively, in vivo (e.g., by administering the agent to a subject). As such, the present invention provides methods of treating an individual afflicted with a disease or disorder characterized by aberrant or unwanted expression or activity of a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein or nucleic acid molecule. In one embodiment, the method involves administering an agent (e.g., an agent identified by a screening assay described herein), or combination of agents that modulates (e.g., upregulates or downregulates) 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 expression or activity. In another embodiment, the method involves administering a 140, 1470, 1686, 2089, 2427, 3702,5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein or nucleic acid molecule as therapy to compensate for reduced, aberrant, or unwanted 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 expression or activity.

Stimulation of 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 activity is desirable in situations in which 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 is abnormally downregulated and/or in which increased 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444,50566 or 66428 activity is likely to have a beneficial effect. Likewise, inhibition of 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 activity is desirable in situations in which 140, 1470, 1686, 2089, 2427, 3702,5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 is abnormally upregulated and/or in which decreased 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 activity is likely to have a beneficial effect.

Methods for Inhibiting Target Gene Expression, Synthesis, or Activity

As discussed above, genes involved in cancer may cause such disorders via an increased level of gene activity. In some cases, such up-regulation may have a causative or exacerbating effect on the disease state. A variety of techniques may be used to inhibit the expression, synthesis, or activity of such genes and/or proteins.

For example, compounds such as those identified through assays described above, which exhibit inhibitory activity, may be used in accordance with the invention to ameliorate at least one symptom of a cancer. Such molecules may include, but are not limited to, small organic molecules, peptides, antibodies, and the like.

For example, compounds can be administered that compete with endogenous ligand for the 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein. The resulting reduction in the amount of ligand-bound 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein will modulate endothelial cell physiology. Compounds that can be particularly useful for this purpose include, for example, soluble proteins or peptides, such as peptides comprising one or more of the extracellular domains, or portions and/or analogs thereof, of the 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein, including, for example, soluble fusion proteins such as Ig-tailed fusion proteins. (For a discussion of the production of Ig-tailed fusion proteins, see, for example, U.S. Pat. No. 5,116,964). Alternatively, compounds, such as ligand analogs or antibodies, that bind to the 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893,33230, 1586, 9943, 16334, 68862, 9011, 14031, 6178, 21225, 1420, 32236, 2099, 2150, 26583, 2784, 8941, 9811, 27444, 50566 or 66428 receptor site, but do not activate the protein, (e.g., receptor-ligand antagonists) can be effective in inhibiting 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein activity.

Further, antisense and ribozyme molecules which inhibit expression of the 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 gene may also be used in accordance with the invention to inhibit aberrant 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 gene activity. Still further, triple helix molecules may be utilized in inhibiting aberrant 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 gene activity.

The antisense nucleic acid molecules used in the methods of the invention are typically administered to a subject or generated in situ such that they hybridize with or bind to cellular mRNA and/or genomic DNA encoding a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein to thereby inhibit expression of the protein, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. An example of a route of administration of antisense nucleic acid molecules of the invention include direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

In yet another embodiment, an antisense nucleic acid molecule used in the methods of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids. Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

In still another embodiment, an antisense nucleic acid used in the methods of the invention is a ribozyme. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes (described in Haselhoff and Gerlach (1988) Nature 334:585-591)) can be used to catalytically cleave 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 mRNA transcripts to thereby inhibit translation of 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 mRNA. A ribozyme having specificity for a 32457-encoding nucleic acid can be designed based upon the nucleotide sequence of a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 cDNA disclosed herein (i.e., SEQ ID NO:1, 3,.5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119 or 121). For example, a derivative of a Tetrahymena L-19 IVS RNA can be constructed in which the nucleotide sequence of the active site is complementary to the nucleotide sequence to be cleaved in a, 2784, 8941, 9811, 27444, 50566 or 66428-encoding mRNA (see, for example, Cech et al. U.S. Pat. No. 4,987,071; and Cech et al. U.S. Pat. No. 5,116,742). Alternatively, 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 mRNA can be used to select a catalytic RNA having a specific ribonuclease activity from a pool of RNA molecules (see, for example, Bartel, D. and Szostak, J. W. (1993) Science 261:1411-1418).

140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 gene expression can also be inhibited by targeting nucleotide sequences complementary to the regulatory region of the 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893,33230, 1586, 9943, 16334, 68862, 9011, 14031, 6178, 21225, 1420, 32236, 2099, 2150, 26583, 2784, 8941, 9811, 27444, 50566 or 66428 (e.g., the 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 promoter and/or enhancers) to form triple helical structures that prevent transcription of the 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 gene in target cells (see, for example, Helene, C. (1991) Anticancer Drug Des. 6(6):569-84; Helene, C. et al. (1992) Ann. N.Y. Acad. Sci. 660:27-36; and Maher, L. J. (1992) Bioassays 14(12):807-15).

Antibodies that are both specific for the 140, 1470, 1686, 2089, 2427, 3702, 5891, 6428, 7181, 7660, 25641, 69583, 49863, 8897, 1682, 17667, 9235, 3703,14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein and interfere with its activity may also be used to modulate or inhibit 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein function. Such antibodies may be generated using standard techniques described herein, against the 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein itself or against peptides corresponding to portions of the protein. Such antibodies include but are not limited to polyclonal, monoclonal, Fab fragments, single chain antibodies, or chimeric antibodies.

In instances where the target gene protein is intracellular and whole antibodies are used, internalizing antibodies may be preferred. Lipofectin liposomes may be used to deliver the antibody or a fragment of the Fab region which binds to the target epitope into cells. Where fragments of the antibody are used, the smallest inhibitory fragment which binds to the target protein's binding domain is preferred. For example, peptides having an amino acid sequence corresponding to the domain of the variable region of the antibody that binds to the target gene protein may be used. Such peptides may be synthesized chemically or produced via recombinant DNA technology using methods well known in the art (described in, for example, Creighton (1983), supra; and Sambrook et al. (1989) supra). Single chain neutralizing antibodies which bind to intracellular target gene epitopes may also be administered. Such single chain antibodies may be administered, for example, by expressing nucleotide sequences encoding single-chain antibodies within the target cell population by utilizing, for example, techniques such as those described in Marasco et al. (1993) Proc. Natl. Acad. Sci. USA 90:7889-7893).

In some instances, the target gene protein is extracellular, or is a transmembrane protein, such as the 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein. Antibodies that are specific for one or more extracellular domains of the 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein, for example, and that interfere with its activity, are particularly useful in treating cancer or a cancer. Such antibodies are especially efficient because they can access the target domains directly from the bloodstream. Any of the administration techniques described below which are appropriate for peptide administration may be utilized to effectively administer inhibitory target gene antibodies to their site of action.

Methods for Restoring or Enhancing Target Gene Activity

Genes that cause a cancer may be underexpressed within the cancer. Alternatively, the activity of the protein products of such genes may be decreased, leading to the development of cancer. Such down-regulation of gene expression or decrease of protein activity might have a causative or exacerbating effect on the disease state.

In some cases, genes that are up-regulated in the disease state might be exerting a protective effect. A variety of techniques may be used to increase the expression, synthesis, or activity of genes and/or proteins that exert a protective effect in response to a cancer.

Described in this section are methods whereby the level of 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 activity may be increased to levels wherein the symptoms of the cancer are ameliorated. The level of 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 activity may be increased, for example, by either increasing the level of 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 gene expression or by increasing the level of active 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein which is present.

For example, a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein, at a level sufficient to ameliorate at least one symptom of a cancer may be administered to a patient exhibiting such symptoms. Any of the techniques discussed below may be used for such administration. One of skill in the art will readily know how to determine the concentration of effective, non-toxic doses of the 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein, utilizing techniques such as those described below.

Additionally, RNA sequences encoding a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein may be directly administered to a patient exhibiting a cancer, at a concentration sufficient to produce a level of 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein such that a cancer are ameliorated. Any of the techniques discussed below, which achieve intracellular administration of compounds, such as, for example, liposome administration, may be used for the administration of such RNA molecules. The RNA molecules may be produced, for example, by recombinant techniques such as those described herein.

Further, subjects may be treated by gene replacement therapy. One or more copies of a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 gene, or a portion thereof, that directs the production of a normal 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein with 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 function, may be inserted into cells using vectors which include, but are not limited to adenovirus, adeno-associated virus, and retrovirus vectors, in addition to other particles that introduce DNA into cells, such as liposomes. Additionally, techniques such as those described above may be used for the introduction of 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 gene sequences into human cells.

Cells, preferably, autologous cells, containing 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 expressing gene sequences may then be introduced or reintroduced into the subject at positions which allow for the amelioration of at least one symptom of a cancer. Such cell replacement techniques may be preferred, for example, when the gene product is a secreted, extracellular gene product.

Pharmaceutical Compositions

Another aspect of the invention pertains to methods for treating a subject suffering from a disease. These methods involve administering to a subject an agent which modulates 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 expression or activity (e.g., an agent identified by a screening assay described herein), or a combination of such agents. In another embodiment, the method involves administering to a subject a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein or nucleic acid molecule as therapy to compensate for reduced, aberrant, or unwanted 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 expression or activity.

Stimulation of 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 activity is desirable in situations in which 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 is abnormally downregulated and/or in which increased 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 activity is likely to have a beneficial effect. Likewise, inhibition of 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 activity is desirable in situations in which 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 is abnormally upregulated and/or in which decreased 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 activity is likely to have a beneficial effect.

The agents which modulate 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 activity can be administered to a subject using pharmaceutical compositions suitable for such administration. Such compositions typically comprise the agent (e.g., nucleic acid molecule, protein, or antibody) and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition used in the therapeutic methods of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the agent that modulates 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 activity (e.g., a fragment of a. 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein or an anti-140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The agents that modulate 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 activity can also be prepared in the form of suppositories (e.g., with conventional suppository bases such as cocoa butter and other glycerides) or retention enemas for rectal delivery.

In one embodiment, the agents that modulate 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 activity are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the agent that modulates 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 activity and the particular therapeutic effect to be achieved, and the limitations inherent in the art of compounding such an agent for the treatment of subjects.

Toxicity and therapeutic efficacy of such agents can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and can be expressed as the ratio LD50/ED50. Agents which exhibit large therapeutic indices are preferred. While agents that exhibit toxic side effects may be used, care should be taken to design a delivery system that targets such agents to the site of affected tissue in order to minimize potential damage to uninfected cells and, thereby, reduce side effects.

The data obtained from the cell culture assays and animal studies can be used in formulating a range of dosage for use in humans. The dosage of such 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 modulating agents lies preferably within a range of circulating concentrations that include the ED50 with little or no toxicity. The dosage may vary within this range depending upon the dosage form employed and the route of administration utilized. For any agent used in the therapeutic methods of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of protein or polypeptide (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a protein, polypeptide, or antibody can include a single treatment or, preferably, can include a series of treatments.

In a preferred example, a subject is treated with antibody, protein, or polypeptide in the range of between about 0.1 to 20 mg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of antibody, protein, or polypeptide used for treatment may increase or decrease over the course of a particular treatment. Changes in dosage may result and become apparent from the results of diagnostic assays as described herein.

The present invention encompasses agents which modulate expression or activity. An agent may, for example, be a small molecule. For example, such small molecules include, but are not limited to, peptides, peptidomimetics, amino acids, amino acid analogs, polynucleotides, polynucleotide analogs, nucleotides, nucleotide analogs, organic or inorganic compounds (i.e,. including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds. It is understood that appropriate doses of small molecule agents depends upon a number of factors within the ken of the ordinarily skilled physician, veterinarian, or researcher. The dose(s) of the small molecule will vary, for example, depending upon the identity, size, and condition of the subject or sample being treated, further depending upon the route by which the composition is to be administered, if applicable, and the effect which the practitioner desires the small molecule to have upon the nucleic acid or polypeptide of the invention.

Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram). It is furthermore understood that appropriate doses of a small molecule depend upon the potency of the small molecule with respect to the expression or activity to be modulated. Such appropriate doses may be determined using the assays described herein. When one or more of these small molecules is to be administered to an animal (e.g., a human) in order to modulate expression or activity of a polypeptide or nucleic acid of the invention, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

Further, an antibody (or fragment thereof) may be conjugated to a therapeutic moiety such as a cytotoxin, a therapeutic agent or a radioactive metal ion. A cytotoxin or cytotoxic agent includes any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

The conjugates of the invention can be used for modifying a given biological response, the drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, alpha-interferon, beta-interferon, nerve growth factor, platelet derived growth factor, tissue plasminogen activator; or biological response modifiers such as, for example, lymphokines, interleukin-1 (“IL-1”), interleukin-2 (“IL-2”), interleukin-6 (“IL-6”), granulocyte macrophase colony stimulating factor (“GM-CSF”), granulocyte colony stimulating factor (“G-CSF”), or other growth factors.

Techniques for conjugating such therapeutic moiety to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2^{nd} Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”,. Immunol. Rev., 62:119-58 (1982). Alternatively, an antibody can be conjugated to a second antibody to form an antibody heteroconjugate as described by Segal in U.S. Pat. No. 4,676,980.

The nucleic acid molecules used in the methods of the invention can be inserted into vectors and used as gene therapy vectors. Gene therapy vectors can be delivered to a subject by, for example, intravenous injection, local administration (see U.S. Pat. No. 5,328,470) or by stereotactic injection (see, e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

Pharmacogenomics

In conjunction with the therapeutic methods of the invention, pharmacogenomics (i.e., the study of the relationship between a subject's genotype and that subject's response to a foreign compound or drug) may be considered. Differences in metabolism of therapeutics can lead to severe toxicity or therapeutic failure by altering the relation between dose and blood concentration of the pharmacologically active drug. Thus, a physician or clinician may consider applying knowledge obtained in relevant pharmacogenomics studies in determining whether to administer an agent which modulates 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893,33230, 1586, 9943, 16334, 68862, 9011, 14031, 6178, 21225, 1420, 32236, 2099, 2150, 26583, 2784, 8941, 9811, 27444, 50566 or 66428 activity, as well as tailoring the dosage and/or therapeutic regimen of treatment with an agent which modulates 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 activity.

Pharmacogenomics deals with clinically significant hereditary variations in the response to drugs due to altered drug disposition and abnormal action in affected persons. See, for example, Eichelbaum, M. et al. (1996) Clin. Exp. Pharmacol. Physiol. 23(10-11): 983-985 and Linder, M. W. et al. (1997) Clin. Chem. 43(2):254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare genetic defects or as naturally-occurring polymorphisms. For example, glucose-6-phosphate aminopeptidase deficiency (G6PD) is a common inherited enzymopathy in which the main clinical complication is haemolysis after ingestion of oxidant drugs (anti-malarials, sulfonamides, analgesics, nitrofurans) and consumption of fava beans.

One pharmacogenomics approach to identifying genes that predict drug response, known as “a genome-wide association”, relies primarily on a high-resolution map of the human genome consisting of already known gene-related markers (e.g., a “bi-allelic” gene marker map which consists of 60,000-100,000 polymorphic or variable sites on the human genome, each of which has two variants). Such a high-resolution genetic map can be compared to a map of the genome of each of a statistically significant number of patients taking part in a Phase II/III drug trial to identify markers associated with a particular observed drug response or side effect. Alternatively, such a high resolution map can be generated from a combination of some ten million known single nucleotide polymorphisms (SNPs) in the human genome. As used herein, a “SNP” is a common alteration that occurs in a single nucleotide base in a stretch of DNA. For example, a SNP may occur once per every 1000 bases of DNA. A SNP may be involved in a disease process, however, the vast majority may not be disease-associated. Given a genetic map based on the occurrence of such SNPs, individuals can be grouped into genetic categories depending on a particular pattern of SNPs in their individual genome. In such a manner, treatment regimens can be tailored to groups of genetically similar individuals, taking into account traits that may be common among such genetically similar individuals.

Alternatively, a method termed the “candidate gene approach” can be utilized to identify genes that predict drug response. According to this method, if a gene that encodes a drug target is known (e.g., a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein used in the methods of the present invention), all common variants of that gene can be fairly easily identified in the population and it can be determined if having one version of the gene versus another is associated with a particular drug response.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and the cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. The other extreme are the so called ultra-rapid metabolizers who do not respond to standard doses. Recently, the molecular basis of ultra-rapid metabolism has been identified to be due to CYP2D6 gene amplification.

Alternatively, a method termed the “gene expression profiling” can be utilized to identify genes that predict drug response. For example, the gene expression of an animal dosed with a drug (e.g., a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 molecule or 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 modulator used in the methods of the present invention) can give an indication whether gene pathways related to toxicity have been turned on.

Information generated from more than one of the above pharmacogenomics approaches can be used to determine appropriate dosage and treatment regimens for prophylactic or therapeutic treatment of a subject. This knowledge, when applied to dosing or drug selection, can avoid adverse reactions or therapeutic failure and, thus, enhance therapeutic or prophylactic efficiency when treating a subject suffering from a cancer, with an agent which modulates 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 activity.

Recombinant Expression Vectors and Host Cells Used in the Methods of the Invention

The methods of the invention (e.g., the screening assays described herein) include the use of vectors, preferably expression vectors, containing a nucleic acid encoding a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein (or a portion thereof). As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors to be used in the methods of the invention comprise a nucleic acid of the invention in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel (1990) Methods Enzymol. 185:3-7. Regulatory sequences include those which direct constitutive expression of a nucleotide sequence in many types of host cells and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, and the like. The expression vectors of the invention can be introduced into host cells to thereby produce proteins or peptides, including fusion proteins or peptides, encoded by nucleic acids as described herein (e.g., 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 proteins, mutant forms of 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 proteins, fusion proteins, and the like).

The recombinant expression vectors to be used in the methods of the invention can be designed for expression of 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 proteins in prokaryotic or eukaryotic cells. For example, 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 proteins can be expressed in bacterial cells such as E. coli, insect cells (using baculovirus expression vectors), yeast cells, or mammalian cells. Suitable host cells are discussed further in Goeddel (1990) supra. Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith, D. B. and Johnson, K. S. (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Purified fusion proteins can be utilized in 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 activity assays, (e.g., direct assays or competitive assays described in detail below), or to generate antibodies specific for 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 proteins. In a preferred embodiment, a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784,8941, 9811, 27444, 50566 or 66428 fusion protein expressed in a retroviral expression vector of the present invention can be utilized to infect bone marrow cells which are subsequently transplanted into irradiated recipients. The pathology of the subject recipient is then examined after sufficient time has passed (e.g., six weeks).

In another embodiment, a nucleic acid of the invention is expressed in mammalian cells using a mammalian expression vector. Examples of mammalian expression vectors include pCDM8 (Seed, B. (1987) Nature 329:840) and pMT2PC (Kaufman et al. (1987) EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook, J. et al., Molecular Cloning: A Laboratory Manual. 2^{nd} ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid).

The methods of the invention may further use a recombinant expression vector comprising a DNA molecule of the invention cloned into the expression vector in an antisense orientation. That is, the DNA molecule is operatively linked to a regulatory sequence in a manner which allows for expression (by transcription of the DNA molecule) of an RNA molecule which is antisense to 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 mRNA. Regulatory sequences operatively linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue specific, or cell type specific expression of antisense RNA. The antisense expression vector can be in the form of a recombinant plasmid, phagemid, or attenuated virus in which antisense nucleic acids are produced under the control of a high efficiency regulatory region, the activity of which can be determined by the cell type into which the vector is introduced. For a discussion of the regulation of gene expression using antisense genes, see Weintraub, H. et al., Antisense RNA as a molecular tool for genetic analysis, Reviews—Trends in Genetics, Vol. 1(1) 1986.

Another aspect of the invention pertains to the use of host cells into which a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 nucleic acid molecule of the invention is introduced, e.g., a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 nucleic acid molecule within a recombinant expression vector or a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 nucleic acid molecule containing sequences which allow it to homologously recombine into a specific site of the host cell's genome. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook et al. (Molecular Cloning: A Laboratory Manual. 2^{nd}, ed., Cold Spring Harbor Laboratory, Cold Spring Harbor Laboratory Press, Cold Spring Harbor, N.Y., 1989), and other laboratory manuals.

A host cell used in the methods of the invention, such as a prokaryotic or eukaryotic host cell in culture, can be used to produce (i.e., express) a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein. Accordingly, the invention further provides methods for producing a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297,1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein using the host cells of the invention. In one embodiment, the method comprises culturing the host cell of the invention (into which a recombinant expression vector encoding a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein has been introduced) in a suitable medium such that a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, .14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein is produced. In another embodiment, the method further comprises isolating a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein from the medium or the host cell.

Isolated Nucleic Acid Molecules Used in the Methods of the Invention

The methods of the invention include the use of isolated nucleic acid molecules that encode 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 proteins or biologically active portions thereof, as well as nucleic acid fragments sufficient for use as hybridization probes to identify 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428-encoding nucleic acid molecules (e.g., 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 mRNA) and fragments for use as PCR primers for the amplification or mutation of 140, 1470, 1686, 2089, 2427, 3702,5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 nucleic acid molecules. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

A nucleic acid molecule used in the methods of the present invention, e.g., a nucleic acid molecule having the nucleotide sequence of SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119 or 121, or a portion thereof, can be isolated using standard molecular biology techniques and the sequence information provided herein. Using all or portion of the nucleic acid sequence of SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119 or 121, as a hybridization probe, 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 nucleic acid molecules can be isolated using standard hybridization and cloning techniques (e.g., as described in Sambrook, J., Fritsh, E. F., and Maniatis, T. Molecular Cloning: A Laboratory Manual. 2^{nd,} ed., Cold Spring Harbor Laboratory, Cold Spring Harbor Laboratory Press, Cold Spring Harbor, N.Y., 1989).

Moreover, a nucleic acid molecule encompassing all or a portion of SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119 or 121 can be isolated by the polymerase chain reaction (PCR) using synthetic oligonucleotide primers designed based upon the sequence of SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119 or 121.

A nucleic acid used in the methods of the invention can be amplified using cDNA, mRNA or, alternatively, genomic DNA as a template and appropriate oligonucleotide primers according to standard PCR amplification techniques. Furthermore, oligonucleotides corresponding to 140, 1470, 1686, 2089, 2427, 3702, 5891, 6428, 7181, 7660, 25641, 69583, 49863, 8897, 1682, 17667, 9235, 3703, 14171, 10359, 1660,1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 nucleotide sequences can be prepared by standard synthetic techniques, e.g., using an automated DNA synthesizer.

In a preferred embodiment, the isolated nucleic acid molecules used in the methods of the invention comprise the nucleotide sequence shown in SEQ ID NO:1, 3, 5, 7, 9, 11, 13, 15, 17, 19, 21, 23, 25, 27, 29, 31, 33, 35, 37, 39, 41, 43, 45, 47,49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119 or 121, a complement of the nucleotide sequence shown in SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119 or 121, or a portion of any of these nucleotide sequences. A nucleic acid molecule which is complementary to the nucleotide sequence shown in SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119 or 121, is one which is sufficiently complementary to the nucleotide sequence shown in SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119 or 121, such that it can hybridize to the nucleotide sequence shown in SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119 or 121, thereby forming a stable duplex.

In still another preferred embodiment, an isolated nucleic acid molecule used in the methods of the present invention comprises a nucleotide sequence which is at least about 55%, 60%, 65%, 70%, 75%, 80%, 85%, 90%, 95%, 96%, 97%, 98%, 99% or more identical to the entire length of the nucleotide sequence shown in SEQ ID NO:1, 3, 5, 7, 9, 11, 13, 15, 17, 19,21, 23, 25, 27, 29, 31, 33, 35, 37, 39,41,43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119 or 121, or a portion of any of this nucleotide sequence.

Moreover, the nucleic acid molecules used in the methods of the invention can comprise only a portion of the nucleic acid sequence of SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119 or 121, for example, a fragment which can be used as a probe or primer or a fragment encoding a portion of a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein, e.g., a biologically active portion of a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein. The probe/primer typically comprises substantially purified oligonucleotide. The oligonucleotide typically comprises a region of nucleotide sequence that hybridizes under stringent conditions to at least about 12 or 15, preferably about 20 or 25, more preferably about 30, 35, 40, 45, 50, 55, 60, 65, or 75 consecutive nucleotides of a sense sequence of SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119 or 121, of an anti-sense sequence of SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119 or 121, or of a naturally occurring allelic variant or mutant of SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119 or 121. In one embodiment, a nucleic acid molecule used in the methods of the present invention comprises a nucleotide sequence which is greater than 100, 100-200, 200-300, 300-400, 400-500, 500-600, 600-700, 700-800, 800-900, 900-1000, 1000-1100, 1100-1200, 1200-1300, or more nucleotides in length and hybridizes under stringent hybridization conditions to a nucleic acid molecule of SEQ ID NO:1, 3, 5,7, 9, 11, 13, 15, 17, 19, 21, 23, 25, 27, 29, 31, 33, 35, 37, 39, 41, 43, 45, 47,49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119 or 121.

As used herein, the term “hybridizes under stringent conditions” is intended to describe conditions for hybridization and washing under which nucleotide sequences that are significantly identical or homologous to each other remain hybridized to each other. Preferably, the conditions are such that sequences at least about 70%, more preferably at least about 80%, even more preferably at least about 85% or 90% identical to each other remain hybridized to each other. Such stringent conditions are known to those skilled in the art and can be found in Current Protocols in Molecular Biology, Ausubel et al., eds., John Wiley & Sons, Inc. (1995), sections 2, 4 and 6. Additional stringent conditions can be found in Molecular Cloning: A Laboratory Manual, Sambrook et al., Cold Spring Harbor Press, Cold Spring Harbor, N.Y. (1989), chapters 7, 9 and 11. A preferred, non-limiting example of stringent hybridization conditions includes hybridization in 4× sodium chloride/sodium citrate (SSC), at about 65-70° C. (or hybridization in 4×SSC plus 50% formamide at about 42-50° C.) followed by one or more washes in 1×SSC, at about 65-70° C. A preferred, non-limiting example of highly stringent hybridization conditions includes hybridization in 1×SSC, at about 65-70° C. (or hybridization in 1×SSC plus 50% formamide at about 42-50° C.) followed by one or more washes in 0.3×SSC, at about 65-70° C. A preferred, non-limiting example of reduced stringency hybridization conditions includes hybridization in 4×SSC, at about 50-60° C. (or alternatively hybridization in 6×SSC plus 50% formamide at about 40-45° C.) followed by one or more washes in 2×SSC, at about 50-60° C. Ranges intermediate to the above-recited values, e.g., at 65-70° C. or at 42-50° C. are also intended to be encompassed by the present invention. SSPE (1×SSPE is 0.15M NaCl, 10 mM NaH₂PO₄, and 1.25mM EDTA, pH 7.4) can be substituted for SSC (1×SSC is 0. 15M NaCl and 15mM sodium citrate) in the hybridization and wash buffers; washes are performed for 15 minutes each after hybridization is complete. The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, Tₘ(° C.)=2(# of A+T bases)+4(# of G+C bases). For hybrids between 18 and 49 base pairs in length, Tₘ(° C.)=81.5+16.6(log₁₀[Na⁺])+0.41(% G+C)−(600/N), where N is the number of bases in the hybrid, and [Na⁺] is the concentration of sodium ions in the hybridization buffer ([Na⁺] for 1×SSC=0.165 M). It will also be recognized by the skilled practitioner that additional reagents may be added to hybridization and/or wash buffers to decrease non-specific hybridization of nucleic acid molecules to membranes, for example, nitrocellulose or nylon membranes, including but not limited to blocking agents (e.g., BSA or salmon or herring sperm carrier DNA), detergents (e.g., SDS), chelating agents (e.g., EDTA), Ficoll, PVP and the like. When using nylon membranes, in particular, an additional preferred, non-limiting example of stringent hybridization conditions is hybridization in 0.25-0.5M NaH₂PO₄, 7% SDS at about 65° C., followed by one or more washes at 0.02M NaH₂PO₄, 1% SDS at 65° C., see e.g., Church and Gilbert (1984) Proc. Natl. Acad. Sci. USA 81:1991-1995, (or alternatively 0.2×SSC, 1% SDS).

In preferred embodiments, the probe further comprises a label group attached thereto, e.g., the label group can be a radioisotope, a fluorescent compound, an enzyme, or an enzyme co-factor. Such probes can be used as a part of a diagnostic test kit for identifying cells or tissue which misexpress a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein, such as by measuring a level of a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428-encoding nucleic acid in a sample of cells from a subject e.g., detecting 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 mRNA levels or determining whether a genomic 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 gene has been mutated or deleted.

The methods of the invention further encompass the use of nucleic acid molecules that differ from the nucleotide sequence shown in SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119 or 121, due to degeneracy of the genetic code and thus encode the same 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 proteins as those encoded by the nucleotide sequence shown in SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119 or 121. In another embodiment, an isolated nucleic acid molecule included in the methods of the invention has a nucleotide sequence encoding a protein having an amino acid sequence shown in SEQ ID NO:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120 or 122.

The methods of the invention further include the use of allelic variants of human 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428, e.g., functional and non-functional allelic variants. Functional allelic variants are naturally occurring amino acid sequence variants of the human 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein that maintain a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 activity. Functional allelic variants will typically contain only conservative substitution of one or more amino acids of SEQ ID NO:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94,96, 98, 100, 102, 104, 106, 108, 110, 112, 114, 116, 118, 120 or 122, or substitution, deletion or insertion of non-critical residues in non-critical regions of the protein.

Non-functional allelic variants are naturally occurring amino acid sequence variants of the human 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein that do not have a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230,1586, 9943, 16334, 68862, 9011, 14031, 6178, 21225, 1420, 32236, 2099, 2150, 26583, 2784, 8941, 9811, 27444, 50566 or 66428 activity. Non-functional allelic variants will typically contain a non-conservative substitution, deletion, or insertion or premature truncation of the amino acid sequence of SEQ ID NO:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120 or 122 or a substitution, insertion or deletion in critical residues or critical regions of the protein.

The methods of the present invention may further use non-human orthologues of the human 140, 1470, 1686, 2089, 2427, 3702, 5891, 6428, 7181, 7660, 25641, 69583, 49863, 8897, 1682, 17667, 9235, 3703, 14171, 10359, 1660,1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein. Orthologues of the human 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein are proteins that are isolated from non-human organisms and possess the same 140, 1470, 1686, 2089, 2427, 3702, 5891, 6428, 7181,7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 activity.

The methods of the present invention further include the use of nucleic acid molecules comprising the nucleotide sequence of SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119 or 121 or a portion thereof, in which a mutation has been introduced. The mutation may lead to amino acid substitutions at “non-essential” amino acid residues or at “essential” amino acid residues. A “non-essential” amino acid residue is a residue that can be altered from the wild-type sequence of 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 (e.g., the sequence of SEQ ID NO:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70,72, 74, 76, 78, 80, 82, 84, 86, 88, 90, 92, 94, 96, 98, 100, 102, 104, 106, 108, 110, 112, 114, 116, 118, 120 or 122) without altering the biological activity, whereas an “essential” amino acid residue is required for biological activity. For example, amino acid residues that are conserved among the 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 proteins of the present invention are not likely to be amenable to alteration.

Mutations can be introduced into SEQ ID NO:1, 3,5,7,9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119 or 121, by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., asparagine, glutamine, serine, threonine, tyrosine, cysteine), nonpolar side chains (e.g., glycine, alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein is preferably replaced with another amino acid residue from the same side chain family. Alternatively, in another embodiment, mutations can be introduced randomly along all or part of a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 coding sequence, such as by saturation mutagenesis, and the resultant mutants can be screened for 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252,9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 biological activity to identify mutants that retain activity. Following mutagenesis of SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119 or 121, the encoded protein can be expressed recombinantly and the activity of the protein can be determined using the assay described herein.

Another aspect of the invention pertains to the use of isolated nucleic acid molecules which are antisense to the nucleotide sequence of SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119 or 121. An “antisense” nucleic acid comprises a nucleotide sequence which is complementary to a “sense” nucleic acid encoding a protein, e.g., complementary to the coding strand of a double-stranded cDNA molecule or complementary to an mRNA sequence. Accordingly, an antisense nucleic acid can hydrogen bond to a sense nucleic acid. The antisense nucleic acid can be complementary to an entire 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 coding strand, or to only a portion thereof. In one embodiment, an antisense nucleic acid molecule is antisense to a “coding region” of the coding strand of a nucleotide sequence encoding a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428. The term “coding region” refers to the region of the nucleotide sequence comprising codons which are translated into amino acid residues. In another embodiment, the antisense nucleic acid molecule is antisense to a “noncoding region” of the coding strand of a nucleotide sequence encoding 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428. The term “noncoding region” refers to 5′ and 3′ sequences which flank the coding region that are not translated into amino acids (also referred to as 5′ and 3′ untranslated regions).

Given the coding strand sequences encoding 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 disclosed herein, antisense nucleic acids of the invention can be designed according to the rules of Watson and Crick base pairing. The antisense nucleic acid molecule can be complementary to the entire coding region of 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 mRNA, but more preferably is an oligonucleotide which is antisense to only a portion of the coding or noncoding region of 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 mRNA. For example, the antisense oligonucleotide can be complementary to the region surrounding the translation start site of 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 mRNA. An antisense oligonucleotide can be, for example, about 5, 10, 15, 20, 25, 30, 35, 40, 45 or 50 nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xantine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest). Antisense nucleic acid molecules used in the methods of the invention are further described above, in section IV.

In yet another embodiment, the 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6118, 21225, 1420, 32236, 2099, 2150, 26583, 2784, 8941, 9811, 27444, 50566 or 66428 nucleic acid molecules used in the methods of the present invention can be modified at the base moiety, sugar moiety or phosphate backbone to improve, e.g., the stability, hybridization, or solubility of the molecule. For example, the deoxyribose phosphate backbone of the nucleic acid molecules can be modified to generate peptide nucleic acids (see Hyrup B. et al. (1996) Bioorganic & Medicinal Chemistry 4 (1): 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup B. et al. (1996) supra; Perry-O'Keefe et al. (1996) Proc. Natl. Acad. Sci. 93:14670-675.

PNAs of 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 nucleic acid molecules can be used in the therapeutic and diagnostic applications described herein. For example, PNAs can be used as antisense or antigene agents for sequence-specific modulation of gene expression by, for example, inducing transcription or translation arrest or inhibiting replication. PNAs of 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 nucleic acid molecules can also be used in the analysis of single base pair mutations in a gene, (e.g., by PNA-directed PCR clamping); as ‘artificial restriction enzymes’ when used in combination with other enzymes, (e.g., S1 nucleases (Hyrup B. et al. (1996) supra)); or as probes or primers for DNA sequencing or hybridization (Hyrup B. et al. (1996) supra; Perry-O'Keefe et al. (1996) supra).

In another embodiment, PNAs of 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 can be modified, (e.g., to enhance their stability or cellular uptake), by attaching lipophilic or other helper groups to PNA, by the formation of PNA-DNA chimeras, or by the use of liposomes or other techniques of drug delivery known in the art. For example, PNA-DNA chimeras of 140, 1470, 1686, 2089, 2427, 3702, 5891, 6428, 7181, 7660, 25641, 69583, 49863, 8897, 1682, 17667, 9235, 3703, 14171, 10359, 1660,1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 nucleic acid molecules can be generated which may combine the advantageous properties of PNA and DNA. Such chimeras allow DNA recognition enzymes, (e.g., RNAse H and DNA polymerases), to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup B. et al. (1996) supra). The synthesis of PNA-DNA chimeras can be performed as described in Hyrup B. et al. (1996) supra and Finn P. J. et al. (1996) Nucleic Acids Res. 24 (17): 3357-63. For example, a DNA chain can be synthesized on a solid support using standard phosphoramidite coupling chemistry and modified nucleoside analogs, e.g., 5′-(4-methoxytrityl)amino-5′-deoxy-thymidine phosphoramidite, can be used as a between the PNA and the 5′ end of DNA (Mag, M. et al. (1989) Nucleic Acid Res. 17: 5973-88). PNA monomers are then coupled in a stepwise manner to produce a chimeric molecule with a 5′ PNA segment and a 3′ DNA segment (Finn P. J. et al. (1996) supra). Alternatively, chimeric molecules can be synthesized with a 5′ DNA segment and a 3′ PNA segment (Peterser, K. H. et al. (1975) Bioorganic Med. Chem. Lett. 5: 1119-11124).

In other embodiments, the oligonucleotide used in the methods of the invention may include other appended groups such as peptides (e.g., for targeting host cell receptors in vivo), or agents facilitating transport across the cell membrane (see, e.g., Letsinger et al. (1989) Proc. Natl. Acad. Sci. USA 86:6553-6556; Lemaitre et al. (1987) Proc. Natl. Acad. Sci. USA 84:648-652; PCT Publication No. WO88/09810) or the blood-brain barrier (see, e.g., PCT Publication No. WO89/10134). In addition, oligonucleotides can be modified with hybridization-triggered cleavage agents (See, e.g., Krol et al. (1988) Bio-Techniques 6:958-976) or intercalating agents. (See, e.g., Zon (1988) Pharm. Res. 5:539-549). To this end, the oligonucleotide may be conjugated to another molecule, (e.g., a peptide, hybridization triggered cross-linking agent, transport agent, or hybridization-triggered cleavage agent).

Isolated 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 Proteins and Anti-140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 Antibodies Used in the Methods of the Invention

The methods of the invention include the use of isolated 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 proteins, and biologically active portions thereof, as well as polypeptide fragments suitable for use as immunogens to raise anti-140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 antibodies. In one embodiment, native 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 proteins can be isolated from cells or tissue sources by an appropriate purification scheme using standard protein purification techniques. In another embodiment, 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 proteins are produced by recombinant DNA techniques. Alternative to recombinant expression, a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein or polypeptide can be synthesized chemically using standard peptide synthesis techniques.

As used herein, a “biologically active portion” of a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein includes a fragment of a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein having a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 activity. Biologically active portions of a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein include peptides comprising amino acid sequences sufficiently identical to or derived from the amino acid sequence of the 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein, e.g., the amino acid sequence shown in SEQ ID NO:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120 or 122, which include fewer amino acids than the full length 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 proteins, and exhibit at least one activity of a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein. Typically, biologically active portions comprise a domain or motif with at least one activity of the 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein (e.g., the N-terminal region of the 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein that is believed to be involved in the regulation of apoptotic activity). A biologically active portion of a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein can be a polypeptide which is, for example, 25, 50, 75, 100, 125, 150, 175, 200, 250, 300 or more amino acids in length. Biologically active portions of a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein can be used as targets for developing agents which modulate a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 activity.

In a preferred embodiment, the 140, 1470, 1686, 2089, 2427, 3702, 5891, 6428, 7181,7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein used in the methods of the invention has an amino acid sequence shown in SEQ ID NO:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120 or 122. In other embodiments, the 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein is substantially identical to SEQ ID NO:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120 or 122, and retains the functional activity of the protein of SEQ ID NO:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120 or 122, yet differs in amino acid sequence due to natural allelic variation or mutagenesis, as described in detail in subsection V above. Accordingly, in another embodiment, the 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein used in the methods of the invention is a protein which comprises an amino acid sequence at least about 50%, 55%, 60%, 65%, 70%, 75%, 80%, 85%, 90%, 95%, 96%, 97%, 98%, 99% or more identical to SEQ ID NO:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120 or 122.

To determine the percent identity of two amino acid sequences or of two nucleic acid sequences, the sequences are aligned for optimal comparison purposes (e.g., gaps can be introduced in one or both of a first and a second amino acid or nucleic acid sequence for optimal alignment and non-identical sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, or 90% of the length of the reference sequence (e.g., when aligning a second sequence to the 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 amino acid sequence of SEQ ID NO:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120 or 122 having 500 amino acid residues, at least 75, preferably at least 150, more preferably at least 225, even more preferably at least 300, and even more preferably at least 400 or more amino acid residues are aligned). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch (J. Mol. Biol. 48:444-453 (1970)) algorithm which has been incorporated into the GAP program in the GCG software package, using either a Blosum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package, using a NWSgapdna.CMP matrix and a gap weight of 40, 50, 60, 70, or 80 and a length weight of 1, 2, 3, 4, 5, or 6. In another embodiment, the percent identity between two amino acid or nucleotide sequences is determined using the algorithm of E. Meyers and W. Miller (Comput. Appl. Biosci. 4:11-17 (1988)) which has been incorporated into the ALIGN program (version 2.0 or 2.0 U), using a PAM120 weight residue table, a gap length penalty of 12 and a gap penalty of 4.

The methods of the invention may also use 140, 1470, 1686, 2089, 2427, 3702,5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 chimeric or fusion proteins. As used herein, a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 “chimeric protein” or “fusion protein” comprises a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 polypeptide operatively linked to a non-140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 polypeptide. An “140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 polypeptide” refers to a polypeptide having an amino acid sequence corresponding to a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 molecule, whereas a “non-140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 polypeptide” refers to a polypeptide having an amino acid sequence corresponding to a protein which is not substantially homologous to the 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein, e.g., a protein which is different from the 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein and which is derived from the same or a different organism. Within a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 fusion protein the 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 polypeptide can correspond to all or a portion of a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein. In a preferred embodiment, a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 fusion protein comprises at least one biologically active portion of a 140, 1470, 1686, 2089, 2427, 3702, 5891, 6428, 7181,7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein. In another preferred embodiment, a 140, 1470, 1686, 2089, 2427, 3702, 5891, 6428, 7181, 7660, 25641, 69583, 49863, 8897, 1682, 17667, 9235,3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 fusion protein comprises at least two biologically active portions of a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893,33230, 1586, 9943, 16334, 68862, 9011, 14031, 6178, 21225, 1420, 32236, 2099, 2150, 26583, 2784, 8941, 9811, 27444, 50566 or 66428 protein. Within the fusion protein, the term “operatively linked” is intended to indicate that the 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 polypeptide and the non-140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 polypeptide are fused in-frame to each other. The non-140, 1470, 1686, 2089, 2427, 3702, 5891, 6428,7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 polypeptide can be fused to the N-terminus or C-terminus of the 140, 1470, 1686, 2089, 2427, 3702, 5891, 6428,7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 polypeptide.

For example, in one embodiment, the fusion protein is a GST-140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 fusion protein in which the 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 sequences are fused to the C-terminus of the GST sequences. Such fusion proteins can facilitate the purification of recombinant 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428.

In another embodiment, this fusion protein is a 140, 1470, 1686, 2089, 2427, 3702,5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein containing a heterologous signal sequence at its N-terminus. In certain host cells (e.g., mammalian host cells), expression and/or secretion of 140, 1470, 1686, 2089, 2427, 3702,5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 can be increased through use of a heterologous signal sequence.

The 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 fusion proteins used in the methods of the invention can be incorporated into pharmaceutical compositions and administered to a subject in vivo. The 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 fusion proteins can be used to affect the bioavailability of a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 substrate. Use of 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 fusion proteins may be useful therapeutically for the treatment of disorders caused by, for example, (i) aberrant modification or mutation of a gene encoding a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein; (ii) mis-regulation of the 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 gene; and (iii) aberrant post-translational modification of a 140, 1470, 1686, 2089, 2427, 3702, 5891, 6428, 7181, 7660, 25641, 69583, 49863, 8897, 1682, 17667, 9235, 3703, 14171, 10359, 1660,1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein.

Moreover, the 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428-fusion proteins used in the methods of the invention can be used as immunogens to produce anti-140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 antibodies in a subject, to purify 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 ligands and in screening assays to identify molecules which inhibit the interaction of 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 with a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 substrate.

Preferably, a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 chimeric or fusion protein used in the methods of the invention is produced by standard recombinant DNA techniques. For example, DNA fragments coding for the different polypeptide sequences are ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, for example, Current Protocols in Molecular Biology, eds. Ausubel et al. John Wiley & Sons: 1992). Moreover, many expression vectors are commercially available that already encode a fusion moiety (e.g., a GST polypeptide). A 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428-encoding nucleic acid can be cloned into such an expression vector such that the fusion moiety is linked in-frame to the 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein. [00635] The present invention also pertains to the use of variants of the 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 proteins which function as either 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 agonists (mimetics) or as 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 antagonists. Variants of the 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 proteins can be generated by mutagenesis, e.g., discrete point mutation or truncation of a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein. An agonist of the 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 proteins can retain substantially the same, or a subset, of the biological activities of the naturally occurring form of a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein. An antagonist of a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584,9525, 14124, 4469, 8990, 2100, 9288, 64698, 10480, 20893,33230, 1586, 9943, 16334, 68862, 9011, 14031, 6178, 21225, 1420, 32236, 2099, 2150, 26583, 2784, 8941, 9811, 27444, 50566 or 66428 protein can inhibit one or more of the activities of the naturally occurring form of the 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein by, for example, competitively modulating a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428-mediated activity of a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein.

In one embodiment, variants of a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein which function as either 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 agonists (mimetics) or as 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 antagonists can be identified by screening combinatorial libraries of mutants, e.g., truncation mutants, of a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein for 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein agonist or antagonist activity. In one embodiment, a variegated library of 140, 1470, 1686, 2089, 2427, 3702, 5891, 6428, 7181, 7660, 25641, 69583, 49863, 8897, 1682, 17667, 9235, 3703, 14171, 10359, 1660,1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 variants is generated by combinatorial mutagenesis at the nucleic acid level and is encoded by a variegated gene library. A variegated library of 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 variants can be produced by, for example, enzymatically ligating a mixture of synthetic oligonucleotides into gene sequences such that a degenerate set of potential 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 sequences is expressible as individual polypeptides, or alternatively, as a set of larger fusion proteins (e.g., for phage display) containing the set of 140, 1470, 1686, 2089, 2427, 3702, 5891, 6428, 7181, 7660, 25641, 69583, 49863, 8897, 1682, 17667, 9235, 3703, 14171, 10359, 1660,1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 sequences therein. There are a variety of methods which can be used to produce libraries of potential 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 sequences. Methods for synthesizing degenerate oligonucleotides are known in the art (see, e.g., Narang, S. A. (1983) Tetrahedron 39:3; Itakura et al. (1984) Annu. Rev. Biochem. 53:323; Itakura et al. (1984) Science 198:1056; Ike et al. (1983) Nucleic Acid Res. 11:477).

In addition, libraries of fragments of a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein coding sequence can be used to generate a variegated population of 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 fragments for screening and subsequent selection of variants of a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein. In one embodiment, a library of coding sequence fragments can be generated by treating a double stranded PCR fragment of a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 coding sequence with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with SI nuclease, and ligating the resulting fragment library into an expression vector. By this method, an expression library can be derived which encodes N-terminal, C-terminal and internal fragments of various sizes of the 140, 1470, 1686, 2089, 2427, 3702, 5891, 6428, 7181,7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 proteins. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique which enhances the frequency of functional mutants in the libraries, can be used in combination with the screening assays to identify 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 variants (Arkin and Yourvan (1992) Proc. Natl. Acad. Sci. USA 89:7811-7815; Delgrave et al. (1993) Protein Engineering 6(3):327-331).

The methods of the present invention further include the use of anti-140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 antibodies. An isolated 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein, or a portion or fragment thereof, can be used as an immunogen to generate antibodies that bind 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 using standard techniques for polyclonal and monoclonal antibody preparation. A full-length 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein can be used or, alternatively, antigenic peptide fragments of 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 can be used as immunogens. The antigenic peptide of 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 comprises at least 8 amino acid residues of the amino acid sequence shown in SEQ ID NO:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120 or 122 and encompasses an epitope of 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 such that an antibody raised against the peptide forms a specific immune complex with the 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein. Preferably, the antigenic peptide comprises at least 10 amino acid residues, more preferably at least 15 amino acid residues, even more preferably at least 20 amino acid residues, and most preferably at least 30 amino acid residues.

Preferred epitopes encompassed by the antigenic peptide are regions of 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 that are located on the surface of the protein, e.g., hydrophilic regions, as well as regions with high antigenicity.

A 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225,1420, 32236, 2099, 2150, 26583, 2784, 8941, 9811, 27444, 50566 or 66428 immunogen is typically used to prepare antibodies by immunizing a suitable subject, (e.g., rabbit, goat, mouse, or other mammal) with the immunogen. An appropriate immunogenic preparation can contain, for example, recombinantly expressed 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein or a chemically synthesized 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 polypeptide. The preparation can further include an adjuvant, such as Freund's complete or incomplete adjuvant, or similar immunostimulatory agent. Immunization of a suitable subject with an immunogenic 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 preparation induces a polyclonal anti-140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 antibody response.

The term “antibody” as used herein refers to immunoglobulin molecules and immunologically active portions of immunoglobulin molecules, i.e., molecules that contain an antigen binding site which specifically binds (immunoreacts with) an antigen, such as a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893,33230, 1586, 9943, 16334, 68862, 9011, 14031, 6178, 21225, 1420, 32236, 2099, 2150, 26583, 2784, 8941, 9811, 27444, 50566 or 66428. Examples of immunologically active portions of immunoglobulin molecules include F(ab) and F(ab′)2 fragments which can be generated by treating the antibody with an enzyme such as pepsin. The invention provides polyclonal and monoclonal antibodies that bind 140, 1470, 1686, 2089, 2427, 3702,5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 molecules. The term “monoclonal antibody” or “monoclonal antibody composition”, as used herein, refers to a population of antibody molecules that contain only one species of an antigen binding site capable of immunoreacting with a particular epitope of 2784, 8941, 9811, 27444, 50566 or 66428. A monoclonal antibody composition thus typically displays a single binding affinity for a particular 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein with which it immunoreacts. [00643] Polyclonal anti-140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 antibodies can be prepared as described above by immunizing a suitable subject with a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 immunogen. The anti-140, 1470, 1686, 2089, 2427, 3702,5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 antibody titer in the immunized subject can be monitored over time by standard techniques, such as with an enzyme linked immunosorbent assay (ELISA) using immobilized 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428. If desired, the antibody molecules directed against 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 can be isolated from the mammal (e.g., from the blood) and further purified by well known techniques, such as protein A chromatography to obtain the IgG fraction. At an appropriate time after immunization, e.g., when the anti-140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 antibody titers are highest, antibody-producing cells can be obtained from the subject and used to prepare monoclonal antibodies by standard techniques, such as the hybridoma technique originally described by Kohler and Milstein (1975) Nature 256:495-497) (see also, Brown et al. (1981) J. Immunol. 127:539-46; Brown et al. (1980) J. Biol. Chem. 255:4980-83; Yeh et al. (1976) Proc. Natl. Acad. Sci. USA 76:2927-31; and Yeh et al. (1982) Int. J. Cancer 29:269-75), the more recent human B cell hybridoma technique (Kozbor et al. (1983) Immunol Today 4:72), the EBV-hybridoma technique (Cole et al. (1985) Monoclonal Antibodies and Cancer Therapy, Alan R. Liss, Inc., pp. 77-96) or trioma techniques. The technology for producing monoclonal antibody hybridomas is well known (see generally Kenneth, R. H. in Monoclonal Antibodies: A New Dimension In Biological Analyses, Plenum Publishing Corp., New York, N.Y. (1980); Lerner, E. A. (1981) Yale J. Biol. Med. 54:387-402; Gefter, M. L. et al. (1977) Somatic Cell Genet. 3:231-36). Briefly, an immortal cell line (typically a myeloma) is fused to lymphocytes (typically splenocytes) from a mammal immunized with a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784,8941, 9811, 27444, 50566 or 66428 immunogen as described above, and the culture supernatants of the resulting hybridoma cells are screened to identify a hybridoma producing a monoclonal antibody that binds 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428.

Any of the many well known protocols used for fusing lymphocytes and immortalized cell lines can be applied for the purpose of generating an anti-1 40, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 monoclonal antibody (see, e.g., G. Galfre et al. (1977) Nature 266:55052; Gefter et al. (1977) supra; Lerner (1981) supra; and Kenneth (1980) supra). Moreover, the ordinarily skilled worker will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, aminopterin and thymidine (“HAT medium”). Any of a number of myeloma cell lines can be used as a fusion partner according to standard techniques, e.g., the P3-NS1/1-Ag4-1, P3-x63-Ag8.653 or Sp2/O-Ag14 myeloma lines. These myeloma lines are available from ATCC. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428, e.g., using a standard ELISA assay.

Alternative to preparing monoclonal antibody-secreting hybridomas, a monoclonal anti-140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 antibody can be identified and isolated by screening a recombinant combinatorial immunoglobulin library (e.g., an antibody phage display library) with 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 to thereby isolate immunoglobulin library members that bind 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening antibody display library can be found in, for example, Ladner et al. U.S. Pat. No. 5,223,409; Kang et al. PCT International Publication No. WO 92/18619; Dower et al. PCT International Publication No. WO 91/17271; Winter et al. PCT International Publication WO 92/20791; Markland et al. PCT International Publication No. WO 92/15679; Breitling et al. PCT International Publication WO 93/01288; McCafferty et al. PCT International Publication No. WO 92/01047; Garrard et al. PCT International Publication No. WO 92/09690; Ladner et al. PCT International Publication No. WO 90/02809; Fuchs et al. (1991) Bio/technology 9:1370-1372; Hay et al. (1992) Hum. Antibod. Hybridomas 3:81-85; Huse et al. (1989) Science 246:1275-1281; Griffiths et al. (1993) EMBO J 12:725-734; Hawkins et al. (1992) J. Mol. Biol. 226:889-896; Clarkson et al. (1991) Nature 352:624-628; Gram et al. (1992) Proc. Natl. Acad. Sci. USA 89:3576-3580; Garrad et al. (1991) Bio/Technology 9:1373-1377; Hoogenboom et al. (1991) Nuc. Acid Res. 19:4133-4137; Barbas et al. (1991) Proc. Natl. Acad. Sci. USA 88:7978-7982; and McCafferty et al. (1990) Nature 348:552-554.

Additionally, recombinant anti-140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 antibodies, such as chimeric and humanized monoclonal antibodies, comprising both human and non-human portions, which can be made using standard recombinant DNA techniques, are within the scope of the methods of the invention. Such chimeric and humanized monoclonal antibodies can be produced by recombinant DNA techniques known in the art, for example using methods described in Robinson et al. International Application No. PCT/US86/02269; Akira, et al. European Patent Application 184,187; Taniguchi, M., European Patent Application 171,496; Morrison et al. European Patent Application 173,494; Neuberger et al. PCT International Publication No. WO 86/01533; Cabilly et al. U.S. Pat. No. 4,816,567; Cabilly et al. European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Canc. Res. 47:999-1005; Wood et al. (1985) Nature 314:446-449; Shaw et al. (1988) J. Natl. Cancer Inst. 80:1553-1559; Morrison, S. L. (1985) Science 229:1202-1207; Oi et al. (1986) BioTechniques 4:214; Winter U.S. Pat. No. 5,225,539; Jones et al. (1986) Nature 321:552-525; Verhoeyan et al. (1988) Science 239:1534; and Beidler et al. (1988) J. Immunol. 141:4053-4060.

An anti-140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 antibody can be used to detect 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein (e.g., in a cellular lysate or cell supernatant) in order to evaluate the abundance and pattern of expression of the 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 protein. Anti-140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

This invention is further illustrated by the following examples which should not be construed as limiting. The contents of all references, patents and published patent applications cited throughout this application, as well as the Figure and the Sequence Listing is incorporated herein by reference.

### EXAMPLES

### Example 1

### Tissue Distribution of Using Taqman™Analysis

This example describes the TaqMan™ procedure. The Taqman™ procedure is a quantitative, reverse transcription PCR-based approach for detecting mRNA. The RT-PCR reaction exploits the 5′ nuclease activity of AmpliTaq Gold™ DNA Polymerase to cleave a TaqMan™ probe during PCR. Briefly, cDNA was generated from the samples of interest, e.g., heart, kidney, liver, skeletal muscle, and various vessels, and used as the starting material for PCR amplification. In addition to the 5′ and 3′ gene-specific primers, a gene-specific oligonucleotide probe (complementary to the region being amplified) was included in the reaction (i.e., the Taqman™ probe). The TaqMan™ probe includes the oligonucleotide with a fluorescent reporter dye covalently linked to the 5′ end of the probe (such as FAM (6-carboxyfluorescein), TET (6-carboxy-4,7,2′,7′-tetrachlorofluorescein), JOE (6-carboxy-4,5-dichloro-2,7-dimethoxyfluorescein), or VIC) and a quencher dye (TAMRA (6-carboxy-N,N,N′,N′-tetramethylrhodamine) at the 3′ end of the probe.

During the PCR reaction, cleavage of the probe separates the reporter dye and the quencher dye, resulting in increased fluorescence of the reporter. Accumulation of PCR products is detected directly by monitoring the increase in fluorescence of the reporter dye. When the probe is intact, the proximity of the reporter dye to the quencher dye results in suppression of the reporter fluorescence. During PCR, if the target of interest is present, the probe specifically anneals between the forward and reverse primer sites. The 5′-3′ nucleolytic activity of the AmpliTaq™ Gold DNA Polymerase cleaves the probe between the reporter and the quencher only if the probe hybridizes to the target. The probe fragments are then displaced from the target, and polymerization of the strand continues. The 3′ end of the probe is blocked to prevent extension of the probe during PCR. This process occurs in every cycle and does not interfere with the exponential accumulation of product. RNA was prepared using the trizol method and treated with DNase to remove contaminating genomic DNA. cDNA was synthesized using standard techniques. Mock cDNA synthesis in the absence of reverse transcriptase resulted in samples with no detectable PCR amplification of the control gene confirms efficient removal of genomic DNA contamination.

Equivalents

Those skilled in the art will recognize, or be able to ascertain using no more than routine experimentation, many equivalents to the specific embodiments of the invention described herein. Such equivalents are intended to be encompassed by the following claims.

What is claimed:

1. A method for identifying a compound capable of treating a tumorigenic disorder or angiogenic disorder, comprising assaying the ability of the compound to modulate 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 nucleic acid expression or 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 polypeptide activity, thereby identifying a compound capable of treating a tumorigenic disorder or an angiogenic disorder.

2. A method for identifying a compound capable of modulating tumorigenesis or angiogenesis comprising:
a) contacting a cell which expresses 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 with a test compound; and
b) assaying the ability of the test compound to modulate the expression of a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 nucleic acid or the activity of a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 polypeptide, thereby identifying a compound capable of modulating a tumorigenesis or angiogenesis.

3. A method for modulating tumorigenesis or angiogenesis in a cell comprising contacting a cell with a 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 modulator, thereby modulating tumorigenesis or angiogenesis in the cell.

4. The method of claim 2, wherein the cell is selected from a group consisting of an endothelial cell, a stromal cell, an epithelial cell, an angiogenic-tissue derived cell, and a fetal derived cell.

5. The method of claim 3, wherein the 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 modulator is a small organic molecule, peptide, antibody or antisense nucleic acid molecule.

6. The method of claim 3, wherein the 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 modulator is capable of modulating 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 polypeptide activity.

7. The method of claim 6, wherein the 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 modulator is a small organic molecule, peptide, antibody or antisense nucleic acid molecule.

8. The method of claim 6, wherein the 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 modulator is capable of modulating 140, 1470, 1686, 2089, 2427, 3702,5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 nucleic acid expression.

9. A method for treating a subject having a tumorigenic disorder or angiogenic disorder characterized by aberrant 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 polypeptide activity or aberrant 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 nucleic acid expression comprising administering to the subject a 140, 1470, 1686, 2089, 2427, 3702, 5891, 6428,7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 modulator, thereby treating said subject having a tumorigenic disorder or angiogenic disorder.

10. The method of claim 9, wherein said tumorigenic or angiogenic disorder is selected from the group consisting of lung tumors, breast tumors, ovary tumors, colon tumors, and hemangioma.

11. The method of claim 9, wherein said 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 modulator is administered in a pharmaceutically acceptable formulation.

12. The method of claim 9, wherein 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 modulator is a small organic molecule, peptide, antibody or antisense nucleic acid molecule.

13. The method of claim 9, wherein the 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 modulator is capable of modulating 140, 1470, 1686, 2089, 2427, 3702, 5891, 6428, 7181, 7660, 25641, 69583, 49863, 8897, 1682, 17667, 9235, 3703, 14171, 10359, 1660, 1450, 18894, 2088, 32427, 2160, 9252, 9389, 1642, 85269, 10297, 1584, 9525, 14124, 4469, 8990, 2100, 9288, 64698, 10480, 20893, 33230, 1586, 9943, 16334, 68862, 9011, 14031, 6178, 21225, 1420, 32236, 2099, 2150, 26583, 2784, 8941, 9811, 27444, 50566 or 66428 polypeptide activity.
